(19) United States
(12) Patent Application Publication
(10) Pub. No.: US 2002/0160446 A1
(43) Pub. Date: Oct. 31, 2002

(54) NOVEL GENES ENCODING PROTEINS HAVING PROGNOSTIC DIAGNOSTIC PREVENTIVE THERAPEUTIC AND OTHER USES

(76) Inventors: Douglas A. Holtzman, Jamaica Plain, MA (US); David P. Gearing, Victoria (AU); Yang Pan, Bellevue, WA (US)

Correspondence Address: INTELLECTUAL PROPERTY GROUP, MILLENNIUM PHARMACEUTICALS INC., 75 SIDNEY STREET, CAMBRIDGE, MA, 02139, US

(21) Appl. No.: 09/811,088

(22) Filed: Mar. 16, 2001

Related U.S. Application Data
(63) Continuation-in-part of application No. 09/712,726, filed on Nov. 14, 2000.

(51) Int. Cl. C12P 21/02; C07H 21/04; C12N 5/06; C07K 14/705; C07K 14/52

(52) U.S. Cl. 435/69.1; 435/69.5; 435/320.1; 435/325; 530/350; 530/351; 536/23.5

(57) ABSTRACT

The invention relates to the discovery and characterization of several genes and the polypeptides they encode: thymotaxin (Tango-45), Tango-63d, Tango-63e, Tango-67, and huchordin (Tango-66). Thymotaxin is a new member of the C-C family of chemokines. Tango-63d and Tango-63e are two novel polypeptides within the tumor necrosis factor (TNF) receptor superfamily. Tango-67 is related to a number of growth factors, particularly members of the connective tissue growth factor family. Huchordin is related to chordin, a known protein that is involved in the induction of twinned axes, can completely rescue axial development in ventralized embryos, is a potent dorsalizing factor, and plays a crucial role in regulating cell-cell interactions in the organizing centers of head, trunk, and tail development. The invention encompasses nucleic acid molecules encoding nucleic acids and polypeptides of the invention, or mutant forms thereof that encode dysfunctional receptor polypeptides, vectors containing these nucleic acid molecules, cells harboring recombinant DNA molecules encoding nucleic acids or polypeptides of the invention, or mutant forms thereof, host fusion proteins that include functional or dysfunctional polypeptides of the invention, transgenic animals that express nucleic acids or polypeptides of the invention, screening methods and therapeutic methods employing the nucleic acid molecules and polypeptides described above, substantially purified nucleic acids and polypeptides of the invention, and therapeutic compositions containing these nucleic acid molecules and polypeptides.

### CROSS REFERENCES TO RELATED APPLICATIONS

This application is a continuation-in-part (and claims the benefit of priority under 35 USC 120) of the following applications:

1. U.S. application Ser. No. 09/712,726 (filed Nov. 14, 2000), which application claims priority from U.S. Ser. No. 08/820,364 (filed Mar. 12, 1997), now abandoned.

2. U.S. application Ser. No. 09/757,421 (filed Jan. 10, 2001), which application claims priority from U.S. Ser. No. 08/843,652 (filed Apr. 16, 1997), now abandoned.

3. U.S. application Ser. No. 08/843,651 (filed Apr. 16, 1997).

4. U.S. application Ser. No. 09/354,809 (filed Jul. 16, 1999), a divisional of U.S. application Ser. No. 08/938,365 (filed Sep. 26, 1997), now issued.

### TECHNICAL FIELD OF THE INVENTION

This invention relates to polypeptides and the genes encoding them.

### BACKGROUND OF THE INVENTION

The molecular bases underlying many human and animal physiological states (e.g., diseased and homeostatic states of various tissues) remain unknown. Nonetheless, it is well understood that these states result from interactions among the proteins and nucleic acids present in the cells of the relevant tissues. In the past, the complexity of biological systems overwhelmed the ability of practitioners to understand the molecular interactions giving rise to normal and abnormal physiological states. More recently, though, the techniques of molecular biology, transgenic and null mutant animal production, computational biology, and pharmacogenomics have enabled practitioners to discern the role and importance of individual genes and proteins in particular physiological states.

Knowledge of the sequences and other properties of genes (particularly including the portions of genes encoding proteins) and the proteins encoded thereby enables the practitioner to design and screen agents which will affect, prospectively or retrospectively, the physiological state of an animal tissue in a favorable way. Such knowledge also enables the practitioner, by detecting the levels of gene expression and protein production, to diagnose the current physiological state of a tissue or animal and to predict such physiological states in the future. This knowledge furthermore enables the practitioner to identify and design molecules that bind with the polynucleotides and proteins, in vitro, in vivo, or both.

The invention relates, in part, to novel chemokines, growth factors, and modulators of cell proliferation and death (apoptosis), which are essential for at least such physiological processes as embryogenesis, homeostasis, modulation (e.g., initiation and suppression) of the immune response (e.g., the inflammatory response), and modulation of cellular proliferation and differentiation.

Six different types of white blood cells (leukocytes) are typically found in the blood: neutrophils, eosinophils, basophils, monocytes, lymphocytes, and plasma cells. Neutrophils and monocytes are primarily responsible for attacking and destroying invading bacteria, viruses, and other harmful agents. Neutrophils circulate within the bloodstream as mature, functional cells. Monocytes, however, circulate as immature cells that have a limited ability to fight infectious agents. It is only when monocytes are stimulated by chemotactic agents to move through the capillary wall into surrounding tissue that they become fully active. Once monocytes enter the tissues, they begin to swell and their cytoplasm fills with many lysosomes and mitochondria. At this point, monocytes are referred to as macrophages, which are extremely effective phagocytes. Each macrophage can engulf as many as 100 bacterial cells, as well as large particles, including whole red blood cells, malarial parasites, and necrotic tissue.

Chemokines (so named for their action as chemotactic cytokines) are proteins that are involved in the activation of leukocytes and thus, are thought to mediate the inflammatory response (Baggiolini et al., Immunology Today 15:127, 1994; Oppenheim et al., Ann. Rev. Immunol. 9:617, 1991).

Chemokines have been divided into three families on the basis of the chromosomal location of the genes that encode them and the motif formed by four conserved cysteine residues in the mature proteins. Chemokines in which one amino acid separates the first two cysteines are within the “C-X-C” family (and are also referred to as a chemokines). These chemokines are thought to be involved in the chemotaxis of neutrophils, to induce changes in cell shape, and to cause transient increases in intracellular calcium, granule exocytosis, and respiratory burst. Members of this family include interleukin-8 (IL-8), neutrophil activating protein-2 (NAP-2) and granulocyte chemotactic protein (GCP). All known C-X-C chemokines have been mapped to human chromosome 4 and mouse chromosome 5.

Chemokines in which the first two cysteine residues are adjacent to one another are members of the “C-C” family (also known as β chemokines) and are chemotactic for monocytes, but not neutrophils. Recently, it has been shown that these proteins are capable of activating basophils and eosinophils. Chemokines belonging to the C-C family include monocyte chemotactic proteins 1, 2, and 3 (MCP-1, MCP-2, and MCP-3; Van Damme et al., J. Exp. Med. 176:59, 1992; Yoshimura et al. J. Exp. Med. 169:1449, 1989), RANTES, and macrophage inflammatory proteins, including α and β (MIP-1α and MIP-1β), MIP-3, MIP-4, and MIP-1γ(WO 95/17092). All known C-C chemokines have been mapped to human chromosome 17 and mouse chromosome 11.

The third chemokine family currently has only one member: the T cell-specific chemoattractant, lymphotactin, which is chemotactic to lymphocytes (Kelner et al., Science 266:1395, 1994). Unlike the chemokines of the C-C and C-X-C families in which two disulfide bonds stabilize the protein, lymphotactin forms only one disulfide bond. Lymphotactin was mapped to human and mouse chromosome 1.

A variety of cell types are involved in the various states of inflammation. For example, acute infiltrates found after bacterial infection are mainly neutrophilic, while mononuclear cells predominate after infection by an intracellular pathogen. Basophils and eosinophils dominate in both immediate-type allergic response and autoimmune diseases. Increased understanding of the regulation of these various cell types by chemokines will facilitate the development of more effective therapies for disorders related to inflammation.

In multicellular organisms, homeostasis is maintained by balancing the rate of cell proliferation against the rate of cell death. This balance is important in pathophysiologic contexts (for example, in the elimination of virally-infected and radiation-damaged cells). Cell proliferation is influenced by numerous growth factors and the expression of proto-oncogenes, which typically encourage progression through the cell cycle. In contrast, numerous events, including the expression of tumor suppressor genes, can lead to an arrest of cellular proliferation.

In differentiated cells, a particular form of cell death called apoptosis (or programmed cell death (PCD)) is carried out when an internal suicide program is activated. This program can be initiated by a variety of external signals as well as signals that are generated within the cell in response to, for example, genetic damage. Thus, apoptosis of a cell or a group of cells is presumably beneficial to the organism as a whole. For many years, the magnitude of apoptotic cell death was not appreciated because the dying cells are quickly eliminated by phagocytes, without an inflammatory response.

The mechanisms that mediate apoptosis have been intensively studied. These mechanisms involve the activation of endogenous proteases, loss of mitochondrial function, and structural changes such as disruption of the cytoskeleton, cell shrinkage, membrane blebbing, and nuclear condensation, which occurs as the cell's DNA is degraded. Initially, large fragments of DNA (of about 50 kb) are produced, and subsequent cleavage between the nucleosomes produces smaller fragments that appear as a “ladder” following electrophoresis through an agarose gel.

The various signals that trigger apoptosis are thought to bring about these events by converging on a common cell death pathway that is regulated by the expression of genes that are highly conserved from worms, such as C. elegans, to humans. In fact, invertebrate model systems have been invaluable tools in identifying and characterizing the genes that control apoptosis. Through the study of invertebrates and more evolved animals, numerous genes that are associated with cell death have been identified, but the way in which their products interact to execute the apoptotic program is poorly understood.

Currently, four cell surface receptors are known to initiate an apoptotic signal: tumor necrosis factor receptor 1 (TNFR-1, also known as p55-R); the Fas receptor (which is also called CD95 or APO-1) (Boldin et al., Cell 85:803, 1996; Muzio et al., Cell 85:817, 1996); Death Receptor 3 (DR-3 (Chinnaiyan et al., Science 274:990-992, 1996)), which is also known as WSL-1 (Kitson et al., Nature 384:372-375, 1996) or APO-3 (Marsters et al., Current Biol. 6:1669-1676, 1996); and Death Receptor 4 (DR-4; Pan et al., Science 276:111-113, 1997), which binds the APO2/TRAIL ligand.

The Fas/APO-1 receptor and TNFR-1 are classified as members of the TNF/nerve growth factor receptor family and both share an intracellular region of homology designated the “death domain” (Boldin et al., supra; Muzio et al., supra). The TNF/nerve growth factor receptor family is extremely large, and contains molecules that differ in their binding specificities; not all of the molecules in this family bind TNF. Furthermore, the regions that are homologous from one family member to another vary. Two family members may have homologous sequence in the ectodomain, but not in the death domain, or vice-versa.

The death domain of the Fas/APO-1 receptor interacts with FADD (Fas-associating protein with death domain, also known as MORT 1) and RIP (receptor interacting protein), forming a complex that, when joined by Caspase-8, constitutes the Fas/APO-1 death-inducing signalling complex (Boldin et al., supra; Muzio et al., supra). The interaction between Fas/APO-1 and FADD is mediated by their respective C-terminal death domains (Chinnaiyan et al., Cell 81:505-512, 1995).

A second complex that is thought to be involved in cell death forms in association with the intracellular portion of TNFR-1, and includes Caspase-8, TRADD (TNFR-1-associated death domain protein), and FADD/MORT1 (Boldin et al., supra; Muzio et al., supra).

Just as not all members of the TNF receptor family bind TNF (see above), not all members contain a death domain. For example, a receptor termed TNFR-2 is a 75 kDa receptor for the TNF ligand that is not believed to contain a death domain. Thus, this receptor may activate an alternative intracellular signalling pathway that may or may not lead to apoptosis (WO 96/34095; Smith et al., Cell 76:959-962, 1994).

The factors that are known to bind TNFR-1 include TNF-α and TNF-β (also known as lymphotoxin-α), which are related members of a broad family of polypeptide mediators, collectively known as cytokines, that includes the interferons, interleukins, and growth factors (Beutler and Cerami, Ann. Rev. Immunol., 7:625-655, 1989). A subset of these polypeptides are classified as TNF-related cytokines and, in addition to TNF-α and TNF-β, include LT-β and ligands for the Fas and 4-1BB receptors.

TNF-α and TNF-β were first recognized for their anti-tumor activities, but are now known as pleiotropic cytokines that play a role in many biological processes. For example, TNF-α is believed to mediate immunostimulation, autoimmune disease, graft rejection, anti-viral responses, septic shock, cerebral malaria, cytotoxicity, protective responses to ionizing radiation, and growth regulation. TNF-β, which is produced by activated lymphocytes, exhibits similar but not identical biological activities. TNF-α elicits tumor necrosis, mediates anti-viral responses, activates polymorphonuclear leukocytes, and induces the expression of MHC class I antigens and adhesion molecules on endothelial cells.

The size and differentiated characteristics of cellular compartments are controlled in part by the availability of extracellular growth factors. These growth factors can influence cellular replication, cell survival, as well as the function of differentiated end cells. The ability to control the expansion of specific cell types in vivo has demonstrated clinical utility, the best examples being the stimulation of red and white blood cell production by erythropoietin and granulocyte colony stimulating factor, respectively. The utility of growth factors for the treatment of other human disorders (e.g., neurodegeneration) is currently being examined, and it is hoped that in certain instances providing exogenous growth stimuli may arrest or reverse the course of degenerative disorders, or provide for more rapid restoration of function in cases of acute tissue damage (e.g. wound healing).

Secreted growth factors also play an important role in early development. Although the details of this process are incompletely understood and vary considerably from species to species, genetic analysis in model organisms has demonstrated an important role for growth factors in the differentiation of the early embryo. One such molecule is the product of the twisted gastrulation gene (TSG), mutations in which lead to defects in embryogenesis. TSG messenger RNA (mRNA) is present in the early embryo and is important for specification of cell fates along the dorsal midline. TSG has been molecularly characterized, and is a cysteine-rich secreted protein that shows homology to connective tissue growth factor (CTGF). CTGF is itself a mitogen for fibroblasts and shares antigenic determinants with platelet derived growth factor (PDGF).

### SUMMARY OF THE INVENTION

The invention relates to the discovery and characterization of thymotaxin (Tango-45), Tango-63d, Tango-67e, Tango-67, and huchordin (Tango-66).

Thymotaxin is a new member of the C-C family of chemokines. The thymotaxin gene encodes a 93 amino acid polypeptide that is 43% homologous to viral MIP-1α. The amino terminal portion of thymotaxin includes a putative signal sequence, indicating that thymotaxin is a secreted protein. Northern blot analysis of thymotaxin mRNA present in heart and skeletal muscle revealed a more abundant 2.4 kb message and a less abundant 3.5 kb message. The 3.5 kb message is much more abundant in tissues within the immune system, including the thymus, spleen, and small intestine.

Tango-63 includes two novel polypeptides with similarity to members of the TNF receptor superfamily. The first, Tango-63d, is a 440 amino acid polypeptide, and the second, Tango-63e, is a 411 amino acid polypeptide that is identical to Tango-63d, with the exception of a deletion of amino acids 183-211.

Tango-67 is a new soluble growth factor. A form of Tango-67 described herein is a 223 amino acid, cysteine rich polypeptide. Northern blot analysis of Tango-67 mRNA reveals that it is present at varying levels in a wide variety of tissues.

Huchordin (human chordin) is a new human gene that encodes polypeptides similar to chordin, a known protein which is involved in the induction of twinned axes, can completely rescue axial development in ventralized embryos, is a potent dorsalizing factor, and plays a crucial role in regulating cell-cell interactions in the organizing centers of head, trunk, and tail development (Sasai et al., Cell 79 (5): 779-790, 1994). The chordin gene encodes a protein of 941 amino acids with a signal sequence and four Cys-rich domains (Sasai et al.).

Northern blot analysis of huchordin mRNA reveals that the huchordin gene is expressed as an approximately 7.5 kb transcript in adult and fetal liver and as an approximately 4.4 kb transcript in adult brain, heart, and pancreas. An additional approximately 2.7 kb transcript is observed in fetal liver. A cDNA corresponding to huchordin has been cloned (SEQ ID NO: 9). Nucleotides 1 to 2601 (SEQ ID NO: 24) of this cDNA encode an 867 amino acid protein (SEQ ID NO: 10) that has homology to Xenopus chordin (Sasai et al., Cell 79 (5): 779-790, 1994).

The invention features an isolated nucleic acid molecule that encodes the secreted form of human thymotaxin, or a polypeptide having sequence that is at least 85% identical to the sequence of SEQ ID NO: 2: the isolated nucleic acid molecule includes a nucleotide sequence encoding the amino acid sequence of SEQ ID NO: 2; the nucleotide sequence of between nucleotide 1 and 282, inclusive, of SEQ ID NO: 1; and the nucleotide sequence of the thymotaxin encoding cDNA contained in the clone having ATCC accession number 98313.

In other embodiments, the isolated nucleic acid molecule encoding thymotaxin hybridizes to a nucleic acid molecule having the sequence of nucleotides 1 to 282, inclusive, of SEQ ID NO: 1 or its complement; and hybridizes to a nucleic acid molecule having the sequence of the thymotaxin encoding cDNA contained in the clone having ATCC accession number 98313. In other embodiments, the hybridization occurs under stringent conditions.

In another embodiment, the invention features a substantially pure polypeptide of the invention (e.g., a thymotaxin polypeptide that is soluble under physiological conditions, a thymotaxin polypeptide which includes a signal sequence, a thymotaxin polypeptide that inhibits proliferation of a myeloid or lymphoid progenitor cell, a thymotaxin polypeptide that includes an amino acid sequence that is at least 85% identical to the amino acid sequence of residues 25 to 94 of SEQ ID NO: 2, a thymotaxin polypeptide that includes an amino acid sequence that is at least 86% identical to the amino acid sequence of residues 30 to 94 of SEQ ID NO: 2, a thymotaxin polypeptide that includes an amino acid sequence that is at least 85% identical to the amino acid sequence of SEQ ID NO: 2, and a thymotaxin polypeptide that includes an amino acid sequence that is identical to the amino acid sequence of SEQ ID NO: 2).

The invention also features an isolated nucleic acid molecule comprising a nucleotide sequence encoding a polypeptide that is at least 85% identical to SEQ ID NO: 4; and an isolated nucleic acid molecule comprising a nucleotide sequence encoding a polypeptide that is at least 85% identical to SEQ ID NO: 6.

In other aspect, the invention features: an isolated nucleic acid molecule that includes the nucleotide sequence of SEQ ID NO: 3, and that encodes the amino acid sequence of SEQ ID NO: 4; an isolated nucleic acid molecule that includes the nucleotide sequence of SEQ ID NO: 5, and that encodes the amino acid sequence of SEQ ID NO: 6; an isolated nucleic acid molecule that includes the molecule deposited with the American Type Culture Collection and assigned accession number 98368; and an isolated nucleic acid molecule that includes the molecule deposited with the American Type Culture Collection and assigned accession number 98367.

The invention features an isolated nucleic acid molecule that hybridizes under stringent conditions to a nucleic acid molecule having the nucleotide sequence of SEQ ID NO: 3, the isolated nucleic acid molecule encoding Tango-63d; an isolated nucleic acid molecule that hybridizes under stringent conditions to a nucleic acid molecule having the nucleotide sequence of SEQ ID NO: 5, the isolated nucleic acid molecule encoding Tango-63e; an isolated nucleic acid molecule that includes a nucleotide sequence that is at least 90% identical to the nucleotide sequence of SEQ ID NO: 3, the isolated nucleic acid molecule encoding Tango-63d; and an isolated nucleic acid molecule that includes a nucleotide sequence which is at least 90% identical to the nucleotide sequence of SEQ ID NO: 5, the isolated nucleic acid molecule encoding Tango-63e.

Also considered within the scope of the invention is a nucleic acid molecule that: hybridizes under stringent conditions to cDNA sequence contained within ATCC Accession No. 98367; hybridizes under stringent conditions to cDNA sequence contained within ATCC Accession No. 98368; is 85% identical to SEQ ID NO: 3 (FIG. 3); is 85% identical to SEQ ID NO: 5 (FIG. 4); is 95% identical to SEQ ID NO: 3; is 95% identical to SEQ ID NO: 5; is 85% identical to cDNA sequence contained within ATCC Accession No. 98367; is 85% identical to cDNA sequence contained within ATCC Accession No. 98368; is 95% identical to cDNA sequence contained within ATCC Accession No. 98367; is 95% identical to DNA sequnce contained within ATCC Accession No. 98368; hybridizes under stringent conditions to nucleotides 128 to 1447 of SEQ ID NO: 3 (FIG. 3); or hybridizes under stringent conditions to nucleotides 128 to 1360 of SEQ ID NO: 5 (FIG. 4). Polypeptides encoded by these nucleic acids are also considered within the scope of the invention.

The invention also features an isolated nucleic acid molecule encoding a Tango-67 polypeptide. For example, the isolated nucleic acid molecule encodes the secreted form of human Tango-67, or a polypeptide having sequence that is at least 85% identical to the sequence of SEQ ID NO: 8. In other embodiments, the isolated nucleic acid molecule includes a nucleotide sequence encoding the amino acid sequence of SEQ ID NO: 8; and includes the nucleotide sequence of between nucleotide 182 and 850, inclusive, of SEQ ID NO: 7.

In other embodiments, the isolated nucleic acid molecule encoding Tango-67 hybridizes to a nucleic acid molecule having the sequence of nucleotides 182 to 850, inclusive, of SEQ ID NO: 7 or its complement. In other embodiments, the hybridization occurs under stringent conditions.

Preferred hybridizing nucleic acid molecules have an activity possessed by Tango-67, e.g., the ability to increase proliferation and/or differentiation of cells.

In another embodiment, the invention features a substantially pure Tango-67 polypeptide (e.g., a Tango-67 polypeptide that is soluble under physiological conditions, a Tango-67 polypeptide which includes a signal sequence, a Tango-67 polypeptide that stimulated proliferation and/or differentiation of a cell, a Tango-67 polypeptide that includes an amino acid sequence that is at least 85% identical to the amino acid sequence of SEQ ID NO: 8, a Tango-67 polypeptide that includes an amino acid sequence that is at least 90% identical to the amino acid sequence of of SEQ ID NO: 8, a Tango-67 polypeptide that includes an amino acid sequence that is at least 95% identical to the amino acid sequence of SEQ ID NO: 8, and a Tango-67 polypeptide that includes an amino acid sequence that is identical to the amino acid sequence of SEQ ID NO: 8.

The invention also features nucleic acid molecules that hybridize, preferably under stringent conditions, to a nucleic acid molecule encoding a huchordin polypeptide (e.g., a nucleic acid molecule having the sequence shown in SEQ ID NO: 9, a nucleic acid molecule (SEQ ID NO: 24) having the sequence of the huchordin encoding portion of the sequence of SEQ ID NO: 9), or a nucleic acid molecule having the sequence of the protein coding portion of ATCC deposit No. 98481. Preferably the hybridizing nucleic acid molecule consists of 400, more preferably 200 nucleotides. Preferred hybridizing nucleic acid molecules have a biological activity possessed by huchordin.

A cDNA corresponding to huchordin has been cloned (SEQ ID NO: 9). Nucleotides 1 to 2601 (SEQ ID NO: 3D) of this cDNA encode an 867 amino acid protein (SEQ ID NO: 10) that has homology to Xenopus chordin (Sasai et al., Cell 79:779, 1994).

The invention also features substantially pure or isolated huchordin polypeptides, including those that correspond to various functional domains of huchordin, or fragments thereof. The polypeptides of the invention encompass amino acid sequences that are substantially identical to the amino acid sequence shown in FIG. 6 (SEQ ID NO: 10).

The invention also features a host cell that includes an isolated nucleic acid molecule encoding a polypeptide of the invention, a nucleic acid vector (e.g., an expression vector, a vector which includes a regulatory element, a vector which includes a regulatory element selected from the group consisting of the cytomegalovirus hCMV immediate early gene, the early promoter of SV40 adenovirus, the late promoter of SV40 adenovirus, the lac system, the trp system, the TAC system, the TRC system, the major operator and promoter regions of phage λ, the control regions of fd coat protein, the promoter for 3-phosphoglycerate kinase, the promoters of acid phosphatase, and the promoters of the yeast α-mating factors, vector which includes a regulatory element which directs tissue-specific expression, a vector which includes a reporter gene, a vector which includes a reporter gene selected from the group selected from the group consisting of β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding β-galactosidase), and xanthine guanine phosphoribosyltransferase (XGPRT), a vector that is a plasmid, a vector that is a virus, a vector that is a retrovirus.

In other embodiments, the invention features a substantially pure polypeptide that includes a first portion and a second portion, the first portion including a polypeptide of the invention and the second portion including a detectable marker.

The invention also features an antibody that selectively binds to a polypeptide of the invention (e.g., a monoclonal antibody).

The invention also features a pharmaceutical composition that includes a polypeptide of the invention.

Also included in the invention are: a method for detecting a polypeptide of the invention in a sample, the method including:

(a) obtaining a biological sample;

(b) contacting the biological sample with an antibody that specifically binds a polypeptide of the invention under conditions that allow the formation of polypeptide-of-the-invention-antibody complexes; and

(c) detecting the complexes, if any, as an indication of the presence of a polypeptide of the invention in the sample.

In another aspect, the invention features a method of identifying a compound that modulates the expression of a nucleic acid or polypeptide of the invention, the method including comparing the level of expression of a nucleic acid or polypeptide of the invention in a cell in the presence and absence of a selected compound, wherein a difference in the level of expression in the presence and absence of the selected compound indicates that the selected compound modulates the expression of a nucleic acid or polypeptide of the invention.

In another aspect, the invention features a method of identifying a compound that modulates the activity of a nucleic acid or polypeptide of the invention, the method including comparing the level of activity of a nucleic acid or polypeptide of the invention in a cell in the presence and absence of a selected compound, wherein a difference in the level of activity in the presence and absence of the selected compound indicates that the selected compound modulates the activity of a nucleic acid or polypeptide of the invention.

The function of a nucleic acid or polypeptide of the invention can be altered either by altering the expression of the nucleic acid or polypeptide of the invention (i.e., altering the amount of nucleic acid or polypeptide of the invention present in a given cell) or by altering the activity of the nucleic acid or polypeptide of the invention.

Polypeptides that exhibit at least 70%, preferably at least 80%, more preferably at least 90%, and most preferably at least 95% of the activity of the polypeptides of the invention described herein are considered within the scope of the invention.

In another aspect, the invention features a method for treating a patient suffering from a disorder associated with excessive expression or activity of a nucleic acid or polypeptide of the invention, the method including administering to the patient a compound that inhibits expression or activity of a nucleic acid or polypeptide of the invention.

The invention also features a method for treating a patient suffering from a disorder associated with insufficient expression or activity of a nucleic acid or polypeptide of the invention, the method including administering to the patient a compound which increases expression or activity of a nucleic acid or polypeptide of the invention.

The invention also features a method for diagnosing a disorder associated with aberrant expression of a nucleic acid or polypeptide of the invention, the method including obtaining a biological sample from a patient and measuring expression of a nucleic acid or polypeptide of the invention in the biological sample, wherein increased or decreased expression of a nucleic acid or polypeptide of the invention in the biological sample compared to a control indicates that the patient suffers from a disorder associated with aberrant expression of a nucleic acid or polypeptide of the invention.

In another aspect the invention features a method for diagnosing a disorder associated with aberrant activity of a nucleic acid or polypeptide of the invention, the method including obtaining a biological sample from a patient and measuring activity of a nucleic acid or polypeptide of the invention in the biological sample, wherein increased or decreased activity of a nucleic acid or polypeptide of the invention in the biological sample compared to a control indicates that the patient suffers from a disorder associated with aberrant activity of a nucleic acid or polypeptide of the invention.

The invention encompasses isolated nucleic acid molecules encoding a polypeptide of the invention or a fragment thereof, vectors containing these nucleic acid molecules, cells harboring recombined DNA encoding a polypeptide of the invention, fusion proteins which include a polypeptide of the invention, transgenic animals which express a nucleic acid or polypeptide of the invention, and recombinant knock-out animals which fail to express a nucleic acid or polypeptide of the invention. Especially preferred are nucleic acid molecules encoding the polypeptides of SEQ ID NO: 2, SEQ ID NO: 4, SEQ ID NO: 6, SEQ ID NO: 8, or SEQ ID NO: 10.

The invention encompasses nucleic acids that have a sequence that is substantially identical to a nucleic acid sequence of the invention. The term “substantially identical” is hereby defined as a polypeptide or nucleic acid having a sequence that has at least 85%, preferably 90%, and more preferably 95%, 98%, 99% or more identity to the sequence of a reference nucleic acid sequence, e.g., the nucleic acid sequence of SEQ ID NO: 1, SEQ ID NO:3, SEQ ID NO: 5, SEQ ID NO: 7, or SEQ ID NO: 9.

The nucleic acid molecules of the invention can be inserted into transcription and/or translation vectors, as described below, which will facilitate expression of the insert. The nucleic acid molecules and the polypeptides they encode can be used directly as diagnostic or therapeutic agents, or (in the case of a polypeptide) can be used to generate antibodies that, in turn, are therapeutically useful. Accordingly, expression vectors containing the nucleic acid molecules of the invention, cells transfected with these vectors, the polypeptides expressed, and antibodies generated, against either the entire polypeptide or an antigenic fragment thereof, are among the preferred embodiments.

When the polypeptides of the invention are administered to a patient, they may be given in a membrane-bound or a soluble, circulating form. Typically, the soluble form of the polypeptide will lack the transmembrane domain. Soluble polypeptides may include any number of leader sequences at the 5′ end; the purpose of these leader sequences being, primarily, to allow expression in a eukaryotic system (see, for example, U.S. Pat. No. 5,082,783).

The invention also encompasses nucleic acid molecules that hybridize, preferably under stringent conditions, to a nucleic acid molecule encoding a polypeptide of the invention (e.g., a nucleic acid molecule having the sequence of SEQ ID NO: 1, SEQ ID NO: 3, SEQ ID NO: 5, SEQ ID NO: 7, or SEQ ID NO: 9). In addition, the invention encompasses nucleic acid molecules that hybridize, preferably under stringent conditions, to nucleic acid molecules having the sequences of nucleic acids of the invention encoding cDNA contained in the clones having ATCC Accession Numbers 98313, 98368, 98367, or 98481. Preferably the hybridizing nucleic acid molecule consists of 400, more preferably 200 nucleotides.

Preferred hybridizing nucleic acid molecules have an activity possessed by a nucleic acid or polypeptide of the invention, e.g., the ability to inhibit myeloid or lymphoid cell proliferation.

The invention also features substantially pure or isolated polypeptides of the invention, including those that correspond to various functional domains of polypeptides of the invention, or fragments thereof. The polypeptides of the invention encompass amino acid sequences that are substantially identical to the amino acid sequences of SEQ ID NO: 2, SEQ ID NO: 4, SEQ ID NO: 6, SEQ ID NO: 8, or SEQ ID NO: 10.

The polypeptides of the invention can also be chemically synthesized, or they can be purified from tissues in which they are naturally expressed, according to standard biochemical methods of purification. The polypeptide can be a naturally occurring, synthetic, or a recombinant molecule consisting of a hybrid with one portion, for example, being encoded by all or part of a Tango-63 gene, and a second portion being encoded by all or part of a second gene. For example, the polypeptide can be fused to a hexa-histidine tag to facilitate purification of bacterially expressed protein, or to a hemagglutinin (HA) tag to facilitate purification of protein expressed in eukaryotic cells. The HA tag corresponds to an epitope derived from the influenza hemagglutinin protein (Wilson et al., Cell 37:767, 1984). The polypeptides of the invention can also be fused to another compound (such as polyethylene glycol) that will increase the half-life of the polypeptide within the circulation. Similarly, the receptor polypeptide can be fused to a heterologous polypeptide such as the Fc region of an IgG molecule, or a leader or secretory sequence.

In another aspect, the invention features a chimeric polypeptide that contains a polypeptide encoded by one or more of the nucleic acid molecules described above and a heterologous polypeptide (i.e. a polypeptide that has a sequence other than those described above as polypeptides of the invention).

Also included in the invention are “functional polypeptides”, which possess one or more of the biological functions or activities of polypeptides of the invention. These functions or activities are described in detail below and concern, for example, inhibition of myeloid or lymphoid cell proliferation and/or the ability to bind some or all of the proteins which normally bind to thymotaxin, or induction of apoptosis by, for example, binding some or all of the proteins which normally bind to Tango-63d or Tango-63e. A functional polypeptide is also considered within the scope of the invention if it serves as an antigen for production of antibodies that specifically bind to a polypeptide of the invention. In many cases, functional polypeptides retain one or more domains present in the naturally occurring form of the polypeptide.

The functional polypeptides may contain a primary amino acid sequence that has been modified from those disclosed herein. Preferably these modifications consist of conservative amino acid substitutions, as described herein.

In particular, the invention described herein encompasses polypeptides corresponding to functional domains of polypeptides of the invention (e.g., the death domain), mutated, truncated, or deleted polypeptides that retain at least one of the functional activities of polypeptides of the invention (for example, a polypeptide in which one or more amino acid residues have been substituted, deleted from, or added to the death domain without destroying the ability of the mutant Tango-63d or Tango-63e polypeptides to induce apoptosis, and fusion proteins).

The nucleic acid molecules of the invention can be inserted into a vector, as described below, which will facilitate expression of the insert. The nucleic acid molecules and the polypeptides they encode can be used directly as diagnostic or therapeutic agents, or can be used (directly in the case of the polypeptide or indirectly in the case of a nucleic acid molecule) to generate antibodies that, in turn, are clinically useful as a therapeutic or diagnostic agent. Accordingly, vectors containing the nucleic acid of the invention, cells transfected with these vectors, the polypeptides expressed, and antibodies generated, against either the entire polypeptide or an antigenic fragment thereof, are among the preferred embodiments.

The invention also features antibodies, e.g., monoclonal, polyclonal, and engineered antibodies, which specifically bind polypeptides of the invention. By “specifically binds” is meant an antibody that recognizes and binds to a particular antigen, e.g., a polypeptide of the invention, but which does not substantially recognize or bind to other molecules in a sample, e.g., a biological sample, which includes a polypeptide of the invention.

The invention also features antagonists and agonists of polypeptides of the invention that can inhibit or enhance one or more of the functions or activities of polypeptides of the invention, respectively. Suitable antagonists can include small molecules (i.e., molecules with a molecular weight below about 500), large molecules (i.e., molecules with a molecular weight above about 500), antibodies that bind and “neutralize” polypeptides of the invention (as described below), polypeptides which compete with a native form of a polypeptide of the invention for binding to a protein, e.g., the receptor of a polypeptide of the invention, and nucleic acid molecules that interfere with transcription of nucleic acids of the invention (for example, antisense nucleic acid molecules and ribozymes). Agonists of polypeptides of the invention also include small and large molecules, and antibodies other than “neutralizing” antibodies.

The invention also features molecules that can increase or decrease the expression of a nucleic acid or polypeptide of the invention (e.g., by influencing transcription or translation). Small molecules (i.e., molecules with a molecular weight below about 500), large molecules (i.e., molecules with a molecular weight above about 500), and nucleic acid molecules that can be used to inhibit the expression of a nucleic acid or polypeptide of the invention (for example, antisense and ribozyme molecules) or to enhance their expression (for example, expression constructs that place nucleic acid sequences encoding a nucleic acid or polypeptide of the invention under the control of a strong promoter system), and transgenic animals that express a transgene of the invention.

In addition, the invention features substantially pure polypeptides that functionally interact with polypeptides of the invention, e.g., a receptor of a polypeptide of the invention, and the nucleic acid molecules that encode them.

The polypeptides of the present invention can be employed to identifying putative ligands to which the polypeptides bind. These ligands can be identified, for example, by transfecting a cell population with an appropriate vector from which the polypeptide is expressed, and exposing that cell to various putative ligands. The ligands tested could include, for example, those that are known to interact with members of the TNF receptor superfamily, as well as additional small molecules, cell supernatants, extracts, or other natural products. The polypeptide can also be used to screen an expression library according to standard techniques. This is not to say that the polypeptides of the invention must interact with another molecule in order to exhibit biological activity; the polypeptides may function in a ligand-independent manner.

In the event a ligand is identified, one could then determine whether that ligand acts as a full or partial agonist or antagonist of the polypeptide of the invention using no more than routine pharmacological assays.

The members of a pair of molecules (for example, an antibody-antigen pair or a receptor-ligand pair) are said to “specifically bind” to each other if they bind to each other with greater affinity than to other molecules, even those that are structurally or functionally related to a member of the specific binding pair.

The invention encompasses methods for treating disorders associated with aberrant expression or activity of a nucleic acid or polypeptide of the invention. Thus, the invention includes methods for treating disorders associated with excessive expression or activity of a nucleic acid or polypeptide of the invention. Such methods entail administering a compound that decreases the expression or activity of a nucleic acid or polypeptide of the invention. The invention also includes methods for treating disorders associated with insufficient expression of a nucleic acid or polypeptide of the invention. These methods entail administering a compound that increases the expression or activity of a nucleic acid or polypeptide of the invention.

The invention also features methods for detecting a polypeptide of the invention. Such methods include: obtaining a biological sample; contacting the sample with an antibody that specifically binds a polypeptide of the invention under conditions which permit specific binding; and detecting any antibody-polypeptide-of-the-invention complexes formed.

In addition, the present invention encompasses methods and compositions for the diagnostic evaluation, typing, and prognosis of disorders associated with inappropriate expression or activity of a nucleic acid or polypeptide of the invention. For example, the nucleic acid molecules of the invention can be used as diagnostic hybridization probes to detect, for example, inappropriate expression of a nucleic acid or polypeptide of the invention or mutations in a gene of the invention. Such methods may be used to classify cells by the level of expression of a nucleic acid or polypeptide of the invention.

Alternatively, the nucleic acid molecules can be used as primers for diagnostic PCR analysis for the identification of gene mutations, allelic variations and regulatory defects in a gene of the invention. The present invention further provides for diagnostic kits for the practice of such methods.

The invention features methods of identifying compounds that modulate the expression or activity of a nucleic acid or polypeptide of the invention by assessing the expression or activity of a nucleic acid or polypeptide of the invention in the presence and absence of a selected compound. A difference in the level of expression or activity of a nucleic acid or polypeptide of the invention in the presence and absence of the selected compound indicates that the selected compound is capable of modulating expression or activity of a nucleic acid or polypeptide of the invention. Expression can be assessed either at the level of gene expression (e.g., by measuring mRNA) or protein expression by techniques that are well known to skilled artisans. The activity of a nucleic acid or polypeptide of the invention can be assessed functionally, e.g., by assaying the ability of the compound to inhibit proliferation of myeloid cells.

The preferred methods and materials are described below in examples that are meant to illustrate, not limit, the invention. Skilled artisans will recognize methods and materials that are similar or equivalent to those described herein, and that can be used in the practice or testing of the present invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the detailed description, and from the claims.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a depiction of the full-length and 3′ non-translated nucleotide sequence of thymotaxin and (SEQ ID NO: 1).

FIG. 2 is a depiction of the amino acid sequence (SEQ ID NO: 2) of full-length thymotaxin.

FIG. 3 is a representation of the nucleic acid sequence of Tango-63d (SEQ ID NO: 3; open reading frame from nucleotide 128-1447) and the amino acid sequence of the polypeptide it encodes (SEQ ID NO: 4).

FIG. 4 is a representation of the nucleic acid sequence of Tango-63e (SEQ ID NO: 5; open reading frame from nucleotide 128-1360) and the amino acid sequence of the polypeptide it encodes (SEQ ID NO: 6).

FIG. 5 is a depiction of the nucleotide sequence encoding Tango-67 and 3′ and 5′ non-translated sequence (SEQ ID NO: 7; open reading from nucleotide 182-850) and the amino acid sequence (SEQ ID NO: 8) of Tango-67.

FIG. 6 is a depiction of the sequence of a cDNA encoding huchordin (SEQ ID NO: 9; open reading from nucleotide 1-2601) and the deduced amino sequence (SEQ ID NO: 10) of huchordin.

FIG. 7 is an alignment of a portion of the amino acid sequence of huchordin (upper sequence of each pair) and a portion of amino acid sequence of Xenopus chordin (lower sequence of each pair; SEQ ID NO: 11).

### DETAILED DESCRIPTION OF THE INVENTION

The present invention is based, at least in part, on the discovery of a variety of cDNA molecules which encode proteins which are herein designated thymotaxin, Tango-63d, Tango-63e, Tango-67, and huchordin. These proteins exhibit a variety of physiological activities, and are included in a single application for the sake of convenience. It is understood that the allowability or non-allowability of claims directed to one of these proteins has no bearing on the allowability of claims directed to the others. The characteristics of each of these proteins and the cDNAs encoding them are described separately in the ensuing sections. In addition to the full length mature and immature human proteins described in the following sections, the invention includes fragments, derivatives, and variants of these proteins, as described herein. These proteins, fragments, derivatives, and variants are collectively referred to herein as polypeptides of the invention or proteins of the invention.

As used herein, the term “transfected cell” means any cell into which (or into an ancestor of which) has been introduced, by means of recombinant DNA techniques, a nucleic acid encoding a polypeptide of the invention (e.g., a thymotaxin, Tango-63d, Tango-63e, Tango-67, or huchordin polypeptide).

By “isolated nucleic acid molecule” is meant a nucleic acid molecule that is separated from the 5′ and 3′ coding sequences with which it is immediately contiguous in the naturally occurring genome of an organism. Thus, the term “isolated nucleic acid molecule” includes nucleic acid molecules that are not naturally occurring, e.g., nucleic acid molecules created by recombinant DNA techniques.

The term “nucleic acid molecule” encompasses both RNA and DNA, including cDNA, genomic DNA, and synthetic (e.g., chemically synthesized) DNA. Where single-stranded, the nucleic acid may be a sense strand or an antisense strand.

As used herein, the term “transformed cell” means a cell into which (or into an ancestor of which) has been introduced, by means of recombinant DNA techniques, a nucleic acid molecule encoding a polypeptide of the invention.

The terms “protein” and “polypeptide” are used herein to describe any chain of amino acids, regardless of length or post-translational modification (for example, glycosylation or phosphorylation). Thus, the term “polypeptides of the invention” includes full-length, naturally occurring proteins of the invention (with or without a signal sequence), as well a recombinantly or synthetically produced polypeptide that correspond to a full-length naturally occurring proteins of the invention or to particular domains or portions of a naturally occurring protein. The term also encompasses mature polypeptides of the invention that have an added amino-terminal methionine (useful for expression in prokaryotic cells).

The term “purified” as used herein refers to a nucleic acid or peptide that is substantially free of cellular material, viral material, or culture medium when produced by recombinant DNA techniques, or chemical precursors or other chemicals when chemically synthesized.

Polypeptides or other compounds of interest are said to be “substantially pure” when they are within preparations that are at least 60% by weight (dry weight) the compound of interest. Preferably, the preparation is at least 75%, more preferably at least 90%, and most preferably at least 99%, by weight the compound of interest. Purity can be measured by any appropriate standard method, for example, by column chromatography, polyacrylamide gel electrophoresis, or HPLC analysis.

A polypeptide or nucleic acid molecule is “substantially identical” to a reference polypeptide or nucleic acid molecule if it has a sequence that is at least 85%, preferably at least 90%, and more preferably at least 95%, 98%, or 99% identical to the sequence of the reference polypeptide or nucleic acid molecule.

Where a particular polypeptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide that is 50% identical to the reference polypeptide over its entire length. Of course, many other polypeptides will meet the same criteria.

In the case of polypeptide sequences that are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine.

For polypeptides, the length of the reference polypeptide sequence will generally be at least 16 amino acids, preferably at least 20 amino acids, more preferably at least 25 amino acids, and most preferably 35 amino acids, 50 amino acids, or 100 amino acids. For nucleic acids, the length of the reference nucleic acid sequence will generally be at least 50 nucleotides, preferably at least 60 nucleotides, more preferably at least 75 nucleotides, and most preferably 100 nucleotides or 300 nucleotides.

Sequence identity can be measured using sequence analysis software (for example, the Sequence Analysis Software Package of the Genetics Computer Group, University of Wisconsin Biotechnology Center, 1710 University Avenue, Madison, Wis. 53705), with the default parameters as specified therein.

Thymotaxin

Thymotaxin is a new member of the C-C chemokine family. In general chemokines of the C-C family are chemotactic for monocytes and are capable of activating basophils and eosinophils.

In one embodiment the isolated nucleic acid molecule encoding thymotaxin encodes a thymotaxin polypeptide capable of inhibiting proliferation of progenitor cells, encodes a polypeptide that is chemotactic.

The invention also features a method for inhibiting proliferation of progenitor cells (e.g, actively dividing myeloid cells) in a patient, the method including administering to the patient a substantially pure thymotaxin polypeptide capable of inhibiting progenitor cell proliferation. In other embodiments, the method is carried out in conjunction with surgery, radiation therapy, or chemotherapy.

The invention also features a method for treating inflammation in a patient comprising administering to the patient a compound (e.g., a small molecule, an antisense molecule, an antibody) that inhibits expression or activity of thymotaxin.

The invention also features a method of treating a hyperproliferative myeloid disease (e.g., chronic myelogenous leukemia, polycythemia vera, or a hypermegakaryocytopoietic disorder) in a patient, the method including administering to the patient an effective amount of thymotaxin polypeptide.

The compositions described above can be used to detect and treat inflammation. For example, inflammation can be detected by contacting a biological sample with an antibody that selectively binds thymotaxin; the amount of the antibody selectively bound to the sample provides a measure of the severity of the inflammation. If inflammation is detected (or suspected) one can administer to the patient an antagonist of thymotaxin or an inhibitor of thymotaxin expression, such as those described above, which will inhibit the expression or activity of thymotaxin. Preferably, the antagonist is an antibody or a small molecule.

The invention also features methods for inhibiting cellular proliferation, which can be used to suppress proliferation of actively dividing myeloid cells, e.g., as a treatment for a hyperproliferative myeloid disease. Hyperproliferative myeloid diseases include chronic myelogenous leukemia, polycythemia vera, and a hypermegakaryocytopoietic disorder. In one method, a substantially pure thymotaxin polypeptide is administered, in an amount that is sufficient to inhibit cellular proliferation, to a patient who is suffering from such a disorder. These therapies are discussed further below, and can be used as adjunctive methods, that is, in combination with more traditional therapies including surgery, radiotherapy, or chemotherapy.

Also within the invention are methods for protecting progenitor cells from harm by drugs, radiation, and other therapies which kill rapidly dividing cells. These methods encompass administering a thymotaxin polypeptide to capable of interfering with progenitor cell proliferation.

Thymotaxin as a Chemoprotective Agent

Compounds that bind thymotaxin can be identified using any standard binding assay. For example, candidate compounds can be bound to a solid support. Thymotaxin is then exposed to the immobilized compound and binding is measured (European Patent Application 84/03564).

This is invention also relates to the use of thymotaxin polypeptides to protect myeloid cells, e.g., myeloid progenitor cells, and myeloid stem cells, from drugs or therapies which kill or injure actively dividing cells. Agents that protect myeloid progenitor cells and stem cells in this manner are referred to as chemoprotective agents. Such agents place myeloid progenitor cells (e.g., stem cells) into a protected, slow cell-cycling state, thereby inhibiting or decreasing cell damage or death that could otherwise be caused by cell-cycle active chemotherapy drugs such as cytosine arabinoside, 5-fluorouracil, or hydroxyurea. The use of chemoprotective agents permits the administration of higher doses of chemotherapeutics (or radiation) without compromising the ability of the patient to generate mature functional blood cells.

Many patients who undergo chemotherapy or radiation therapy lose a substantial number of stem cells and other actively dividing myeloid progenitor cells. This loss causes the patients to become susceptible to infection and anemia. One approach for preventing this condition is to inhibit cell proliferation with low doses of a molecule which inhibits cell cycling, thereby protecting the progenitor cells from the effects of chemotherapy and/or radiation therapy. After chemotherapy has ended, the protective treatment is also stopped, which allows the progenitor cells to resume normal proliferation.

Any convenient in vitro or in vivo assay can be used to identify preferred thymotaxin polyp eptides or variants thereof that inhibit progenitor cell proliferation and are thus likely to be a suitable chemoprotective agent. Suitable in vitro assays include those described by Gentile et al. (U.S. Pat. Nos. 5,149,544 and 5,294,544). In these assays, bone marrow or spleen cells are stimulated with, e.g., CSF, in an in vitro system. The inhibitory activity of a candidate molecule (for example, thymotaxin) is assessed by determining the extent to which it decreases CSF-stimulated colony and cluster formation. For example, a thymotaxin polypeptide or variant can be tested as follows. LD cells are plated at a density of 5×10⁵ cells in 0.3% agar culture medium with 10% FBS (Hyclone, Logan, Utah) for assessment of CFU-GM. CFU-GM colonies (>40 cells/group) are stimulated by human rGM-CSF (100U/ml) in combination with human rSLF (50 ng/ml). All colonies are tested in the absence or presence of different concentrations of a thymotaxin polypeptide (or variant thereof) to determine the degree inhibition of proliferation.

Colonies are scored after 14 days incubation at lowered (5%) O₂ tension, and 5% CO₂ in a humidified environment in an ESPEC N₂-O₂-CO₂ incubator BNP-210 (Taoi ESPEC Corp., South Plainfield, N.J.). Three plates are scored per determination.

Suitable molecules are those which are effective to significantly inhibit colony formation by human bone marrow GM progenitor cells at concentrations of at least 200 ng/ml, preferably 100 ng/ml, more preferably 50 ng/ml, or even 10 ng/ml. By assaying a number of thymotaxin polypeptides it is possible to identify a domain of thymotaxin which causes significant inhibition of proliferation.

In addition, inhibition of progenitor cell proliferation can be tested using an in vivo assay. A suitable murine model for assessing progenitor cell proliferation has been described by Cooper et al. (Exp. Hematol. 22:186, 1994). The results of this in vivo model, together with the in vitro assay results, are predictive of the efficacy of the tested molecules in treating patients, e.g., humans.

In suitable in vivo tests, molecules are evaluated for effects on myelopoiesis in mice, with endpoints being nucleated cellularity and differentials in the bone marrow, spleen, and peripheral blood, and absolute numbers and cycling status of myeloid progenitor cells in the marrow and spleen. In each test, groups of C3H/HeJ mice are exposed to a particular test sample. C3H/HeJ mice are preferred because they are relatively insensitive to the effects of endotoxin. Thus, any potential endotoxin contamination in the test samples will not influence the in vivo results.

Thymotaxin polypeptides can be tested as follows, although other assays are also useful. C3H/HeJ mice are obtained from the Jackson Laboratory (Bar Harbor, ME) and housed in a conventional animal facility. The mice are injected intravenously with 0.2 ml/mouse sterile pyrogen-free saline, or the stated amount of a selected thymotaxin polypeptide or variant as described in Mantel et al. (Proc. Natl. Acad. Sci. USA 90:2232, 1993). The mice are sacrificed 24 hours later.

The cycling status of hematopoietic progenitor cells, i.e., the proportion of progenitor cells in DNA synthesis (S phase of the cell cycle), is estimated as described in Maze et al. (J. Immunol. 149:1004, 1992) and Cooper et al. (Exp. Hematol. 22:186, 1994). The high specific activity (20 Ci/mM)-tritiated thymidine (50μCi/mL) (New England Nuclear, Boston, Mass.) kill technique is used, and is based on a calculation in vitro of the reduction in the number of colonies formed after pulse exposure of cells for 20 minutes to “hot” tritiated thymidine as compared with a control such as McCoy's medium or a comparable amount of non-radioactive “cold” thymidine.

Femoral bone marrow is removed from the sacrificed mice, treated with high-specific-activity tritiated thymidine, and plated in 0.3% agar culture medium with 10% FBS in the presence of 10% volume/volume pokeweed mitogen mouse spleen cell cultured medium. Colonies (>40 cells/aggregate) and clusters (3-40 cells) are scored after 7 days of incubation. Three plates are scored for each sample for a statistical analysis. Each mouse is evaluated separately in groups of three mice each.

Preferred thymotaxin polypeptides and variants are effective at a dosage of 200 μg/mouse, 100 μg/mouse, 50 μg/mouse, or even 10 μg/mouse or lower. An effective dosage will reduce progenitor cell cycling by at least 25% or at least 50% or even more.

Chemoprotective thymotaxin polypeptides can be administered to a patient as adjunctive agents before and/or during chemotherapy or radiation therapy to protect progenitor cells from the cytotoxic effects of the chemotherapeutic agents or radiation. Chemoprotective thymotaxin polypeptides place myeloid cells into a protected, slow-cycling state, thereby inhibiting or decreasing cell damage that could otherwise be caused by cell-cycle active chemotherapy drugs such as cytosine arabinoside, 5-fluorouracil, or hydroxyurea. The use of chemoprotective agents permits the administration of higher doses of chemotherapeutics without compromising the ability of the patient to generate mature functional blood cells.

Chemoprotective thymotaxin polypeptides are administered to a patient in the same manner as chemokines generally. Guidance in determining an effective dosage, and formulations for administration can be found hereinbelow.

In chemotherapy, specific protocols may vary, and factors such as tumor size, growth rate, and location of the tumor all affect the course of therapy. Administration of chemotherapeutic agents as well as chemoprotective agents require may required knowledge of the extent of disease, the toxicity of previous treatment courses, and the degree of the expected chemotherapeutic drug toxicity.

Thymotaxin as a Treatment for Inflammation

The thymotaxin polypeptides described herein are likely to mediate inflammation and influence the proliferation of myeloid cells. Accordingly, undesirable inflammation or cellular proliferation can be reduced by the administration of a compound that interferes with thymotaxin expression or function (e.g., an antibody). Compounds that interfere with thymotaxin function may also be used to treat a variety of undesirable inflammatory processes, including atherosclerosis or respiratory infections.

Thymotaxin, like other chemokines (Lord et al., Blood 85:3412, 1995; Laterveer et al., Blood 85:2269, 1995), can be used to mobilize hematopoietic stem cells and progenitor cells from the bone marrow to the peripheral blood. Because stem cells and progenitor cells can be more easily recovered from the peripheral blood than from bone marrow, thymotaxin may be useful for isolating such cells for use in stem cell restorative therapy. Such therapy is useful for patients who have undergone myeloablative and/or myelosuppresive cancer treatments.

Thymotaxin is likely to be involved in the regulation of hematopoietic cells. In particular, thymotaxin, like other chemokines (Graham et al., Nature 344:442, 1994; Broxmeyer et al., J. Immunol. 150:3448, 1993), may be able to inhibit proliferation of hematopoietic stem cells and progenitor cells. Such inhibition can protect the cells from chemotherapeutic damage. Thus, thymotaxin can be used to protect hematopoietic stem cells and progenitor cells from chemotherapeutic damage, e.g, damage during chemotherapy for cancer.

The thymotaxin polypeptides that inhibit progenitor cell proliferation can be used to inhibit hyperproliferative myeloid-based diseases such as chronic myelogenous leukemia, polycythemia vera, and hypermegakaryocytopoietic disorders. Hyperproliferative states in such disorders occur because the progenitor cells are unable to negatively regulate cell growth and replication. Administration of suitable thymotaxin polypeptides is expected to inhibit cell replication resulting in the inhibition of the abnormal cell growth. Dosages of the thymotaxin polypeptides for treating hyperproliferative myeloid-based diseases would be similar to those dosages described above for use of the proteins as adjuncts to chemotherapy.

In addition, thymotaxin polypeptides can be used to prevent myeloid progenitor cells from becoming leukemic as the result of the administration of chemotherapeutic agents. The thymotaxin polypeptides are administered in the same way described above.

Accumulation of neutrophils in tissues is a hallmark of inflammation. Accordingly, undesirable inflammation of the brain associated with disorders such as viral encephalitis, multiple sclerosis, viral or bacterial meningitis, severe head trauma, stroke, neurodegenerative diseases (e.g., Alzheimer's disease and Lou Gehrig's disease), HIV encephalopathy, primary brain tumors (e.g., glioblastomas), Lupus associated cerebritis, and post-seizure brain injury, can be reduced by the administration of a compound that interferes with thymotaxin expression or function. Compounds that interfere with thymotaxin function may also be used to treat other undesirable inflammatory processes, e.g., atherosclerosis or respiratory infections.

In alternate embodiments, anti-inflammation therapy can be designed to reduce the level of endogenous thymotaxin gene expression, e.g., using antisense or ribozyme approaches to inhibit or prevent translation of thymotaxin mRNA transcripts; triple helix approaches to inhibit transcription of the thymotaxin gene; or targeted homologous recombination to inactivate or “knock out” the thymotaxin gene or its endogenous promoter. The antisense, ribozyme, or DNA constructs described herein could be administered directly to the site containing the target cells; e.g., heart, skeletal muscle, thymus, spleen, and small intestine.

Thymotaxin and HIV Infection

Thymotaxin is homologous to the chemokines MIP-1α and MIP-1β. These chemokines have potent suppressive effects on HIV infection due to their effect on virus fusion and entry (Oravecx et al., J. Immunol. 157:1329, 1996. Accordingly, thymotaxin may also be able to block HIV fusion and entry. THus, the invention includes a method for treating HIV infection by administering thymotaxin or a compound capable of binding the thymotaxin receptor to a patient either along or in conjunction with a second HIV therapeutic.

Cloning of the Thymotaxin Gene

A sheared BAC library was constructed from human chromosome 16 (the average fragment size was 3 kb). A number of genomic clones (from a BAC of chromosome 16) were sequenced, and a contig that contained exons with homology to the C-C family of chemokines was identified. The sequences identified were then used to clone and sequence the thymotaxin gene described herein, as follows.

Ninety-six well tissue culture plates were inoculated with individual library transformants in 1 ml of Luria Broth with ampicillin (LB-amp). The resulting cultures were grown for 15 to 16 hours at 37□C. with aeration. A frozen stock was prepared by removing 100 μl of each cell suspension, adding it to 100 μl of 50% glycerol, and mixing. The stocks were stored at −80□ C. DNA was then prepared from the remainder of the culture using the Wizard miniprep system (Promega, Madison Wis.), with modifications for the 96 well plates.

The DNA inserts of a number of clones were sequenced by standard, automated fluorescent dideoxynucleotide sequencing using dye-primer chemistry (applied Biosystems, Inc., Foster City Calif.) on Applied Biosystems 373 and 377 sequenators. The DNA sequences obtained in this manner were screened as follows.

First, each sequence was checked to determine if it was a bacterial, ribosomal, or mitochondrial contaminant. Such sequences were excluded from the subsequent analysis. Second, sequence artifacts, such as vector and repetitive elements, were masked and/or removed from each sequence. Third, the remaining sequences were searched against a copy of the GenBank nucleotide database using the BLASTN program (BLASTN 1.3MP: Altschul et al., J. Mol. Biol. 215:403, 1990). Fourth, the sequences were analyzed against a non-redundant protein database with the BLASTX program (BLASTX 1.3MP: Altschul et al., supra). This protein database is a combination of the Swiss-Prot, PIR, and NCBI GenPept protein databases. The BLASTX program was run using the default BLOSUM-62 substitution matrix with the filter parameter: “xnu+seg”. The score cutoff utilized was 75.

The overlapping clones were assembled into contigs, and the assembled contigs were analyzed using the programs in the GCG package (Genetic Computer Group, University Research Park, 575 Science Drive, Madison, Wis. 53711).

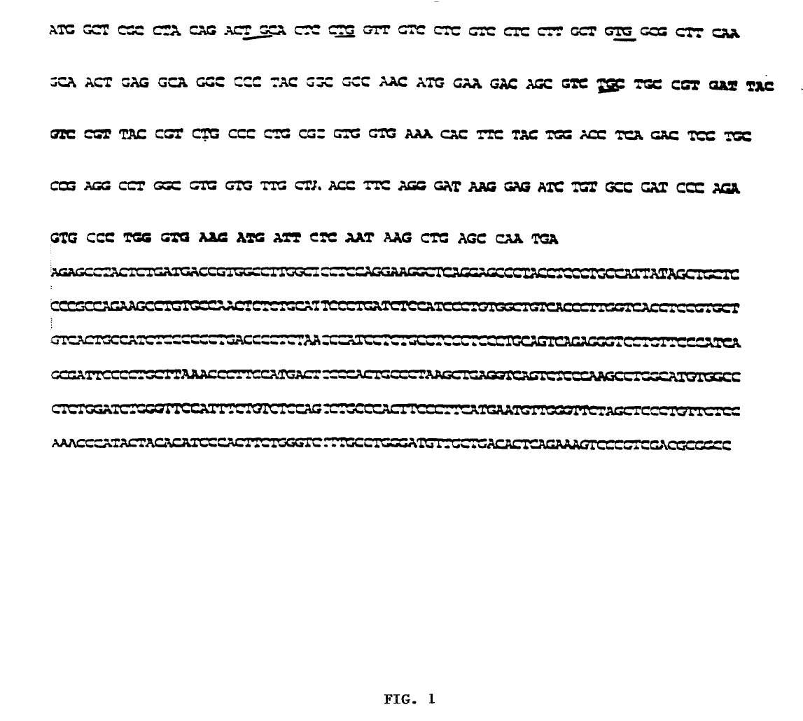

The above-described analysis resulted in the identification of a clone having an open reading frame encoding 93 amino acids (FIG. 1). The protein encoded by this clone was named thymotaxin. The first approximately 24 amino acids in this open reading frame were predicted to be a signal sequence using the method of Von Heijne (J. Membrane Biol. 115:195, 1990). However, by aligning the sequence encoding thymotaxin with the sequence of other β chemokines, the signal sequence was predicted to consist of the first 29 amino acids. The amino-terminal portion of thymotaxin has significant homology (being 43% identical) to viral MIP-1α from Karposi's sarcoma.

Sequences corresponding to the second and third exons were used to generate primers that were then used to screen a cDNA library. A cDNA clone was isolated from a human thymus cDNA library. The cDNA clone, referred to as fthuo45m was deposited with American Type Culture Collection (ATCC), Rockville, Md., and assigned Accession Number 98313.

Based on a published article (Kwitek-Black et al., Nature Genetics, 5:392, 1993) and the integrated genetic map of Chromosome 16 (Genome Directory, Nature, 377:335, 1995), it can be determined that the region to which thymotaxin maps on chromosome 16 overlaps the loci for genes important in the etiology of particular disease conditions, such as Bardet-Biedl Syndrome (BBS), a heterogeneous autosomal recessive disorder characterized by obesity, mental retardation, polydactyly, retinitis pigmentosa, and hypogonadism.

Analysis of thymotaxin expression

Northern blot analysis was used to examine the expression pattern of thymotaxin in human tissues. Multiple tissue Northern blots containing 20 μg of total RNA were purchased from Clontech (Palo Alto Calif.) and hybridized according to the manufacturer's directions to a 0.16 kb fragment of human thymotaxin. For further guidance in performing Northern blot analysis, skilled artisans can consult Chirgwin et al. (Biochemistry 18:5294, 1979).

A transcript of 2.4 kb generated a positive signal upon hybridization and washing under stringent conditions in heart and skeletal muscle tissue. A transcript of 3.5 kb was also seen in these tissues and in brain, placenta, lung, liver, kidney, and pancreas (although it generated a weaker signal). A Northern blot that contained mRNA harvested from tissues within the immune system was also probed under the same conditions described above. The

3.5 kb transcript generated a strong, positive signal in thymus. The message was also expressed in spleen and small intestine, and a weaker signal was evident in prostate, testes, ovary, colon (mucosal lining) tissue and peripheral blood leukocytes.

The two transcripts (i.e. the 2.4 kb and 3.5 kb transcripts) are likely to represent either alternatively spliced forms of thymotaxin or the transcription products of related genes.

Preparation of Soluble thymotaxin

A soluble form of recombinant thymotaxin can be produced in bacteria using the pGEX expression system. The pGEX-thymotaxin can be purified on glutathione agarose and the thymotaxin moiety released by thrombin digestion. Endotoxin can be removed on an Endotoxin BX column (Cape Cod Associates, Falmouth Mass.), and the level of endotoxin remaining can be assessed by the Limulus amebocyte lysate (LAL) assay (also from Cape Cod Associates).

Recombinant, soluble thymotaxin can be produced as follows. First, the coding region of thymotaxin can be amplified with a primer corresponding to a sequence near the 5′ end of the sequence encoding thymotaxin (5′ primer). The 5′ primer, 5′-CGGGATCCGGCCCCTACGGCGCCAACATG-3′ (SEQ ID NO: 12), has an BamHI restriction enzyme cleavage site followed by 24 nucleotides a portion of thymotaxin. The 3′ primer used can be, for example, 5′-CGGAATTCTCATTGGCTCAGCTTATTGAGAATCAT-3′ (SEQ ID NO: 13). This primer has complementary sequences encoding amino acids 85 to 94 preceded by a termination codon and EcoRI site.

These primers pairs can be used for PCR amplification using the following conditions: 94□ C. for 30 seconds; 55□ C. for 30 seconds and 72□ C. for 90 seconds with 30 cycles. The resulting PCR product can be cloned into the GST fusion protein vector pGEX-4T (Pharmacia, Piscataway N.J.). The fusion protein was produced in E. coli and purified according to the protocol supplied by the manufacturer. The thymotaxin construct should produce a protein of approximately 10.5 kD after the cleavage of GST by thrombin.

It is to be understood that while the invention has been described in conjunction with the detailed description thereof, that the foregoing description is intended to illustrate and not limit the scope of the invention, which is defined by the scope of the appended claims. Other aspects, advantages, and modifications are within the scope of the following claims.

Tango-63

The present invention relates to the discovery, identification, and characterization of two nucleic acid molecules that encode novel polypeptides, i.e., Tango-63d and Tango-63e. Tango-63d and Tango-63e are members of the TNF superfamily and may be used in the treatment or amelioration of disorders associated with apoptotic cell death.

Use of Tango-63 Nucleic Acids, Polypeptides, and Antibodies of the Invention in the Diagnosis and Treatment of Disorders Associated with Apoptotic Cell Death

As described herein, the nucleic acids, polypeptides, antibodies, and other reagents of the invention can be used in the diagnosis and treatment of disorders associated with apoptotic cell death. In general, disorders associated with decreased cell death are those in which the expression or activity of Tango-63d and/or Tango-63e can be insufficient. Thus, these disorders can be treated by enhancing the expression or activity of Tango-63d and/or Tango-63e. Conversely, disorders associated with increased cell death are those in which expression or activity of Tango-63d and/or Tango-63e is excessive, and which would respond to treatment regimes in which expression or activity of these genes is inhibited. The disorders amenable to treatment will first be briefly reviewed and a discussion of therapeutic applications will follow (see, for example, “Formulations and Use”).

In addition to the examples provided herein, skilled artisans can consult Thompson (Science 267:1456-1462, 1995) for additional discussion of the disorders associated with apoptotic cell death.

The invention encompasses methods of treatment including a method of treating a patient who has a disorder associated with an abnormal rate of apoptotic cell death by administering a compound that modulates the expression of Tango-63d and/or Tango-63e (at the DNA, mRNA or protein level, e.g., by altering mRNA splicing) or the activity of Tango-63d and/or Tango-63e. Examples of such compounds include small molecules, antisense nucleic acid molecules, ribozymes, and molecules that specifically interact with the polypeptide and thereby act as full or partial agonists or antagonists of its activity.

Disorders that can be treated by altering the expression or activity of the polypeptides of the invention include disorders associated with either an abnormally high or an abnormally low rate or apoptotic cell death (as described further hereinbelow). In addition, T cell mediated diseases, including acquired immune deficiency syndrome (AIDS), autoimmune diseases such as systemic lupus erythrematosis, rheumatoid arthritis, and type I diabetes, septic shock, cerebral malaria, graft rejection, cytotoxicity, cachexia, and inflammation are considered amenable to treatment by altering the expression or activity of a polypeptide of the invention.

A patient who has a disorder associated with an abnormally high rate of apoptotic cell death can be treated by the administration of: a ligand (for example, a naturally occurring or synthetic ligand) that antagonizes Tango-63d or Tango-63e; a compound that decreases the expression of Tango-63d or Tango-63e; a compound that decreases the activity of Tango-63d or Tango-63e; an expression vector that contains a nucleic acid molecule that encodes a nonfunctional Tango-63; or a nonfunctional Tango-63 polypeptide itself. Preferably, the nonfunctional polypeptide will bind any naturally occurring ligand(s) of Tango-63d or Tango-63e or otherwise interfere with the ability of the polypeptides to transduce a signal. Accordingly, the invention features therapeutic compositions that contain the compounds or ligands described above.

Conversely, a patient who has a disorder associated with an abnormally low rate of apoptotic cell death can be treated by the administration of: a ligand (for example, a naturally occurring or synthetic ligand) that activates Tango-63d or Tango-63e (i.e., a ligand that acts as a full or partial agonist of Tango-63d or Tango-63e); a compound that increases the expression of Tango-63d or Tango-63e; a compound that increases the activity of Tango-63d or Tango-63e; an expression vector that contains a nucleic acid molecule encoding Tango-63d or Tango-63e, or by administering either or both of the polypeptides directly to the patient's cells (either in vivo or ex vivo). These methods are described more fully below.

Certain disorders are associated with an increased number of surviving cells, which are produced and continue to survive or proliferate when apoptosis is inhibited. These disorders include cancer, particularly follicular lymphomas, carcinomas associated with mutations in p53, and hormone-dependent tumors such as breast cancer, prostate cancer, and ovarian cancer. As described in the example below, Tango-63 has been mapped to a position that is located in the most frequently lost region of chromosome 8, between markers D8S133 and NEFL. As described in the example below, this region has been implicated in the etiology of numerous cancers, including prostate cancer, colon cancer, non-small cell lung cancer, breast cancer, head and neck cancer, hepatocarcinoma, and bladder cancer.

Additional disorders that are associated with an increased number of surviving cells include autoimmune disorders (such as systemic lupus erythematosus and immune-mediated glomerulonephritis), and viral infections (such as those caused by herpesviruses, poxviruses, and adenoviruses).

Populations of cells are often depleted in the event of viral infection, with perhaps the most dramatic example being the cell depletion caused by the human immunodeficiency virus (HIV). Surprisingly, most T cells that die during HIV infections do not appear to be infected with HIV. Although a number of explanations have been proposed, recent evidence suggests that stimulation of the CD4 receptor results in the enhanced susceptibility of uninfected T cells to undergo apoptosis.

A wide variety of neurological diseases are characterized by the gradual loss of specific sets of neurons. Such disorders are referred to as neurodegenerative diseases and include Alzheimer's disease, Parkinson's disease, amyotrophic lateral sclerosis (ALS), Huntington's disease, retinitis pigmentosa, spinal muscular atrophy, and various forms of cerebellar degeneration. The cell loss in these diseases does not induce an inflammatory response, and apoptosis appears to be the mechanism of cell death.

In addition, a number of hematologic diseases are associated with a decreased production of blood cells. These disorders include anemia associated with chronic disease, aplastic anemia, chronic neutropenia, and the myelodysplastic syndromes. Disorders of blood cell production, such as myelodysplastic syndrome and some forms of aplastic anemia, are associated with increased apoptotic cell death within the bone marrow. These disorders could result from the activation of genes that promote apoptosis, acquired deficiencies in stromal cells or hematopoietic survival factors, or the direct effects of toxins and mediators of immune responses.

Two common disorders associated with cell death are myocardial infarction (which is commonly referred to as a “heart attack”) and cerebral ischemia (which is commonly referred to as “stroke”). In both of these disorders, cells within the central area of ischemia, which is produced in the event of acute loss of blood flow, appear to die rapidly as a result of necrosis. However, outside the central ischemic zone, cells die over a more protracted time period and, morphologically, appear to die by apoptosis.

The present invention encompasses methods and compositions for the diagnostic evaluation, typing, and prognosis of disorders associated with apoptotic cell death and disorders related to abnormal expression or activity or Tango-63d or Tango-63e. The disorder can be associated with either an increase or a decrease in the incidence of apoptotic cell death. For example, the nucleic acid molecules of the invention can be used as diagnostic hybridization probes to detect, for example, expression of Tango-63d or Tango-63e. Such methods can be used to classify cells by their level of Tango-63d or Tango-63e expression. For example, higher Tango-63d or Tango-63e expression may be associated with a higher rate of apoptosis. The present invention further provides for diagnostic kits for the practice of such methods.

In another embodiment, the invention features methods of identifying compounds that modulate apoptotic cell death by modulating the expression or activity of Tango-63d and/or Tango-63e by assessing the expression or activity of Tango-63d and/or Tango-63e in the presence and absence of the compound. A difference in the level of expression or activity of Tango-63d or Tango-63e in the presence of the compound (compared with the level of expression or activity in the absence of the compound) indicates that the compound is capable of modulating the expression or activity of Tango-63d or Tango-63e and thereby useful in, for example, modulating apoptotic cell death. Expression can be assessed either at the level of gene expression (e.g., by measuring mRNA) or protein expression by techniques that are well known to skilled artisans. The activity of Tango-63d or Tango-63e can be assessed functionally, i.e., by assaying the ability of the compound to inhibit apoptosis following activation of the Tango-63d or Tango-63e receptor complexes.

The invention also features a method for determining whether a patient has a disorder associated with an abnormal rate of apoptotic cell death. The method is carried out by quantitating the level of expression of Tango-63d or Tango-63e in a biological sample (e.g., a tumor sample) obtained from the patient. Expression can be assessed by examining the level of mRNA encoding Tango-63d or Tango-63e or the level of Tango-63d or Tango-63e protein. Methods of quantitating mRNA and protein are well known in the art of molecular biology. Methods useful in the present invention include RNAse protection assays, Northern blot analyses, the polymerase chain reaction (PCR, particularly, RT-PCR), and, to assess the level of protein expression, Western blot analyses.

The invention also features a method for determining whether a patient has a disorder associated with a mutation in a gene encoding Tango-63d or Tango-63e. The method is carried out by examining the nucleic acid sequence of Tango-63d or Tango-63e in a sample of DNA obtained from a patient.

The invention also features a method of treating a patient who has a disorder associated with abnormal activity of the Tango-63d or Tango-63e complex. The method is carried out by administering to the patient a compound that modulates the expression or activity of Tango-63d or Tango-63e. The compound can be, for example, a compound that acts as a full or partial agonist of Tango-63d or Tango-63e (which would be administered to increase the activity of Tango-63d or Tango-63e) or as a full or partial antagonist of Tango-63d or Tango-63e (which would be administered to decrease the activity of Tango-63d or Tango-63e). The compound could be a small molecule. To decrease the expression of Tango-63d or Tango-63e, an antisense nucleic acid molecule, or a ribozyme can be administered.

The invention also features therapeutic compositions that include the compounds that are used in the methods of treatment described above. The compounds identified as useful can be naturally occurring or synthetic.

In another aspect, the invention features a method for treating a patient who has a disorder associated with abnormal activity of the Tango-63d or Tango-63e by administering to the patient a compound that mediates oligomerization between Tango-63d or Tango-63e and other molecules that may assemble to form an active complex. These molecules can include TRADD, MORT1, and Caspase-8, or homologues thereof.

The patient who is treated can have any disorder associated with an abnormal level of apoptotic cell death, including acquired immune deficiency syndrome (AIDS), a neurodegenerative disorder, a myelodysplastic syndrome, an ischemic injury, a toxin-induced injury, or a cancer.

The invention also features a method of treating a patient who has a disorder associated with excessive apoptotic cell death by administering to the patient Tango-63d and/or Tango-63e nucleic acid molecules or the Tango-63d and/or Tango-63e polypeptides.

In another aspect, the invention features a method of identifying a compound that modulates expression of Tango-63d and/or Tango-63e by assessing the expression of Tango-63d or Tango-63e in the presence and absence of the compound.

The invention also features a method of treating a patient who has an abnormally low rate of apoptotic cell death. The method is carried out by administering to the patient a compound that mediates oligomerization between Tango-63d and/or Tango-63e and intracellular polypeptides that interact with Tango-63d or Tango-63e to transduce an apoptotic signal that leads to the cell's death.

The invention also features a method of identifying a compound that modulates the activity of Tango-63d and/or Tango-63e by assessing the activity of Tango-63d and/or Tango-63e in the presence and absence of the compound.

In other aspects, the invention includes a method for determining whether a compound modulates oligomerization between Tango-63d and/or Tango-63e and polypeptides that form a complex with these polypeptides by examining oligomerization of Tango-63d and/or Tango-63e and these polypeptides in the presence and absence of the compound. An administered compound may modulate oligomerization between and Tango-63d or Tango-63e and, for example, Caspase-8 or Caspase-8-like polypeptides, TRADD or TRADD-like polypeptides, and FADD/MORT-1 or FADD-MORT-1-like polypeptides.

Whether a Disorder is Mediated by the Expression of Tango-63d or Tango-63e

If one can determine whether a disorder is associated with apoptotic cell death, and whether that cell death is influenced by expression of the polypeptides disclosed herein, it should be possible to determine whether that disorder can be diagnosed or treated with the nucleic acid, polypeptide, or antibody molecules of the invention. A disorder in which there is either insufficient or excessive cell death can be studied by determining whether Tango-63d or Tango-63e are either overexpressed or underexpressed in the affected tissue. The expression levels can be compared from tissue to tissue within a single patient, or between tissue samples obtained from a patient that is ill and one or more patients who are well. If it is determined that either Tango-63d, Tango-63e, or both are either overexpressed or underexpressed, it can be said that the disorder should be amenable to one or more of the treatment methods disclosed herein.

Diagnostic methods in which Tango-63d and Tango-63e are detected in a biological sample can be carried out, for example, by amplifying the nucleic acid molecules within the sample by PCR (the experimental embodiment set forth in Mullis, K. B., 1987, U.S. Pat. No. 4,683,202), followed by the detection of the amplified molecules using techniques well known to those of skill in the art. For example, for detection of the amplified product, the nucleic acid amplification can be performed using radioactively or non-radioactively labeled nucleotides. Alternatively, enough amplified product can be made such that the product can be visualized by standard ethidium bromide staining or by utilizing any other suitable nucleic acid staining method. The resulting amplified sequences can be compared to those which were obtained either from a tissue that is not affected by the disorder, from a person who is well, or that were obtained from the patient before the disorder developed.

The level of expression of Tango-63d and Tango-63e can also be assayed by detecting and measuring transcription. For example, RNA from a cell type or tissue that is known, or suspected to express these polypeptides, can be isolated and tested utilizing the PCR techniques described above.

The analysis of cells taken from culture can be a necessary step in the assessment of cells to be used as part of a cell-based gene therapy technique or, alternatively, to test the effect of compounds on the expression of Tango-63d and Tango-63e. Such analyses can reveal both quantitative and qualitative aspects of the expression pattern of the polypeptides of the invention, including activation or inactivation of their expression.

Where a sufficient quantity of the appropriate cells can be obtained, standard Northern blot or RNAse protection analyses can be performed to determine the level of mRNA encoding polypeptides of the invention, particularly Tango-63d and Tango-63e.

It is also possible to base diagnostic assays and screening assays for therapeutic compounds on detection of Tango-63d polypeptide or Tango-63e polypeptide. Such assays for Tango-63d polypeptide or Tango-63e polypeptide, or peptide fragments thereof will typically involve incubating a sample, such as a biological fluid, a tissue extract, freshly harvested cells, or lysates of cells which have been incubated in cell culture, in the presence of a detectably labeled antibody capable of identifying these gene products (or peptide fragments thereof), and detecting the bound antibody by any of a number of techniques well-known in the art.

The biological sample can be brought in contact with and immobilized onto a solid phase support or carrier such as nitrocellulose, or other solid support capable of immobilizing cells, cell particles, or soluble proteins. The support can then be washed with suitable buffers followed by treatment with the detectably labeled antibody or fusion protein. The solid phase support can then be washed with the buffer a second time to remove unbound antibody or fusion protein. The amount of bound label on solid support can then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material can have virtually any possible structural configuration so long as the coupled molecule is capable of binding to an antigen or antibody. Thus, the support configuration can be spherical, as in a bead, or cylindrical, as in the inside surface of a test tube, or the external surface of a rod. Alternatively, the surface can be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-Tango-63d or anti-Tango-63e antibody or fusion proteins containing these polypeptides can be determined according to well known methods. Those skilled in the art will be able to determine operative and optimal assay conditions for each determination by employing routine experimentation.

With respect to antibodies, one of the ways in which the antibody of the instant invention can be detectably labeled is by linking it to an enzyme for use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.; Voller et al., J. Clin. Pathol. 31:507-520, 1978; Butler, Meth. Enzymol. 73:482-523, 1981; Maggio, E. (ed.), “Enzyme Immunoassay,” CRC Press, Boca Raton, Fla., 1980; Ishikawa, E. et al., (eds.), “Enzyme Immunoassay,” Kgaku Shoin, Tokyo, 1981). The enzyme that is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by calorimetric methods that employ a chromogenic substrate for the enzyme. Detection can also be accomplished by visual comparison of the extent of enzymatic reaction of a substrate in comparison with similarly prepared standards.

Detection can also be accomplished using any of a variety of other immunoassays. For example, by radioactively labeling the antibodies or antibody fragments, it is possible to detect Tango-63d and Tango-63e through the use of a radioimmunoassay (RIA) (see, for example, Weintraub, B., “Principles of Radioimmunoassays, Seventh Training Course on Radioligand Assay Techniques,” The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-pphthaldehyde and fluorescamine.

The antibody can also be detectably labeled using fluorescence emitting metals such as ¹²⁵Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound can be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Still further, the invention encompasses methods and compositions for the treatment of the disorders described above, and any others that are found to be associated with apoptotic cell death. Such methods and compositions are capable of modulating the level of expression of Tango-63d or Tango-63e and/or the level of activity of the gene products.

Numerous ways of altering the expression or activity of the polypeptides of the invention are known to skilled artisans. For example, living cells can be transfected in vivo with the nucleic acid molecules of the invention (or transfected in vitro and subsequently administered to the patient). For example, cells can be transfected with plasmid vectors by standard methods including, but not limited to, liposome- polybrene-, or DEAE dextran-mediated transfection (see, e.g., Felgner et al., Proc. Natl. Acad. Sci. USA 84:7413, 1987; Ono et al., Neurosci. Lett. 117:259, 1990; Brigham et al., Am. J. Med. Sci. 298:278, 1989), electroporation (Neumann et al., EMBO J. 7:841, 1980), calcium phosphate precipitation (Graham et al., Virology 52:456, 1973; Wigler et al., Cell 14:725, 1978; Felgner et al., supra) microinjection (Wolff et al., Science 247:1465, 1990), or velocity driven microprojectiles (“biolistics”).

These methods can be employed to mediate therapeutic application of the molecules of the invention. For example, antisense nucleic acid therapies or ribozyme approaches can be used to inhibit utilization of Tango-63d and/or Tango-63e mRNA; triple helix approaches can also be successful in inhibiting transcription of various polypeptides in the TNF receptor superfamily. Antisense approaches involve the design of oligonucleotides (either DNA or RNA) that are complementary to the mRNA molecules of the invention. The antisense oligonucleotides will bind to the complementary mRNA transcripts and prevent translation. Antisense oligonucleotides must be specific for the mRNA of interest. Accordingly, oligonucleotides disclosed herein as SEQ ID NOs: 8B, 9B, 10B, and 11B are especially preferred. For example, the following oligonucleotides are suitable for specifically binding Tango-63 d or Tango-63 e mRNA: 5′-CATGGCGGTAGGGAACGCTCT-3 ′(SEQ ID NO: 14; the reverse and complement of nucleotides 128-148), 5′-GTTCTGTCCCCGTTGTTCCAT-3′ (SEQ ID NO: 15; the reverse and complement of nucleotides 110-130). The following oligonucleotides are suitable for specifically binding Tango-63d mRNA because they bind to sequences that are not present in Tango-63e:

5′-GGCTTCCCCACTGTGCTTTGT-3′ (SEQ ID NO: 16); and
5′-GGAGGTCACCGTCTCCTCCAC-3′ (SEQ ID NO: 17).

Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA can thus be tested, or triplex formation can be assayed. The ability to hybridize will depend on both the degree of complementarity and the length of the antisense nucleic acid. Generally, the longer the hybridizing nucleic acid, the more base mismatches with an RNA it can contain and still form a stable duplex (or triplex, as the case may be). One skilled in the art can ascertain a tolerable degree of mismatch by use of standard procedures to determine the melting point of the hybridized complex.

Any type of plasmid, cosmid, YAC or viral vector can be used to prepare the recombinant DNA construct that can be introduced directly into the tissue site; for example, the choroid plexus or hypothalamus. Alternatively, viral vectors can be used which selectively infect the desired tissue; (for example, for brain, herpesvirus vectors can be used), in which case administration can be accomplished by another route (for example, systemically).

Methods of designing antisense nucleic acids and introducing them into host cells have been described in, for example, Weinberg et al. (U.S. Pat. No. 4,740,463; hereby incorporated by reference).

Alternatively, the nucleic acid molecules of the invention can be administered so that expression of the Tango-63d and/or Tango-63e occurs in tissues where it does not normally occur, or is enhanced in tissues where it is normally expressed. This application can be used, for example, to suppress apoptotic cell death and thereby treat disorders in which cellular populations are diminished, such as those described herein as “disorders associated with diminished cell survival.” Preferably, the therapeutic nucleic acid (or recombinant nucleic acid construct) is applied to the site where cells are at risk of dying by apoptosis, to the tissue in the larger vicinity, or to the blood vessels supplying these areas.

Ideally, the production of a polypeptide that is a form of Tango-63d or Tango-63e (including forms that are involved in mediating apoptosis) by any gene therapy approach described herein, will result in a cellular level of expression that is at least equivalent to the normal, cellular level of expression of Tango-63d or Tango-63e. Skilled artisans will recognize that these therapies can be used in combination with more traditional therapies, such as surgery, radiotherapy, or chemotherapy. Accordingly, and as described below, the invention features therapeutic compositions that contain the nucleic acid molecules, polypeptides, and antibodies of the invention, as well as compounds that are discovered, as described below, to affect them.

Identification of Compounds that Mediate Oligomerization between Polypeptides within a Tango-63d- or Tango-63e-Containing Complex

It has been shown (see Background of the Invention) that apoptosis can be induced by the formation of specific complexes of polypeptides, for example those that assemble when TNFR-1 or the Fas receptor are bound. Given the conservation between the intracellular domains of TNFR-1, Tango-63d, and Tango-63e, the same or similar polypeptides may assemble with Tango-63d or Tango-63e. Therefore, apoptosis can be inhibited within a cell that contains compounds that specifically inhibit interaction between Tango-63d and/or Tango-63e and polypeptides that would otherwise assemble to form a complex with these polypeptides. Conversely, apoptosis can be stimulated within a cell containing compounds that specifically promote interaction between Tango-63d and/or Tango-63e and one or more additional polypeptides. Accordingly, the invention features a method for treating a patient who has a disorder associated with an abnormally high rate of apoptotic cell death by administering to the patient a compound that inhibits oligomerization between Tango-63d or Tango-63e and other polypeptides. Patients who suffer instead from an abnormally low rate of apoptotic cell death can be treated with a compound that promotes oligomerization between these polypeptides.

The invention also features methods for screening compounds to identify those which increase or decrease the interaction between either Tango-63d and Tango-63e and other polypeptides. One suitable assay for determining whether another polypeptide has become associated with Tango-63d or Tango-63e is an inunuprecipitation assay. A suitable immunoprecipitation assay is described by Kischkel et al. (EMBO J. 14:5579, 1995). Anti-Tango-63d or Anti-Tango-63e antibodies can be used to perform these assays in the presence and absence of selected compounds, and to thereby identify those that increase or decrease association between polypeptides within the Tango-63d and Tango-63e complexes.

Recently, compounds that can penetrate the cell membrane were devised and shown to be capable of controlling the intracellular oligomerization of specific proteins. More specifically, ligands were used to induce intracellular oligomerization of cell surface receptors that lacked their transmembrane and extracellular regions but that contained intracellular signaling domains. Spencer et al. (Science 262:1019-1024, 1993) reported that addition of these ligands to cells in culture resulted in signal transmission and specific target gene activation. Further, these investigators proposed the use of these ligands “wherever precise control of a signal transduction pathway is desired.” For further guidance in the use of synthetic ligands to induce dimerization of proteins, see Belshaw et al. (Proc. Natl. Acad. Sci. USA 93:4604-4607). This approach can be used to induce intracellular oligomerization within a Tango-63d- or Tango-63e-containing complex.

Identification and Characterization of Nucleic Acid Molecules Encoding Tango-63d and Tango-63e

Human prostate epithelial cells were obtained from Clonetics Corporation (San Diego, Calif.) and expanded in culture with Prostate Epithelial Growth Medium (PrEGM; Clonetics) according to the recommendations of the supplier. When the cells reached 80% confluence, they were cultured in Prostate Basal Media (Clonetics) for 24 hours. The prostate cells were then stimulated with PrEGM and cycloheximide (CHI; 40 μg/ml) for 3 hours. Total RNA was isolated using the RNeasy□ Midi Kit (Qiagen; Chatsworth, Calif.), and the polyA⁺ fraction was further purified using Oligotex□ beads (Qiagen).

Three μg of polyA⁺ RNA were used to synthesize a cDNA library using the Superscript□ cDNA synthesis kit (Gibco BRL, Gaithersburg, Md.). Complementary DNA was directionally cloned into the expression plasmid pMET7 using the SalI and NotI sites in the polylinker to construct a plasmid library. Transformants were picked and grown up for single-pass sequencing. Additionally, prostate cDNA was ligated into the SalI/NotI sites of the ZipLox□ vector (Gibco BRL) for construction of a lambda phage cDNA library.

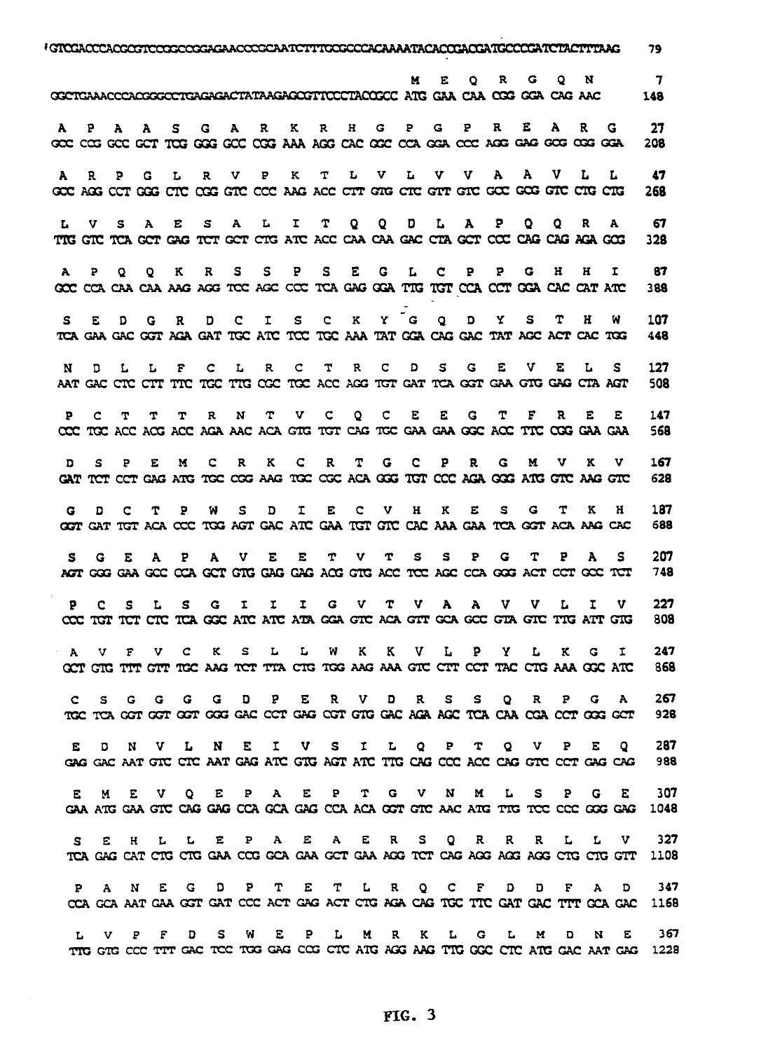

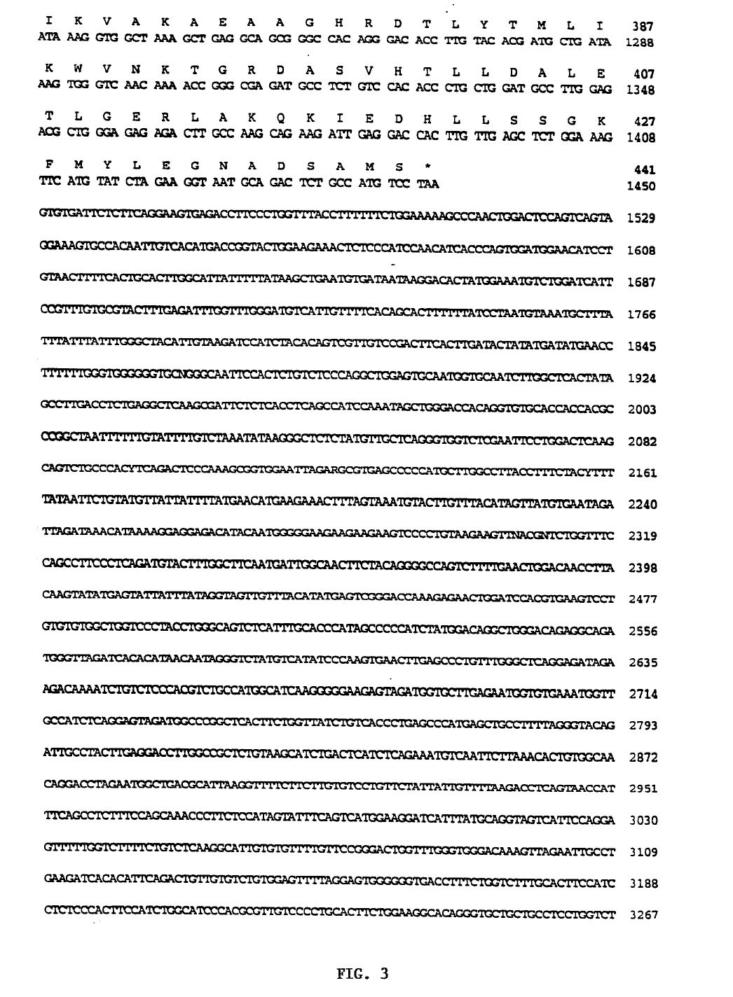

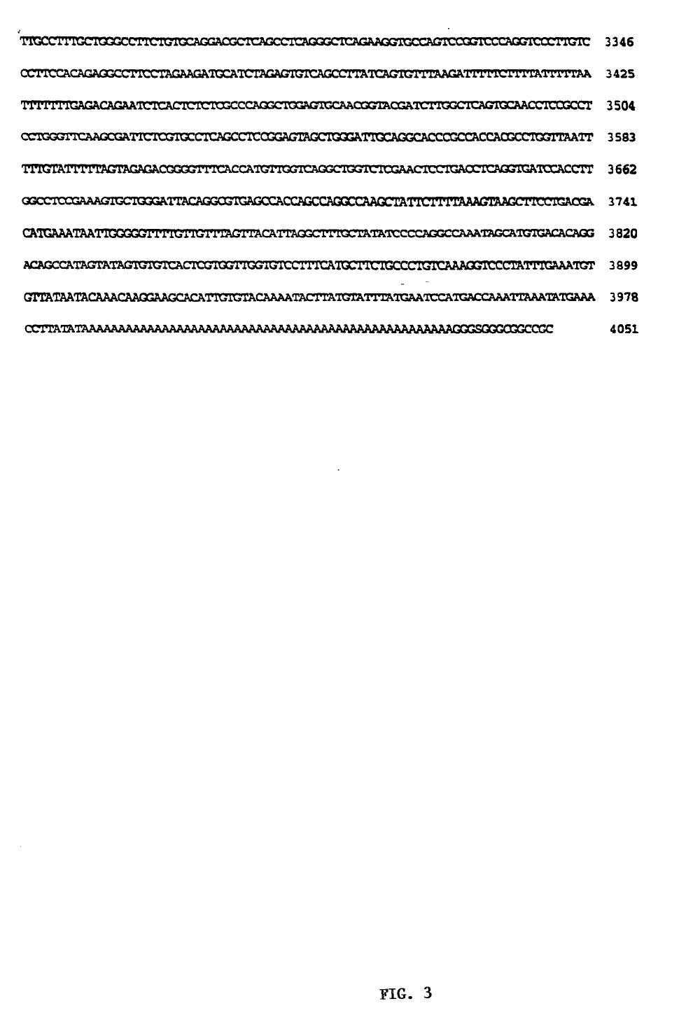

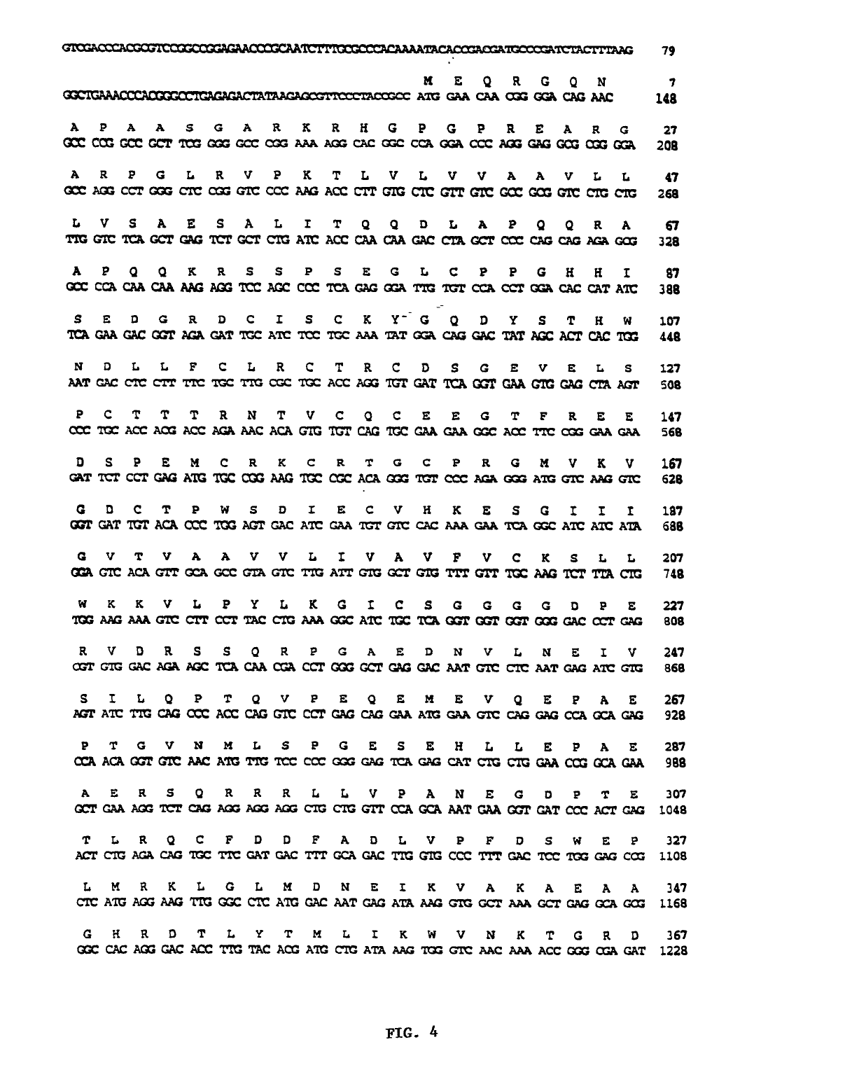

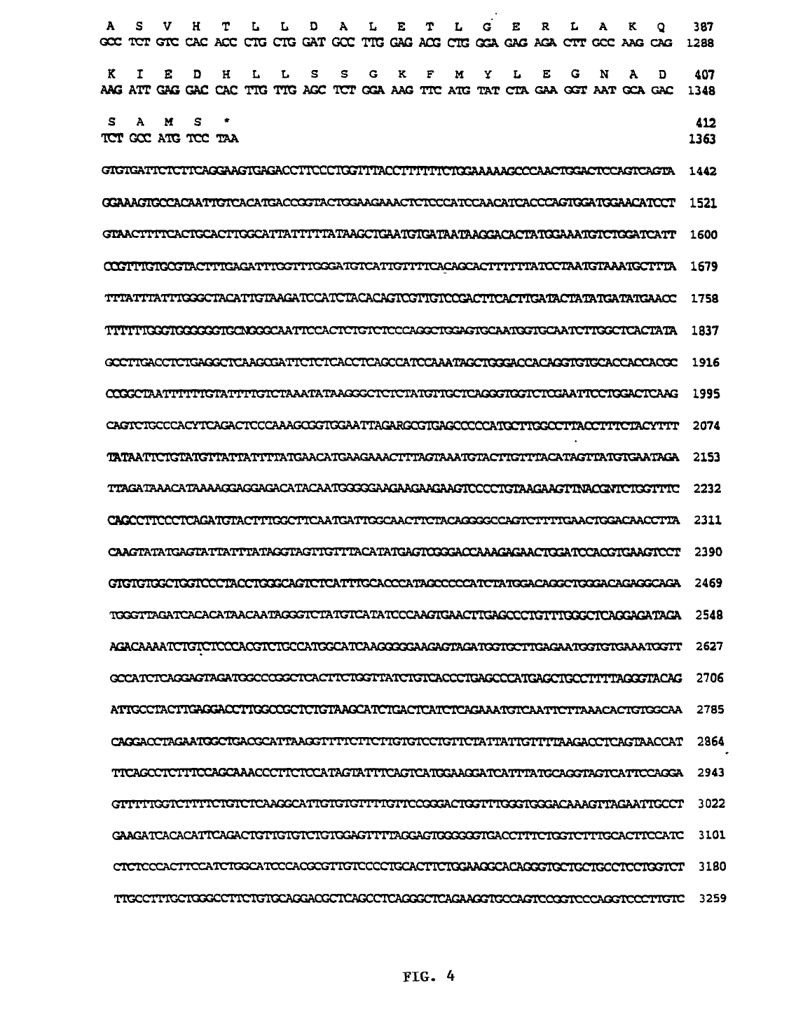

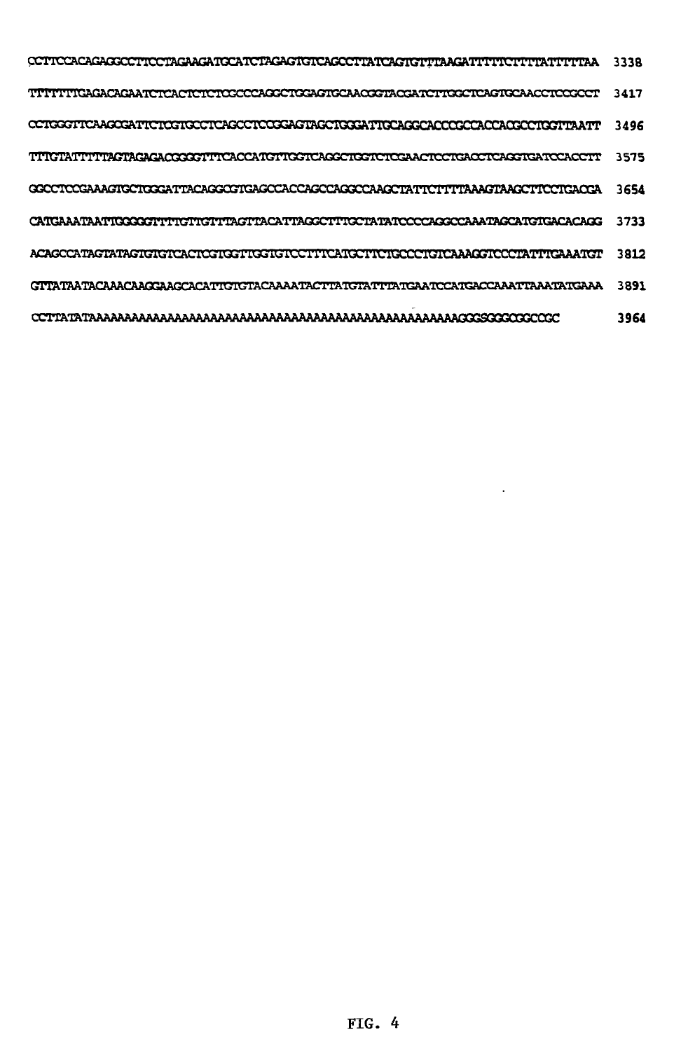

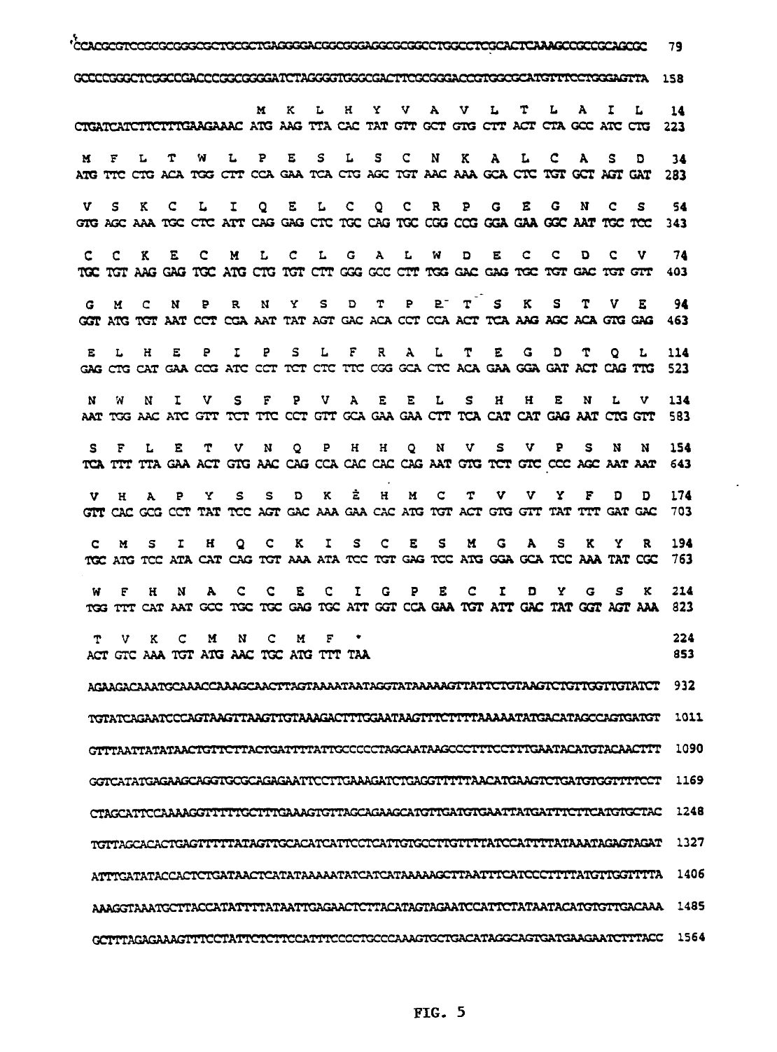

Two different forms of Tango-63 have been identified in the prostate cDNA library through EST sequencing and screening of the lambda phage library for the isolation of additional clones (Tango-63d and Tango-63e). Tango-63d encodes a polypeptide of 440 amino acids (encoded by nucleotides 128 to 1447 of SEQ ID NO: 3 and shown in FIG. 3); and Tango-63e encodes a polypeptide of 411 amino acids (encoded by nucleotides 128 to 1360 of SEQ ID NO: 5 and shown in FIG. 4). The polypeptide encoded by Tango-63e is identical to that encoded by Tango-63d, with the exception of the deletion of amino acids 183-211 (encoded by nucleotides 677-760) in the Tango-63d sequence. The deleted amino acids are those just amino-terminal to the transmembrane domain in Tango-63d. Tango-63d and Tango-63e are novel polypeptides that represent new members of the tumor necrosis factor (TNF) receptor superfamily.

The members of the TNFR receptor superfamily are characterized by the presence of cysteine-rich repeats in their extracellular domains, and the Fas/APO-1 receptor and TNFR-1 also share an intracellular region of homology designated the “death domain” because it is required to signal apoptosis (Itoh and Nagata, J. Biol. Chem. 268:10932-10937, 1993). As described above, this shared death domain suggests that both receptors interact with a related set of signal-transducing molecules.

Tissue Distribution of Tango-63

The expression of Tango-63 (which is subsequently alternatively spliced to produce the novel polypeptides of the invention, Tango-63d and Tango-63e) was analyzed using Northern blot hybridization. A 422 base pair DNA fragment was generated using PCR with the following two oligonucleotides: LRH1 (5′-ATGGAACAACGGGGACAG-3′(SEQ ID NO: 18); nucleotide positions 128-145 in Tango-63d) and LRH3 (5′-TTCTTCGCACTGACACAC-3′(SEQ ID NO: 19); reverse and complement to nucleotide positions 533-550 in Tango-63d for use as a probe. The DNA was radioactively labeled with ³²P-dCTP using the Prime-It□ kit (Stratagene, La Jolla, Calif.) according to the instructions of the supplier. Filters containing human mRNA (MTNI and MTNII from Clontech, Palo Alto, Calif.) were probed in ExpressHyb□ hybridization solution (Clontech) and washed at high stringency. More specifically, the wash was carried out by submerging the filters in 2× SSC, 0.05% SDS at 55□ C. (2×20 minutes) and then in 0.1× SSC, 0.1% SDS at 55□ C. (2×20 minutes).

Tango-63 is expressed as a 4.2 kilobase (kb) transcript in a wide variety of human tissues including heart, placenta, lung, liver, skeletal muscle, kidney, pancreas, spleen, thymus, prostate, testis, ovaries, small intestine, colon, and peripheral blood leukocytes. Expression of Tango-63 was also detectable in the brain, but at significantly lower levels than in other tissues. Additional, but fainter, bands at about 2.2 kb (liver) and 1.0 kb (skeletal muscle) were also observed. These bands could represent additional forms of Tango-63, degradation products, or cross-reacting mRNAs.

An Assay for Tango-63d and Tango-63e Mediated Apoptosis

An assay for Tango-63d- or Tango-63e-mediated apoptosis can be used in screening assays to identify compounds that increase or decrease the degree of apoptosis within a population of cells. The compounds identified using these assays can alter the degree of apoptosis by altering the expression of Tango-63d or Tango-63e, the activity of Tango-63d or Tango-63e, or the way in which these polypeptides interact with other polypeptides. Compounds identified in these assays can be used as therapeutic compounds to treat disorders associated with an abnormal rate of apoptosis.

Assays of apoptosis, particularly when apoptosis is mediated by a polypeptide in the TNF receptor superfamily, generally employ an antibody directed against the polypeptide, which, upon binding, initiates apoptosis. Alternatively, an assay that requires only overexpression of the polypeptide of interest can be performed. An example of such an assay is described below.

The activity of the polypeptides of the invention can be assayed via a cotransfection assay that is based on co-uptake (transfection) with plasmids that encode a polypeptide of the invention. The assay described below is based on the observation that overexpression of TNFR-1, DR-3, and several other death inducing molecules, such as Caspases, is sufficient to cause apoptosis in the absence of other stimuli. The assay described below demonstrates the ability of the novel polypeptides of the invention to diminish the number of cells surviving in culture by activating apoptosis.

β-galactosidase expression assays were performed essentially as described by Kumar et al. (Genes & Dev. 8:1613-1626, 1994). SW480 cells, derived from ahuman colon carcinoma, were cultured in Dulbecco's modified Eagle's medium (DMEM), high glucose, supplemented with 10% fetal calf serum and 100 μg/ml each of penicillin G and streptomycin. The cells were seeded at a density of 3×10⁵ cells/well on 6-well (35 mm) plates and grown in 5% CO₂ at 37□ C. The following day, the cells were transfected with 0.5 μg of pSVβ (Clontech), which carries an insert encoding β-galactosidase, and 2.5 μg of either a control or an experimental plasmid using Lipofectamine□ reagent (Life Technologies) and Opti-MEM□ medium (Life Technologies). The experimental plasmids contained inserts encoding Tango-63d or Tango-63e; the control plasmids were otherwise identical except the Tango-63d or Tango-63e inserts were absent. Thirty-six hours following transfection, the cells were rinsed twice with phosphate-buffered saline (PBS), fixed, and stained for 6 hours or more at 37□ C. If desired, the cells can remain in the staining solution at room temperature for longer periods of time. The staining process consisted of exposure to 1% X-gal, 4 mM potassium ferricyanide, and 2 mM magnesium chloride in PBS. After staining, the cells were examined with a light microscope for the appearance of blue color, indicating successful transfection.

The result of transfection with the control plasmid (encoding β-gal) and either the control or experimental plasmid (encoding Tango-63d or Tango-63e) was assessed by determining the percentage of blue (i.e. transfected) cells in each well or by counting the total number of blue cells in each well. In preliminary experiments, expression of Tango-63d or Tango-63e caused approximately 90% reduction in the number of β-gal positive cells remaining in culture.

Numerous substances are capable of inducing apoptosis in various cell types and can thus be used in assays of apoptosis. These substances include physiological activators, such as TNF family members (for example, Fas ligand, TNFα, and TRAIL/APO2). Cell death can also be induced when growth factors are withdrawn from the medium in which the cells are cultured. Additional inducers of apoptosis include heat shock, viral infection, bacterial toxins, expression of the oncogenes myc, rel, and E1A, expression of tumor suppressor genes, cytolytic T cells, oxidants, free radicals, gamma and ultraviolet irradiation, β-amyloid peptide, ethanol, and chemotherapeutic agents such as Cisplatin, doxorubicin, arabinoside, nitrogen mustard, methotrexate, and vincristine.

Expression of Recombinant Tango-67 in COS Cells

A vector for expression of Tango-67 can be prepared using a vector pcDNAI/Amp (Invitrogen). This vector includes: a SV40 origin of replication, an ampicillin resistance gene, an E. coli replication origin, a CMV promoter followed by polylinker region, a SV40 intron, a and polyadenylation site. A DNA fragment encoding Tango-67 is cloned into the polylinker region of the vector such that Tango-67 expression is under the control of the CMV promoter. A DNA sequence encoding Tango-67 is prepared by PCR amplification of a Tango-67 using primers which include restriction sites that are compatible with the polylinker. The Tango-67 sequence is inserted into the vector. The resulting construct is used to transform E. coli strain SURE (Stratagene, La Jolla, Calif.) and amp resistant colonies are selected. Plasmid DNA is isolated from transformants and examined by restriction analysis the presence of the correct fragment. For expression of the recombinant Tango-67, COS cells are transfected with the expression vector by DEAE-DEXTRAN method and grown is standard tissue culture medium.

Chromosome 8p Loss of Heterozygosity (LOH) and Tango-63

In tumor tissues and cultured cancer cells, loss of heterozygosity (LOH) is much more frequently observed on the short arm of human chromosome 8p than on any other human chromosome. Tumor suppressor genes have been identified in regions of frequent LOH in tumor samples (e.g., p53, Rb, APC, DCC-DPC4). The frequency of LOH reported in the 8p region defined by markers D8S133 to NEFL is greater than 80% in prostate cancer microdissected samples (Vocke et al., Cancer Res. 56:2411-2416, 1996). In addition, loss of 8p is also a frequent event in a number of other cancers including colon cancer, non-small cell lung cancer, breast cancer (Yaremko et al., Genes, Chrom. Cancer 16:189-195, 1996), head and neck cancer (Scholnick et al., J. Natl. Cancer Inst. 88:1676-1682, 1996), hepatocarcinoma (Emi et al., Genes, Chrom. Cancer 7:152-157, 1993), and bladder cancer (Takle et al., Oncogene 12:1083-1087, 1996). Linkage analyses on German breast cancer families' pedigrees have identified a strong linkage in this same region of 8p (Seitz et al., Oncogene 14:741-743, 1997), which has been termed the BRCA3 gene region (Kerangueven et al.).

Tango-63 has been mapped on the Stanford Human Genome Center G3 radiation hybrid panel close to marker D8S 1734 with a LOD score of 6. The mapping was carried out using a pair of primers from the 3′ untranslated region (UTR). The primers are designated t63-f2

(5′-ATGTCATTGTTTTCACAGCA-3′; SEQ ID NO: 20)
(5′-GCTCAAGCGATTCTCTCA-3′; SEQ ID NO: 21).

SEQ ID NO: 20) and t63-r2 This map position is located in the most frequently lost region of chromosome 8 between markers D8S133 and NEFL.

Subsequently, three overlapping yeast artificial chromosomes (YACs) were used to place Tango-63 on the physical map of chromosome 8 between markers WI-6088 and WI-6563.

Tango-67

Tango-67 is a new member of the growth factor superfamily. At the protein sequence level, Tango-67 is related to the product of the Drosophila twisted gastrulation gene and human conective tissue growth factor.

Tango-67 polypeptides are useful for growth promotion. Accordingly they have applications in wound healing, tissue repair, implant fixation, and stimulation of bone growth.

Cloning of the Tango-67 Gene

Human astrocytes (obtained from Clonetics Corporation; San Diego, Calif.) were expanded in culture with Astrocyte Growth Media (AGN; Clonetics) according to the recommendations of the supplier. When the cells reached ˜80-90% confluence, they were stimulated with 200 units/ml Interleukin 1β (Boehringer Mannheim, Indianapolis, Ind.) and cycloheximide (CHI; 40 micrograms/ml) for 4 hours. Total RNA was isolated using the RNeasy Midi Kit (Qiagen; Chatsworth, Calif.), and the poly A⁺ fraction was further purified using Oligotex beads (Qiagen).

Three micrograms of poly A⁺ RNA were used to synthesize a cDNA library using the Superscript cDNA Synthesis kit (Gibco BRL; Gaithersburg, Md.). Complementary DNA was directionally cloned into the expression plasmid pMET7 using the SalI and NotI sites in the polylinker to construct a plasmid library. Transformants were picked and grown up for single-pass sequencing. Additionally, astrocyte cDNA was ligated into the SalI/NotI sites of the ZipLox vector (Gibco BRL) for construction of a lambda phage cDNA library. A partial cDNA clone that encoded a protein with homology to TSG was identified, and additional screening of the phage library led to the isolation of a full-length clone for Tango 67. Tango 67 encodes a protein of 223 amino acids that is 36% identical to D. melanogaster TSG, based on comparisons using the GAP program from GCG (Madison, Wis.).

Analysis of Tango-67 Expression

The expression of Tango 67 was analyzed using Northern blot hybridization. A 410 base pair (bp) DNA fragment was generated using PCR (corresponding to nucleotides 234 to 643 in SEQ ID NO: 7) for use as a probe. The DNA was radioactively labeled with ³²β-dCTP using the Prime-It kit (Strategen, LaJolla, Calif.) according to the instructions of the supplier. Filters containing human mRNA (MTNI and MTNII form Clontech, Palo Alto, Calif.) were probed in ExpressHyb hybridization solution (Clontech) and washed at high stringency according to using a slight variation of the manufacturer's recommendations. The high stringency wash is 2×20 min in 2× SSC, 0.05% SDS at 55□ C.; then 2×20 min in 0.1× SSC, 0.1% SDS at 55□ C.

Tango 67 is expressed at variable levels in all tissues examined (spleen, thymus, prostate, testes, ovary, small intestine, colon, PBLs, heart, brain, placenta, lung, liver, skeletal muscle, kidney and pancreas.) The Tango 67 gene is expressed as two transcripts, an ˜4.4 kilobase (kb) and ˜2.4 kb mRNA, in good agreement with the cDNA clones isolated. The relative levels of the two transcripts vary from tissue to tissue, though with the exception of testes, the 4.4 transcript is significantly more abundant. In the testes the levels of the 4.4 and 2.4 kb mRNAs are approximately the same, and an additional hybridizing transcript is seen at ˜800 bp.

Huchordin

Huchordin, a human protein described here for the first time, is an 867 amino acid protein that is predicted to be a secreted protein. A putative signal sequence encompasses amino acids 1-26 of huchordin.

Huchordin bears homology to Xenopus chordin (Sasai et al., Cell 79:779, 1994). Like Xenopus chordin, huchordin includes several cysteine-rich repeats. Xenopus chordin includes four such repeats (R1, R2, R3, and R4) of 58-74 residues (Sasai et al., Cell 79:779, 1994) each of which includes 10 cysteine residues at conserved positions.

Huchordin contains three intact cysteine-rich repeats (amino acids 51-125; amino acids 696-762; and amino acids 784-844), corresponding to R1, R3, and R4 of chordin. The huchordin cysteine-rich repeat (amino acids 644-674) corresponding to R2 of chordin contains only six of the 10 conserved cys residues and is properly considered a half repeat.

Four potential N-glycosylation sites (217, 351, 365, and 434) are located between RI and R2 in huchordin. Chordin also has four such sites. Two of the potential huchordin N-glycosylation sites N351 at N434 are in positions that are conserved in chordin.

Overall, the huchordin gene described herein has 66% homology at the nucleotide level to the Xenopus chordin gene, and the huchordin protein described herein has 53% homology to Xenopus chordin protein at the amino acid level.

The invention also features molecules that alter the cellular localization of huchordin. Such molecules can be used to treat disorders associated with aberrant cellular localization of huchordin. Huchordin may also be used to inhibit fibrosis or angiogenesis.

In addition, the invention features substantially pure polypeptides that functionally interact with huchordin, e.g., novel members of the TGF-β superfamily, and the nucleic acid molecules that encode them.

Identification Sequencing, and Characterization of a Human Huchordin Gene

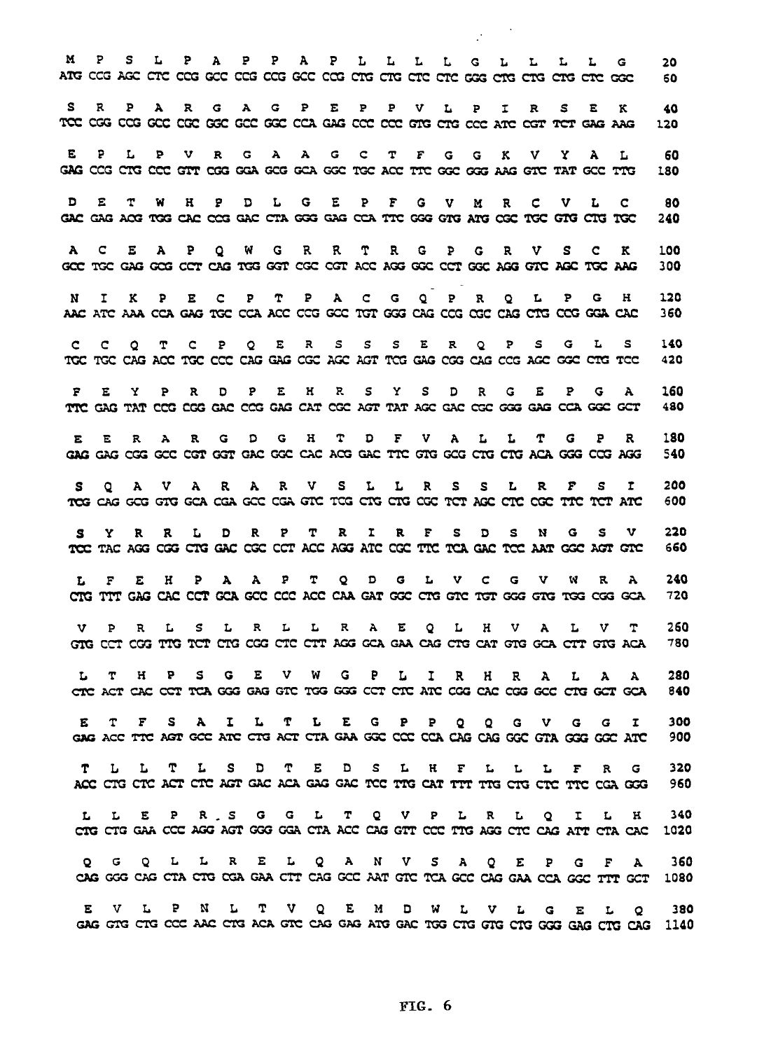

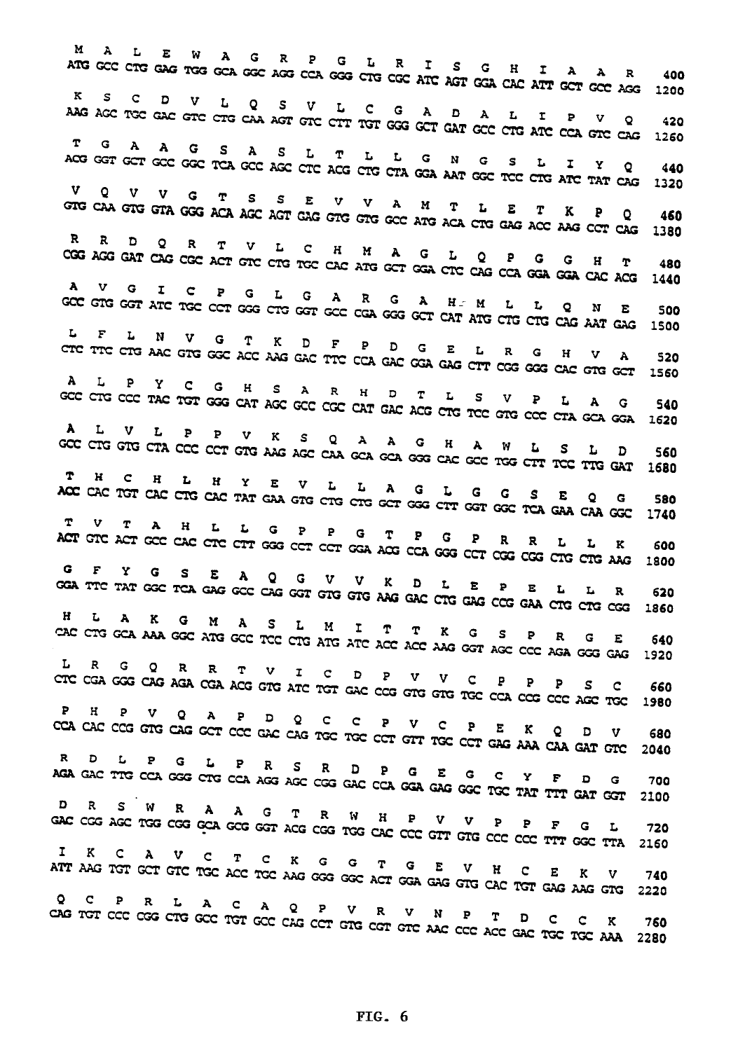

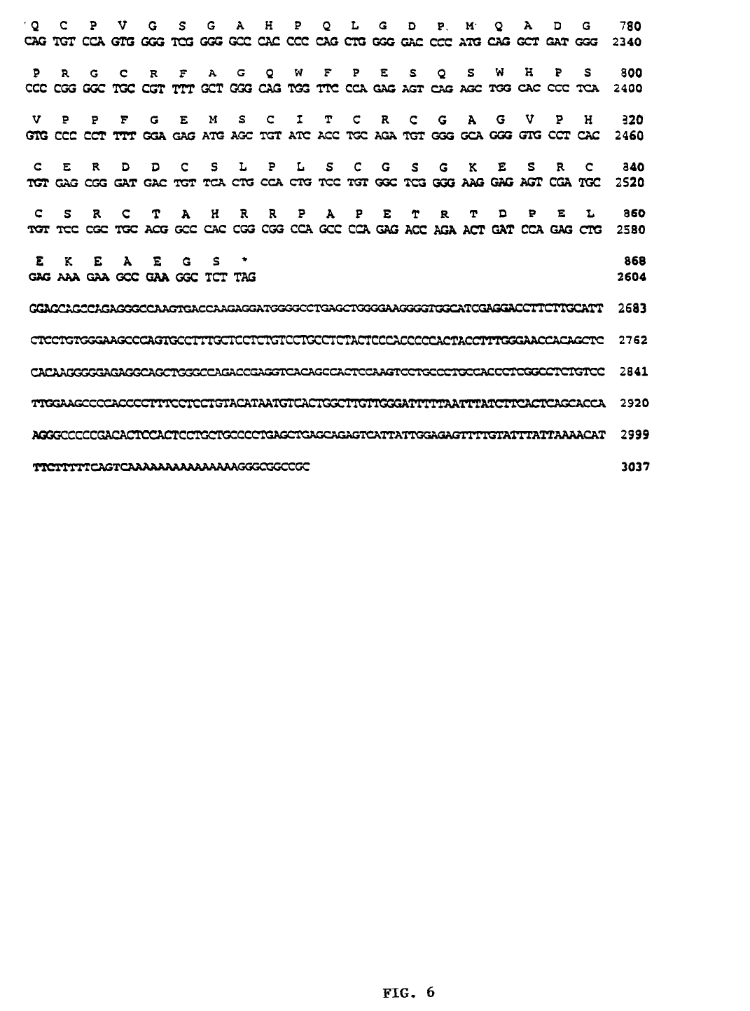

A novel open reading frame was identified during genomic sequencing of a human bacterial artificial chromosome. The open reading frame was located approximately 4 kb upstream of the thrombopoietin gene. A genomic fragment within the open reading frame was used to probe a human brain cDNA library (Clontech; Palo Alto, Calif.). A near full-length cDNA clone, lacking only two nucleotides of the initial Met codon, was identified. The identity of the missing nucleotides was confirmed by comparison to the genomic sequence. The cDNA clone encoded an 867 amino acid protein. The cDNA sequence of huchordin is shown in FIG. 6 (SEQ ID NO: 9). The huchordin encoding portion of this cDNA extends from nucleotide 1 to nucleotide 2601 (SEQ ID NO: 24). The amino acid sequence of huchordin is also shown in FIG. 6 (SEQ ID NO: 10).

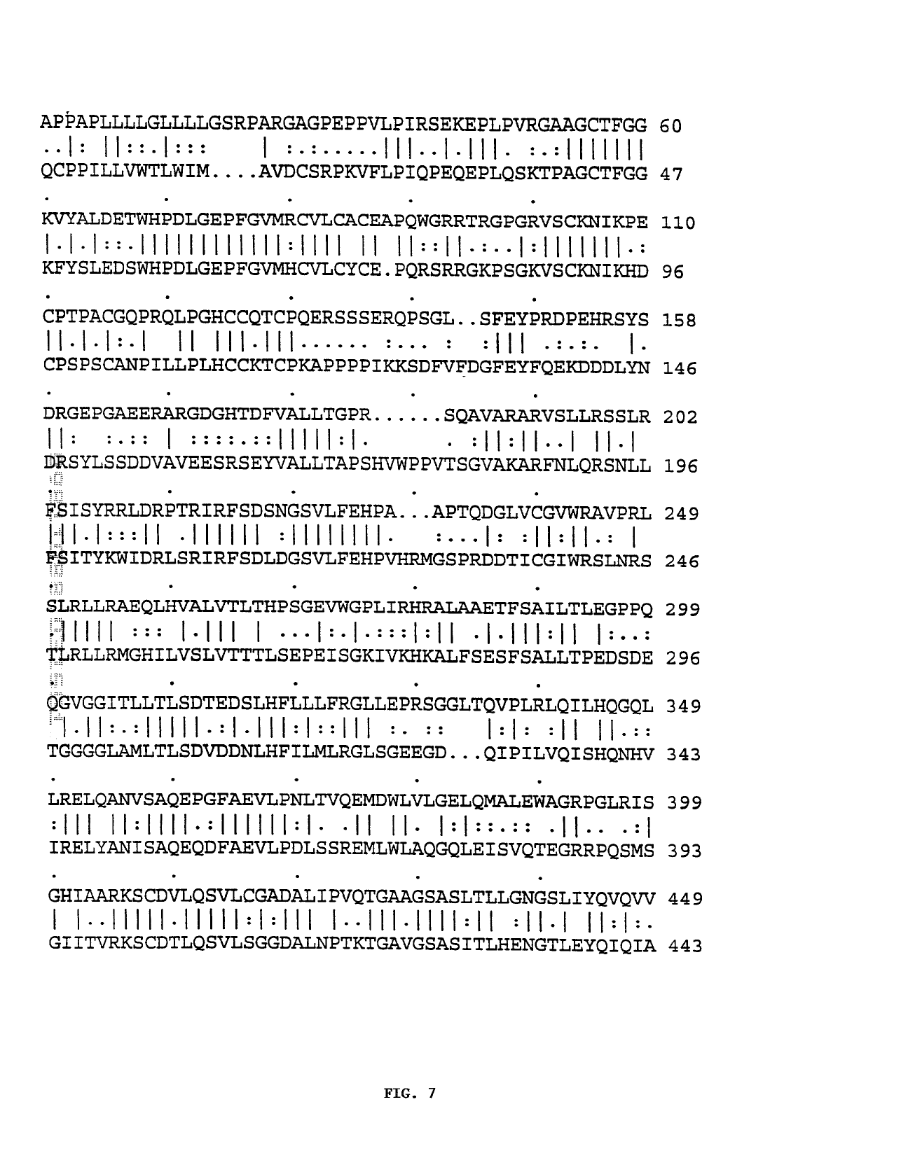

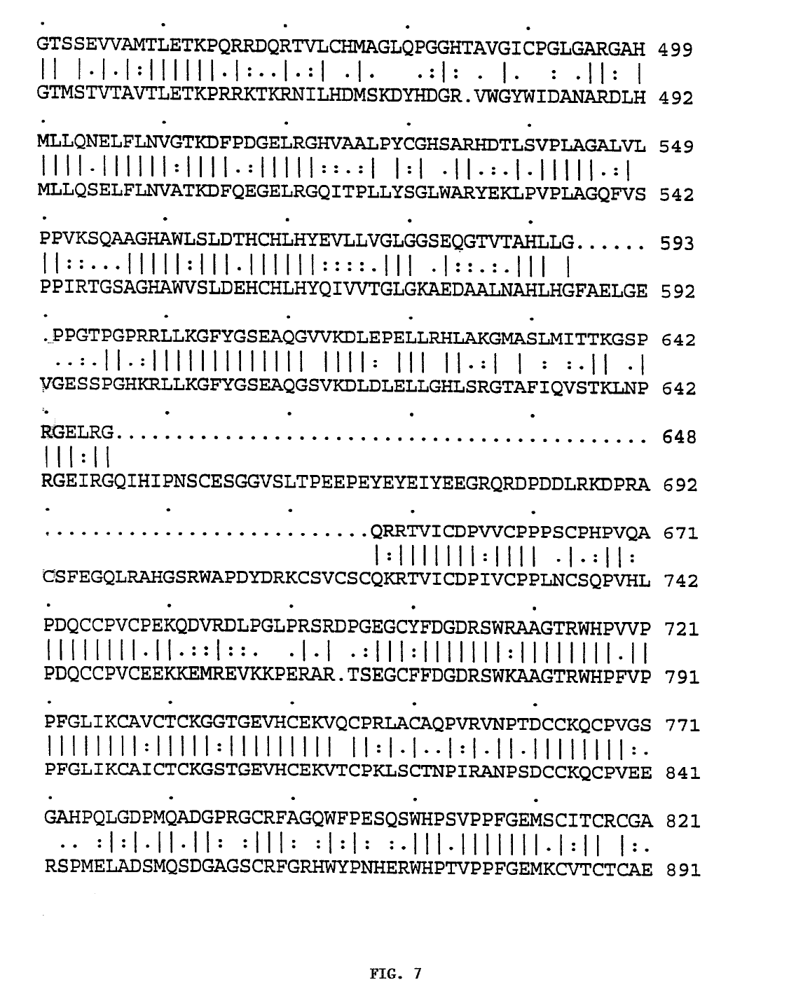

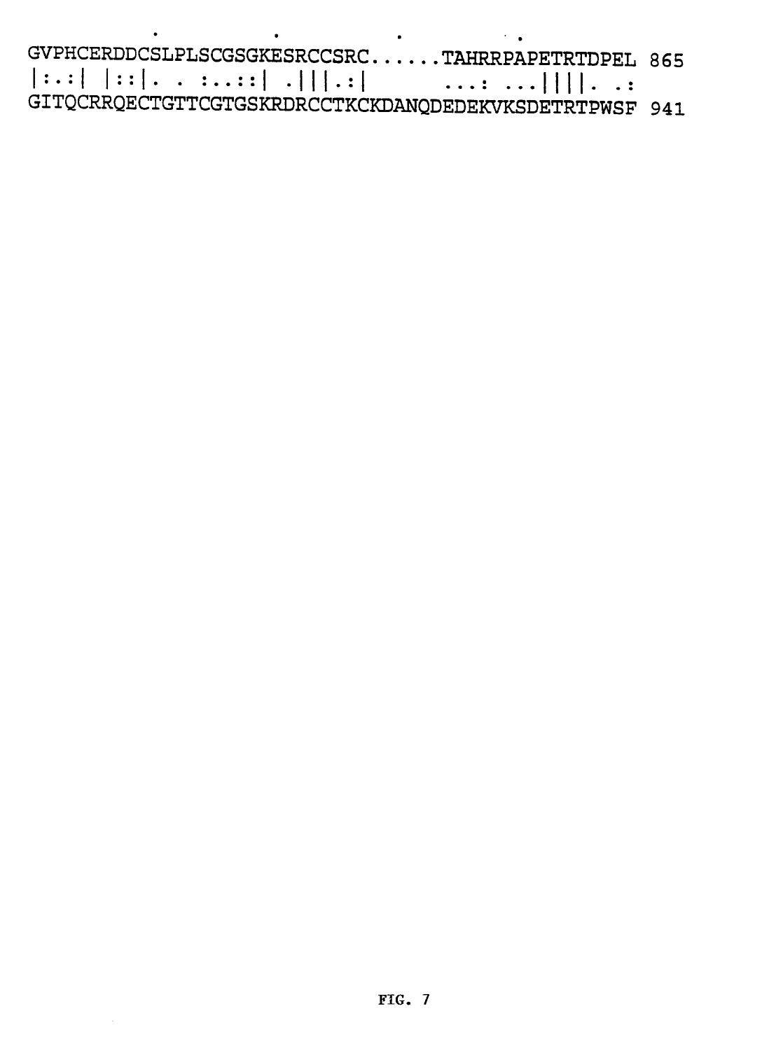

Huchordin is predicted to be a secreted protein having a signal sequence extending from amino acid 1 to amino acid 26. At the amino acid level, huchordin is 53% identical to Xenopus chordin (Sasai et al., Cell 79:779, 1994). FIG. 7 is an alignment of a portion of the amino acid sequence of huchoridin and a portion of the amino acid sequence of Xenopus chordin (SEQ ID NO: 11). Variants of huchordin that are more likely to retain activity do not have alterations at the amino acid positions conserved between huchordin and chordin.

A human Northern blots (Clontech; Palo Alto, Calif.) probed with a full-length huchordin cDNA clone revealed the presence of an approximately 7.5 kb transcript in adult liver and fetal liver, an approximately 2.7 kb transcript in fetal liver, and an approximately 4.4 kb transcript in brain, heart, and pancreas.

As noted above, huchordin has homology to Xenopus chordin, a secreted molecule that functions as a dorsalizing factor in early embryo development. Chordin binds and antagonizes BMP-4, a member of the TGF-beta superfamily.

Huchordin may bind members of the TGF-beta superfamily, e.g., TGF-beta. To the extent that huchordin (or fragments thereof) bind TGF-beta, huchordin can be used to reduce TGF-beta activity, for example, to reduce fibrosis of the kidney, liver, or lung.

The cysteine rich repeats of huchordin are found in thrombospondin-1, thrombospondin-2, and procollagen, protein with anti-angiogenic activity. Thus, huchordin (or fragments thereof which include one or more of the cysteine rich repeats) can be used to inhibit angiogenesis. Such inhibition is useful in limiting tumor growth.

Nucleic Acid Molecules

The nucleic acid molecules of the invention can be cDNA, genomic DNA, synthetic DNA, or RNA, and can be double-stranded or single-stranded (i.e., either a sense or an antisense strand). Fragments of these molecules are also considered within the scope of the invention, and can be produced, for example, by the polymerase chain reaction (PCR) or generated by treatment with one or more restriction endonucleases. A ribonucleic acid (RNA) molecule can be produced by in vitro transcription. Preferably, the nucleic acid molecules encode polypeptides that, regardless of length, are soluble under normal physiological conditions.

The nucleic acid molecules of the invention can contain naturally occurring sequences, or sequences that differ from those that occur naturally, but, due to the degeneracy of the genetic code, encode the same polypeptide (for example, one of the polypeptides of SEQ ID NO: 2, SEQ ID NO: 4, SEQ ID NO: 6, SEQ ID NO: 8, or SEQ ID NO: 10). In addition, these nucleic acid molecules are not limited to sequences that only encode polypeptides, and thus, can include some or all of the non-coding sequences that lie upstream or downstream from a coding sequence.

The nucleic acid molecules of the invention can be synthesized (for example, by phosphoramidite-based synthesis) or obtained from a biological cell, such as the cell of a mammal. Thus, the nucleic acids can be those of a human, mouse, rat, guinea pig, cow, sheep, horse, pig, rabbit, monkey, dog, or cat. Combinations or modifications of the nucleotides within these types of nucleic acids are also encompassed.

In addition, the isolated nucleic acid molecules of the invention encompass fragments that are not found as such in the natural state. Thus, the invention encompasses recombinant molecules, such as those in which a nucleic acid molecule (for example, an isolated nucleic acid molecule encoding a nucleic acid or polypeptide of the invention) is incorporated into a vector (for example, a plasmid or viral vector) or into the genome of a heterologous cell (or the genome of a homologous cell, at a position other than the natural chromosomal location). Recombinant nucleic acid molecules and uses therefor are discussed further below.

In the event the nucleic acid molecules of the invention encode or act as antisense molecules, they can be used for example, to regulate translation of polypeptides of the invention. Techniques associated with detection or regulation of expression of nucleic acids or polypeptides of the invention are well known to skilled artisans and can be used (1) to diagnose and/or treat inflammation or disorders associated with cellular proliferation (e.g., thymotaxin and Tango-67), (2) to diagnose and/or treat disorders associated with apoptotic cell death (e.g., Tango-63), or (3) to diagnose and/or treat disorders associated with aberrant expression of nucleic acids or polypeptides of the invention (e.g., huchordin). These nucleic acid molecules are discussed further below in the context of their clinical utility.

The invention also encompasses nucleic acid molecules that hybridize under stringent conditions to a nucleic acid molecule encoding a polypeptide of the invention. The cDNA sequences described herein (e.g., SEQ ID NO: 1, SEQ ID NO: 3, SEQ ID NO: 5, SEQ ID NO: 7, or SEQ ID NO: 9) can be used to identify these nucleic acids, which include, for example, nucleic acids that encode homologous polypeptides in other species, and splice variants of the genes of the invention in humans or other mammals. Accordingly, the invention features methods of detecting and isolating these nucleic acid molecules. Using these methods, a sample (for example, a nucleic acid library, such as a cDNA or genomic library) is contacted (or “screened”) with a probe specific to a nucleic acid of the invention (for example, a fragment of SEQ ID NO: 1, SEQ ID NO: 3, SEQ ID NO: 5, SEQ ID NO: 7, or SEQ ID NO: 9 that is at least 12 nucleotides long). The probe will selectively hybridize to nucleic acids encoding related polypeptides (or to complementary sequences thereof). Because the polypeptides encoded by nucleic acids of the invention include those related to other C-C chemokines, the term “selectively hybridize” is used to refer to an event in which a probe binds to nucleic acids encoding nucleic acids or polypeptides of the invention (or to complementary sequences thereof) to a detectably greater extent than to nucleic acids encoding other C-C chemokines (or to complementary sequences thereof). The probe, which can contain at least 12 (for example, 15, 25, 50, 100, or 200 nucleotides), can be produced using any of several standard methods (see, for example, Ausubel et al., “Current Protocols in Molecular Biology, Vol. I,” Green Publishing Associates, Inc., and John Wiley & Sons, Inc., NY, 1989). For example, the probe can be generated using PCR amplification methods in which oligonucleotide primers are used to amplify a nucleic acid sequence specific for a nucleic acid or polypeptide of the invention (for example, a nucleic acid encoding the chemokine-like domain) that can be used as a probe to screen a nucleic acid library, as described above, and thereby detect nucleic acid molecules (within the library) that hybridize to the probe.

One single-stranded nucleic acid is said to hybridize to another if a duplex forms between them. This occurs when one nucleic acid contains a sequence that is the reverse and complement of the other (this same arrangement gives rise to the natural interaction between the sense and antisense strands of DNA in the genome and underlies the configuration of the “double helix”). Complete complementarity between the hybridizing regions is not required in order for a duplex to form; it is only necessary that the number of paired bases is sufficient to maintain the duplex under the hybridization conditions used.

Typically, hybridization conditions are of low to moderate stringency. These conditions favor specific interactions between completely complementary sequences, but allow some non-specific interaction between less than perfectly matched sequences to occur as well. After hybridization, the nucleic acids can be “washed” under moderate or high conditions of stringency to dissociate duplexes that are bound together by some non-specific interaction (the nucleic acids that form these duplexes are thus not completely complementary).

As is known in the art, the optimal conditions for washing are determined empirically, often by gradually increasing the stringency. The parameters that can be changed to affect stringency include, primarily, temperature and salt concentration. In general, the lower the salt concentration and the higher the temperature the higher the stringency. Washing can be initiated at a low temperature (for example, room temperature) using a solution containing a salt concentration that is equivalent to or lower than that of the hybridization solution. Subsequent washing can be carried out using progressively warmer solutions having the same salt concentration. As alternatives, the salt concentration can be lowered and the temperature maintained in the washing step, or the salt concentration can be lowered and the temperature increased. Additional parameters can also be altered. For example, use of a destabilizing agent, such as formamide, alters the stringency conditions.

In reactions where nucleic acids are hybridized, the conditions used to achieve a given level of stringency will vary. There is not one set of conditions, for example, that will allow duplexes to form between all nucleic acids that are 85% identical to one another; hybridization also depends on unique features of each nucleic acid. The length of the sequence, the composition of the sequence (for example, the content of purine-like nucleotides versus the content of pyrimidine-like nucleotides) and the type of nucleic acid (for example, DNA or RNA) affect hybridization. An additional consideration is whether one of the nucleic acids is immobilized (for example, on a filter).

An example of a progression from lower to higher stringency conditions is the following, where the salt content is given as the relative abundance of SSC (a salt solution containing sodium chloride and sodium citrate; 2× SSC is 10-fold more concentrated than 0.2× SSC). Nucleic acids are hybridized at 42□ C. in 2× SSC/0.1% SDS (sodium dodecylsulfate; a detergent) and then washed in 0.2× SSC/0.1% SDS at room temperature (for conditions of low stringency); 0.2× SSC/0.1% SDS at 42□ C. (for conditions of moderate stringency); and0.1× SSC at 68□ C. (for conditions of high stringency). Washing can be carried out using only one of the conditions given, or each of the conditions can be used (for example, washing for 10-15 minutes each in the order listed above. Any or all of the washes can be repeated. As mentioned above, optimal conditions will vary and can be determined empirically.

A second set of conditions that are considered “stringent conditions” are those in which hybridization is carried out at 50□ C. in Church buffer (7% SDS, 0.5% NaHPO₄, 1 M EDTA, 1% BSA) and washing is carried out at 50□ C. in 2× SSC.

Once detected, the nucleic acid molecules can be isolated by any of a number of standard techniques (see, for example, Sambrook et al., “Molecular Cloning, A Laboratory Manual,” 2nd Ed. Cold Spring Harbor Laboratory Press, Cold Spring Harbor, N.Y., 1989).

The invention also encompasses: (a) expression vectors that contain any of the foregoing coding sequences related to nucleic acids of the invention and/or their complements (that is, “antisense” sequence); (b) expression vectors that contain any of the foregoing coding sequences related to nucleic acids of the invention operatively associated with a regulatory element (examples of which are given below) that directs the expression of the coding sequences; (c) expression vectors containing, in addition to sequences encoding a polypeptide of the invention, nucleic acid sequences that are unrelated to nucleic acid sequences encoding a nucleic acid or polypeptide of the invention, such as molecules encoding a reporter or marker; and (d) genetically engineered host cells that contain any of the foregoing expression vectors and thereby express the nucleic acid molecules of the invention in the host cell.

Recombinant nucleic acid molecule can contain a sequence encoding a soluble polypeptide of the invention, mature polypeptide of the invention, polypeptide of the invention having a signal sequence, or a domain (e.g., a chemokine-like domain) of a polypeptide of the invention. The full length polypeptides of the invention, a domain of a polypeptide of the invention, or a fragment thereof may be fused to additional polypeptides, as described below. Similarly, the nucleic acid molecules of the invention can encode the mature form of a polypeptide of the invention or a form that encodes a polypeptide that facilitates secretion. In the latter instance, the polypeptide is typically referred to as a proprotein, which can be converted into an active form by removal of the signal sequence, for example, within the host cell. Proproteins can be converted into the active form of the protein by removal of the inactivating sequence.

The regulatory elements referred to above include, but are not limited to, inducible and non-inducible promoters, enhancers, operators and other elements, which are known to those skilled in the art, and which drive or otherwise regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast a-mating factors.

Similarly, the nucleic acid can form part of a hybrid gene encoding additional polypeptide sequences, for example, sequences that function as a marker or reporter. Examples of marker or reporter genes include β-lactamase, chloramphenicol acetyltransferase (CAT), adenosine deaminase (ADA), aminoglycoside phosphotransferase (neo^{r}, G418^{r}), dihydrofolate reductase (DHFR), hygromycin-B-phosphotransferase (HPH), thymidine kinase (TK), lacZ (encoding β-galactosidase), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter. Generally, the hybrid polypeptide will include a first portion and a second portion; the first portion being a polypeptide of the invention and the second portion being, for example, the reporter described above or an immunoglobulin constant region.

The expression systems that may be used for purposes of the invention include, but are not limited to, microorganisms such as bacteria (for example, E. coli and B. subtilis) transformed with recombinant bacteriophage DNA, plasmid DNA, or cosmid DNA expression vectors containing the nucleic acid molecules of the invention; yeast (for example, Saccharomyces and Pichia) transformed with recombinant yeast expression vectors containing the nucleic acid molecules of the invention (preferably containing the nucleic acid sequence of a nucleic acid of the invention (e.g., SEQ ID NO: 1, SEQ ID NO: 3, SEQ ID NO: 5, SEQ ID NO: 7, or SEQ ID NO: 9)); insect cell systems infected with recombinant virus expression vectors (for example, baculovirus) containing the nucleic acid molecules of the invention; plant cell systems infected with recombinant virus expression vectors (for example, cauliflower mosaic virus (CaMV) and tobacco mosaic virus (TMV)) or transformed with recombinant plasmid expression vectors (for example, Ti plasmid) containing nucleotide sequences of a nucleic acid of the invention; or mammalian cell systems (for example, COS, CHO, BHK, 293, VERO, HeLa, MDCK, WI38, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter).

In bacterial systems, a number of expression vectors may be advantageously selected depending upon the use intended for the gene product being expressed. For example, when a large quantity of such a protein is to be produced, for the generation of pharmaceutical compositions containing polypeptides of the invention or for raising antibodies to those polypeptides, vectors that are capable of directing the expression of high levels of fusion protein products that are readily purified may be desirable. Such vectors include, but are not limited to, the E. coli expression vector pUR278 (Ruther et al., EMBO J. 2:1791, 1983), in which the coding sequence of the insert may be ligated individually into the vector in frame with the lacZ coding region so that a fusion protein is produced; pIN vectors (Inouye and Inouye, Nucleic Acids Res. 13:3101-3109, 1985; Van Heeke and Schuster, J. Biol. Chem. 264:5503-5509, 1989); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa califormica nuclear polyhidrosis virus (AcNPV) can be used as a vector to express foreign genes. The virus grows in Spodoptera frugiperda cells. The coding sequence of the insert may be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (for example, see Smith et al., J. Virol. 46:584, 1983; Smith, U.S. Pat. No. 4,215,051).

In mammalian host cells, a number of viral-based expression systems may be utilized. In cases where an adenovirus is used as an expression vector, the nucleic acid molecule of the invention may be ligated to an adenovirus transcription/translation control complex, for example, the late promoter and tripartite leader sequence. This chimeric gene may then be inserted in the adenovirus genome by in vitro or in vivo recombination. Insertion in a non-essential region of the viral genome (for example, region E1 or E3) will result in a recombinant virus that is viable and capable of expressing a gene product of the invention in infected hosts (for example, see Logan and Shenk, Proc. Natl. Acad. Sci. USA 81:3655-3659, 1984). Specific initiation signals may also be required for efficient translation of inserted nucleic acid molecules. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of the coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:516-544, 1987).

In addition, a host cell strain may be chosen which modulates the expression of the inserted sequences, or modifies and processes the gene product in the specific fashion desired. Such modifications (for example, glycosylation) and processing (for example, cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. The mammalian cell types listed above are among those that could serve as suitable host cells.

For long-term, high-yield production of recombinant proteins, stable expression is preferred. For example, cell lines that stably express the nucleic acid or polypeptide sequences of the invention described above may be engineered. Rather than using expression vectors that contain viral origins of replication, host cells can be transformed with DNA controlled by appropriate expression control elements (for example, promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci that in turn can be cloned and expanded into cell lines. This method can advantageously be used to engineer cell lines that express a nucleic acid or polypeptide of the invention. Such engineered cell lines may be particularly useful in screening and evaluation of compounds that affect the endogenous activity of the gene product.

A number of selection systems can be used. For example, the herpes simplex virus thymidine kinase (Wigler, et al., Cell 11:223, 1977), hypoxanthine-guanine phosphoribosyltransferase (Szybalska and Szybalski, Proc. Natl. Acad. Sci. USA 48:2026, 1962), and adenine phosphoribosyltransferase (Lowy, et al., Cell 22:817, 1980) genes can be employed in tk⁻, hgprt⁻ or aprt⁻ cells, respectively. Also, anti-metabolite resistance can be used as the basis of selection for the following genes: dhfr, which confers resistance to methotrexate (Wigler et al., Proc. Natl. Acad. Sci. USA 77:3567, 1980; O′Hare et al., Proc. Natl. Acad. Sci. USA 78:1527, 1981); gpt, which confers resistance to mycophenolic acid (Mulligan and Berg, Proc. Natl. Acad. Sci. USA 78:2072, 1981); neo, which confers resistance to the aminoglycoside G-418 (Colberre-Garapin et al., J. Mol. Biol. 150:1, 1981); and hygro, which confers resistance to hygromycin (Santerre et al., Gene 30:147, 1984).

Alternatively, any fusion protein may be readily purified by utilizing an antibody specific for the fusion protein being expressed. For example, a system described by Janknecht et al. allows for the ready purification of non-denatured fusion proteins expressed in human cell lines (Proc. Natl. Acad. Sci. USA 88: 8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺□nitriloacetic acid-agarose columns and histidine-tagged proteins are selectively eluted with imidazole-containing buffers.

In the event the nucleic acid molecules of the invention encode or act as antisense molecules, they can be used for example, to regulate transcription of the nucleic acid molecules of the invention. For example, with respect to regulation of Tango-63d or Tango-63e transcription, such techniques can be used to diagnose and/or treat disorders associated with apoptotic cell death. These nucleic acids will be discussed further in that context.

In addition to the nucleotide sequences disclosed herein (see, for example SEQ ID NO: 1, SEQ ID NO: 3, SEQ ID NO: 5, SEQ ID NO: 7, or SEQ ID NO: 9), equivalent forms can be present in other species, and can be identified and isolated by using the nucleotide sequences disclosed herein and standard molecular biological techniques. For example, homologs of nucleic acids of the invention can be isolated from other organisms by performing PCR using two degenerate oligonucleotide primer pools designed on the basis of amino acid sequences that are conserved in nucleic acids of the invention. Alternatively, the method used to identify human nucleic acids or polypeptides of the invention can be used to isolate homologs from other species. The template for the reaction can be cDNA obtained by reverse transcription of mRNA prepared from, for example, human or non-human cell lines or tissues, particularly those known or suspected to express nucleic acids or polypeptides of the invention (see expression data presented above). The PCR product can be subcloned and sequenced to ensure that the amplified nucleic acid sequence represents the sequence of a nucleic acid of the invention. Once identified, nucleic acids or polypeptides of the invention in other species can be used, in turn, to develop animal models for the purpose of drug discovery. Alternatively, nucleic acids or polypeptides can be used in in vitro assays for the purpose of drug discovery.

The invention also encompasses nucleotide sequences that encode mutant nucleic acids or polypeptides of the invention, or fragments thereof, that retain one or more functions of nucleic acids or polypeptides of the invention, as described herein.

The invention encompasses peptide nucleic acids (PNA) and PNA-DNA chimeras having the sequence of a portion of a gene of the invention. DNA oligomers and PNA-DNA chimeric oligmers can be used for antisense inhibition (i.e., inhibition of translation) and anti-gene inhibition (i.e., inhibition of transcription) (Hyrup et al., Bioorganic & Medicinal Chem. 4:5, 1996; Finn et al., Nucl. Acids Res. 24: 33357, 1996). PNA oligomer can also be used in DNA pre-gel hybridization as an alternative to Southern hybridization.

The invention encompasses single-stranded nucleic acid probes which hybridize to a nucleic acid molecule of the invention (e.g., the nucleic acid molecule of SEQ ID NO: 1, SEQ ID NO: 3, SEQ ID NO: 5, SEQ ID NO: 7, or SEQ ID NO: 9). Such probes can be used diagnostic methods to detect mutations in the genes of the invention. For example, probes can be used to create a high-density oligonucleotide probe array that can be used diagnostically to detect mutations and allelic variations in genes of the invention (Cronin et al., Human Mutation 7:244, 1996).

As an alternative to screening a cDNA library, a human total genomic DNA library can be screened using probes based on nucleic acids of the invention. Clones positive for a nucleic acid of the invention can then be sequenced and, further, the intron/exon structure of the gene of the invention can be elucidated. Once genomic sequence is obtained, oligonucleotide primers can be designed based on the sequence for use in the isolation, via, for example, Reverse Transcriptase-coupled PCR, of splice variants of nucleic acids of the invention.

Further, a previously unknown gene sequence can be isolated by performing PCR using two degenerate oligonucleotide primer pools designed on the basis of nucleotide sequences within the cDNAs of the invention defined herein. The template for the reaction can be cDNA obtained by reverse transcription of mRNA prepared from human or non-human cell lines or tissue known or suspected to express a gene allele of the invention. The PCR product can be subcloned and sequenced to insure that the amplified sequences represent the sequences of a nucleic-acid-of-the-invention-like gene nucleic acid sequence.

The PCR fragment can then be used to isolate a full-length cDNA clone by a variety of methods. For example, the amplified fragment can be labeled and used to screen a bacteriophage cDNA library. Alternatively, the labeled fragment can be used to screen a genomic library.

PCR technology also can be used to isolate full-length cDNA sequences. For example, RNA can be isolated, following standard procedures, from an appropriate cellular or tissue source. A reverse transcription reaction can be performed on the RNA using an oligonucleotide primer specific for the most 5′ end of the amplified fragment for the priming of first strand synthesis. The resulting RNA/DNA hybrid can then be “tailed” with guanines using a standard terminal transferase reaction, the hybrid can be digested with RNAase H, and second strand synthesis can then be primed with a poly-C primer. Thus, cDNA sequences upstream of the amplified fragment can easily be isolated. For a review of useful cloning strategies, see e.g., Sambrook et al., supra; and Ausubel et al., supra.

In cases where the gene identified is the normal (wild type) gene, this gene can be used to isolate mutant alleles of the gene. Such an isolation is preferable in processes and disorders which are known or suspected to have a genetic basis.

A cDNA of a mutant gene can be isolated, for example, by using PCR, a technique that is well-known to one skilled in the art. In this case, the first cDNA strand can be synthesized by hybridizing an oligo-dT oligonucleotide to mRNA isolated from tissue known or suspected of being expressed in an individual putatively carrying the mutant allele, and by extending the new strand with reverse transcriptase. The second strand of the cDNA can then be synthesized using an oligonucleotide that hybridizes specifically to the 5′-end of the normal gene. Using these two primers, the product is then amplified via PCR, cloned into a suitable vector, and subjected to DNA sequence analysis by methods well known in the art. By comparing the DNA sequence of the mutant gene to that of the normal gene, the mutation(s) responsible for the loss or alteration of function of the mutant gene product can be ascertained.

Alternatively, a genomic or cDNA library can be constructed and screened using DNA or RNA, respectively, from a tissue known to or suspected of expressing the gene of interest in an individual suspected of or known to carry the mutant allele. The normal gene or any suitable fragment thereof can then be labeled and used as a probe to identify the corresponding mutant allele in the library. The clone containing this gene can then be purified through methods routinely practiced in the art, and subjected to sequence analysis using standard techniques as described herein.

Additionally, an expression library can be constructed using DNA isolated from or cDNA synthesized from a tissue known to or suspected of expressing the gene of interest in an individual suspected of or known to carry the mutant allele. In this manner, gene products made by the putatively mutant tissue can be expressed and screened using standard antibody screening techniques in conjunction with antibodies raised against the normal gene product, as described herein. For screening techniques, see, for example, Harlow, E. and Lane, eds., 1988, “Antibodies: A Laboratory Manual,” Cold Spring Harbor Press, Cold Spring Harbor.

In cases where the mutation results in an expressed gene product with altered function (e.g., as a result of a missense mutation), a polyclonal set of antibodies is likely to cross-react with the mutant gene product. Library clones detected via their reaction with such labeled antibodies can be purified and subjected to sequence analysis as described herein.

Nucleic acid molecules of the invention are useful for diagnosis of disorders associated with aberrant expression of nucleic acids or polypeptides of the invention. Nucleic acid molecules of the invention are also useful in genetic mapping and chromosome identification.

Polypeptides

The polypeptides of the invention described herein are those encoded by any of the nucleic acid molecules described above and include fragments, mutants, truncated forms, and fusion proteins of polypeptides of the invention. These polypeptides can be prepared for a variety of uses, including but not limited to (1) the generation of antibodies, (2) as reagents in diagnostic assays, (3) for the identification of other cellular gene products or compounds that can modulate the inflammatory response and as pharmaceutical reagents useful for the treatment of inflammation and certain disorders (see above) that are associated with cellular proliferation (e.g., thymotaxin), (4) for the identification of other cellular gene products involved in the regulation of apoptosis and as reagents in assays for screening for compounds that can be used in the treatment of disorders associated with apoptotic cell death (e.g., Tango-63d or Tango-63e), (5) for the identification of abnormal activity of polypeptides in the TNF receptor superfamily and as pharmaceutical reagents useful in the treatment of such disorders (e.g., Tango-63d or Tango-63e), (6) for the identification of other cellular gene products or compounds that can modulate the activity or expression of a polypeptide of the invention (e.g., Tango-67), or (7) as pharmaceutical reagents useful for the treatment of disorders associated with aberrant expression or activity of nucleic acids or polypeptides of the invention (e.g., Tango-67).

Preferred polypeptides are substantially pure polypeptides of the invention, including those that correspond to the polypeptide with an intact signal sequence (e.g., extending from amino acids 1-24 or 1-29 of SEQ ID NO: 2), the secreted form of the polypeptide (e.g., extending from amino acids 25-97 or 30-97 of SEQ ID NO: 2) of the polypeptides of the invention. Especially preferred are polypeptides that are soluble under normal physiological conditions.

The invention also encompasses polypeptides that are functionally equivalent to a polypeptide of the invention. These polypeptides are equivalent to polypeptides of the invention in that they are capable of carrying out one or more of the functions of polypeptides of the invention in a biological system. Preferred polypeptides of the invention have 20%, 40%, 50%, 75%, 80%, or even 90% of the activity of the full-length, mature form of the polypeptides of the invention described herein. Such comparisons are generally based on an assay of biological activity in which equal concentrations of the polypeptides are used and compared. The comparison can also be based on the amount of the polypeptide required to reach 50% of the maximal stimulation obtainable.

Functionally equivalent proteins can be those, for example, that contain additional or substituted amino acid residues. Substitutions may be made on the basis of similarity in polarity, charge, solubility, hydrophobicity, hydrophilicity, and/or the amphipathic nature of the residues involved. Amino acids that are typically considered to provide a conservative substitution for one another are specified in the summary of the invention.

Polypeptides that are functionally equivalent to polypeptides of the invention (e.g., SEQ ID NO: 2, SEQ ID NO: 4, SEQ ID NO: 6, SEQ ID NO: 8, or SEQ ID NO: 10) can be made using random mutagenesis techniques well known to those skilled in the art (and the resulting mutant polypeptides of the invention can be tested for activity). It is more likely, however, that such polypeptides will be generated by site-directed mutagenesis (again using techniques well known to those skilled in the art). These polypeptides may have an increased functionality (e.g., a greater ability to inhibit cellular proliferation, or to evoke an inflammatory response (e.g, thymotaxin and Tango-67)) or decreased functionality. Polypeptides of the invention show various functionalities (e.g., use for protecting progenitor cells from the effects of chemotherapy and/or radiation therapy).

To design functionally equivalent polypeptides, it is useful to distinguish between conserved positions and variable positions. This can be done by aligning the sequence of cDNAs of the invention obtained from various organisms. Skilled artisans will recognize that conserved amino acid residues are more likely to be necessary for preservation of function. Thus, it is preferable that conserved residues are not altered.

Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Mutations within the coding sequence of nucleic acids of the invention can be made to generate nucleic acids or polypeptides that are better suited for expression in a selected host cell. For example, N-linked glycosylation sites can be altered or eliminated to achieve, for example, expression of a homogeneous product that is more easily recovered and purified from yeast hosts known to hyperglycosylate N-linked sites. To this end, a variety of amino acid substitutions at one or both of the first or third amino acid positions of any one or more of the glycosylation recognition sequences which occur (in N-X-S or N-X--), and/or an amino acid deletion at the second position of any one or more of such recognition sequences, will prevent glycosylation at the modified tripeptide sequence (see, for example, Miyajima et al., EMBO J. 5:1193, 1986).

The polypeptides of the invention can be expressed fused to another polypeptide, for example, a marker polypeptide or fusion partner. For example, the polypeptide can be fused to a hexa-histidine tag to facilitate purification of bacterially expressed protein or a hemagglutinin tag to facilitate purification of protein expressed in eukaryotic cells. The polypeptides of the invention, or a portion thereof, can also be altered so that it has a longer circulating half-life by fusion to an immunoglobulin Fc domain (Capon et al., Nature 337:525-531, 1989). Similarly, a dimeric form of the polypeptides of the inventiion can be produced, which has increased stability in vivo.

Alternatively, a fusion protein may be readily purified by utilizing an antibody specific for the fusion protein being expressed. For example, a system described by Janknecht et al. allows for the ready purification of non-denatured fusion proteins expressed in human cell lines (Proc. Natl. Acad. Sci. USA 88: 8972-8976, 1991). In this system, the gene of interest is subeloned into a vaccinia recombination plasmid such that the gene's open reading frame is translationally fused to an amino-terminal tag consisting of six histidine residues. Extracts from cells infected with recombinant vaccinia virus are loaded onto Ni²⁺□nitriloacetic acid-agarose columns and histidine-tagged proteins are selectively eluted with imidazole-containing buffers.

The polypeptides of the invention can be chemically synthesized (for example, see Creighton, “Proteins: Structures and Molecular Principles,” W.H. Freeman & Co., NY, 1983), or, perhaps more advantageously, produced by recombinant DNA technology as described herein. For example, large polypeptides, i.e., polypeptides equivalent in size to polypeptides of the invention, can advantageously be produced by recombinant DNA technology including in vitro recombinant DNA techniques, synthetic techniques, and in vivo genetic recombination described herein. For additional guidance, skilled artisans may consult Ausubel et al. (supra), Sambrook et al. (“Molecular Cloning, A Laboratory Manual,” Cold Spring Harbor Press, Cold Spring Harbor, N.Y., 1989), and, particularly for examples of chemical synthesis Gait, M. J. Ed. (“Oligonucleotide Synthesis,” IRL Press, Oxford, 1984), which are incorporated by reference herein in their entirety.

Once the recombinant protein of the invention is expressed, it is isolated. Secreted forms can be isolated from the culture media, while non-secreted forms must be isolated from the host cells. Proteins can be isolated by affinity chromatography. In one example, an anti-protein-of-the-invention antibody (e.g., produced as described herein) is attached to a column and used to isolate the protein of the invention. Lysis and fractionation of protein-of-the-invention-harboring cells prior to affinity chromatography can be performed by standard methods (see, e.g., Ausubel et al., supra). Alternatively, a protein of the invention fusion protein, for example, a protein-of-the-invention-maltose binding protein, a protein-of-the-invention-β-galactosidase, or a protein-of-the-invention-trpE fusion protein, can be constructed and used for isolation of proteins of the invention (see, e.g., Ausubel et al., supra; New England Biolabs, Beverly, Mass.).

Once isolated, the recombinant protein can, if desired, be further purified, e.g., by high performance liquid chromatography using standard techniques (see, e.g., Fisher, Laboratory Techniques In Biochemistry And Molecular Biology, eds., Work and Burdon, Elsevier, 1980).

The invention also features polypeptides that interact with polypeptides of the invention (and the genes that encode them) and thereby alter the function of polypeptides of the invention. Interacting polypeptides can be identified using methods known to those skilled in the art. One suitable method is the “two-hybrid system,” which detects protein interactions in vivo (Chien et al., Proc. Natl. Acad. Sci. USA, 88:9578, 1991). A kit for practicing this method is available from Clontech (Palo Alto, Calif.).

The invention encompasses proteins and polypeptides that have one or more of the functions of naturally-occurring polypeptides of the invention. The functional attributes of polypeptides of the invention may include one or more of the following: the ability to bind TRADD (e.g, Tango-63d or Tango-63e), and the ability to initiate a biochemical reaction that induces apoptosis (e.g., Tango-63d or Tango-63e). Polypeptides having one or more functions of naturally-occurring polypeptides of the invention (i.e., functionally equivalent polypeptides) can include, but are not limited to, polypeptides that contain additions or substitutions of amino acid residues within sequences encoded by the nucleic acid molecules described above (e.g, SEQ ID NO: 1, SEQ ID NO: 3, SEQ ID NO: 5, SEQ ID NO: 7, or SEQ ID NO: 9), or that are encoded by nucleic acid molecules which result in a silent change, and thus produce a functionally equivalent gene product. Amino acid substitutions can be made on the basis of similarity in polarity, charge, solubility, hydrophobicity, hydrophilicity, and/or the amphipathic nature of the residues involved. Amino acids that are typically considered as providing a conservative substitution for one another are specified in the summary of the invention.

Random mutations can be made to DNA of the invention using random mutagenesis techniques well known to those skilled in the art, and the resulting mutant polypeptides tested for activity. Alternatively, site-directed mutations can be engineered using site-directed mutagenesis techniques well known to those skilled in the art. The mutant polypeptides generated can have either an increased ability to function in lieu of polypeptides of the invention, for example, they can have a higher binding affinity for putative extracellular ligands or for intracellular polypeptides with which polypeptides of the invention may interact (e.g., to form a complex that instigates apoptosis).

Also encompassed by the invention are polypeptides encoded by nucleic acid molecules which hybridize under stringent conditions to a nucleic acid molecule having the sequence of SEQ ID NO: 1, SEQ ID NO: 3, SEQ ID NO: 5, SEQ ID NO: 7, or SEQ ID NO: 9; polypeptides encoded by nucleic acid molecules which hybridize under stringent conditions to a nucleic acid molecule having the sequence of SEQ ID NO: 1, SEQ ID NO: 3, SEQ ID NO: 5, SEQ ID NO: 7, or SEQ ID NO: 9; and polypeptides encoded by nucleic acid molecules which hybridize under stringent conditions to a nucleic acid molecule having the sequence of the polypeptide encoding portion of one of the clones designated by ATCC accession numbers 98313, 98368, 98367, or 98481.

Transgenic Animals

Polypeptides of the invention can also be expressed in transgenic animals. These animals represent a model system for the study of disorders that are caused by or exacerbated by overexpression or underexpression of nucleic acids or polypeptides of the invention, and for the development of therapeutic agents that modulate the expression or activity of nucleic acids or polypeptides of the invention.

Transgenic animals can be farm animals (pigs, goats, sheep, cows, horses, rabbits, and the like) rodents (such as rats, guinea pigs, and mice), non-human primates (for example, baboons, monkeys, and chimpanzees), and domestic animals (for example, dogs and cats). Transgenic mice are especially preferred. A transgenic animal is any animal containing cells that bear genetic information received, directly or indirectly, by deliberate genetic manipulation at the subcellular level, such as DNA received by microinjection or by infection with recombinant virus.

It is preferred that the nucleic acid molecule becomes integrated with the animal's chromosomes, but the use of DNA sequences that replicate extrachromosomally, such as might be engineered into yeast artificial chromosomes (YACs) or human artificial chromosomes (HACs), are also contemplated.

Preferably, the transgenic animals of the present invention are produced by introducing a nucleic acid molecule of the invention into single-celled embryos so that the DNA is stably integrated into the DNA of germ-line cells in the mature animal, and inherited in a Mendelian fashion. These animals typically have the ability to transfer the genetic information to their offspring. If the offspring in fact possess some or all of the genetic information delivered to the parent animal, then they, too, are transgenic animals. However, any technique known in the art can be used to introduce a transgene of the invention into animals to produce the founder lines of transgenic animals. Such techniques include, but are not limited to, pronuclear microinjection (U.S. Pat. No. 4,873,191); retrovirus mediated gene transfer into germ lines (Van der Putten et al., Proc. Natl. Acad. Sci., USA 82:6148, 1985); gene targeting into embryonic stem cells (Thompson et al., Cell 56:313, 1989); electroporation of embryos (Lo, Mol. Cell. Biol. 3:1803, 1983); and sperm-mediated gene transfer (Lavitrano et al., 1989, Cell 57:717-723); etc.

The present invention provides for transgenic animals that carry a transgene of the invention in all their cells, as well as animals that carry the transgene in some, but not all of their cells. That is, the invention provides for mosaic animals. The transgene can be integrated as a single transgene or in concatamers, e.g., head-to-head tandems or head-to-tail tandems. The transgene can also be selectively introduced into and activated in a particular cell type (Lasko et al., Proc. Natl. Acad. Sci. USA 89:6232, 1992). The regulatory sequences required for such a cell-type specific activation will depend upon the particular cell type of interest, and will be apparent to those of skill in the art.

When it is desired that the transgene of the invention be integrated into the chromosomal site of the endogenous gene of the invention, gene targeting is preferred. Briefly, when such a technique is to be used, vectors containing some nucleotide sequences homologous to an endogenous gene of the invention are designed for the purpose of integrating, via homologous recombination with chromosomal sequences, into and disrupting the function of the nucleotide sequence of the endogenous gene. The transgene also can be selectively introduced into a particular cell type, thus inactivating the endogenous gene of the invention in only that cell type (Gu et al., Science 265: 103, 1984). The regulatory sequences required for such a cell-type specific inactivation will depend upon the particular cell type of interest, and will be apparent to those of skill in the art. These techniques are useful for preparing “knock outs” having no functional gene of the invention.

Once transgenic animals have been generated, the expression of the recombinant gene of the invention can be assayed utilizing standard techniques. Initial screening may be accomplished by Southern blot analysis or PCR techniques to determine whether integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and RT-PCR. Samples of tissue that expresses the gene of the invention can also be evaluated immunocytochemically using antibodies specific for the transgene product of the invention.

For a review of techniques that can be used to generate and assess transgenic animals, skilled artisans can consult Gordon (Intl. Rev. Cytol. 115:171-229, 1989), and may obtain additional guidance from, for example: Hogan et al. “Manipulating the Mouse Embryo” (Cold Spring Harbor Press, Cold Spring Harbor, N.Y., 1986; Krimpenfort et al., Bio/Technology 9:86, 1991; Palmiter et al., Cell 41:343, 1985; Kraemer et al., “Genetic Manipulation of the Early Mammalian Embryo,” Cold Spring Harbor Press, Cold Spring Harbor, N.Y., 1985; Hammer et al., Nature 315:680, 1985; Purcel et al., Science, 244:1281, 1986; Wagner et al., U.S. Pat. No. 5,175,385; and Krimpenfort et al., U.S. Pat. No. 5,175,384 (the latter two publications are hereby incorporated by reference).

Antibodies

Polypeptides of the invention (or immunogenic fragments or analogs) can be used to raise antibodies useful in the invention; such polypeptides can be produced by recombinant techniques or synthesized (see, for example, “Solid Phase Peptide Synthesis,” supra; Ausubel et al., supra). Antibodies that specifically recognize one or more epitopes of these proteins, or fragments thereof are also encompassed by the invention. In general, the peptides can be coupled to a carrier protein, such as KLH, as described in Ausubel et al., supra, mixed with an adjuvant, and injected into a host mammal. Antibodies can be purified by peptide antigen affinity chromatography.

In particular, various host animals can be immunized by injection with a protein or polypeptide of the invention. Host animals include rabbits, mice, guinea pigs, and rats. Various adjuvants that can be used to increase the immunological response depend on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin, and dinitrophenol. Potentially useful human adjuvants include BCG (bacille Calmette-Guerin) and Corynebacterium parvum. Polyclonal antibodies are heterogeneous populations of antibody molecules that are contained in the sera of the immunized animals.

Antibodies within the invention therefore include polyclonal antibodies and, in addition, monoclonal antibodies, humanized or chimeric antibodies, single chain antibodies, Fab fragments, F(ab′)₂ fragments, molecules produced using a Fab expression library, anti-idiotypic (anti-Id) antibodies, and epitope-binding fragments of any of the above.

Monoclonal antibodies, which are homogeneous populations of antibodies to a particular antigen, can be prepared using the proteins of the invention described above and standard hybridoma technology (see, for example, Kohler et al., Nature 256:495, 1975; Kohler et al., Eur. J. Immunol. 6:511, 1976; Kohler et al., Eur. J. Immunol. 6:292, 1976; Hammerling et al., “Monoclonal Antibodies and T Cell Hybridomas,” Elsevier, NY, 1981; Ausubel et al., supra).

In particular, monoclonal antibodies can be obtained by any technique that provides for the production of antibody molecules by continuous cell lines in culture such as described in Kohler et al., Nature 256:495, 1975, and U.S. Pat. No. 4,376,110; the human B-cell hybridoma technique (Kosbor et al., Immunology Today 4:72, 1983; Cole et al., Proc. Natl. Acad. Sci. USA 80:2026, 1983), and the EBV-hybridoma technique (Cole et al., “Monoclonal Antibodies and Cancer Therapy,” Alan R. Liss, Inc., pp. 77-96, 1983). Such antibodies can be of any immunoglobulin class including IgG, IgM, IgE, IgA, IgD and any subclass thereof. The hybridoma producing the mAb of this invention may be cultivated in vitro or in vivo. The ability to produce high titers of mAbs in vivo makes this the presently preferred method of production.

Once produced, polyclonal or monoclonal antibodies are tested for specific recognition of a polypeptide of the invention by Western blot or immunoprecipitation analysis by standard methods, e.g., as described in Ausubel et al., supra. Antibodies that specifically recognize and bind to a polypeptide of the invention are useful in the invention. For example, such antibodies can be used in an immunoassay to monitor the level of polypeptide of the invention produced by a mammal (for example, to determine the amount or subcellular location of a polypeptide of the invention).

Preferably, antibodies of the invention are produced using fragments of the protein of the invention that lie outside highly conserved regions and appear likely to be antigenic, by criteria such as high frequency of charged residues. In one specific example, such fragments are generated by standard techniques of PCR, and are then cloned into the pGEX expression vector (Ausubel et al., supra). Fusion proteins are expressed in E. coli and purified using a glutathione agarose affinity matrix as described in Ausubel, et al., supra.

In some cases it may be desirable to minimize the potential problems of low affinity or specificity of antisera. In such circumstances, two or three fusions can be generated for each protein, and each fusion can be injected into at least two rabbits. Antisera can be raised by injections in a series, preferably including at least three booster injections.

Antisera is also checked for its ability to immunoprecipitate recombinant proteins of the invention or control proteins, such as glucocorticoid receptor, CAT, or luciferase.

The antibodies can be used, for example, in the detection of the polypeptide of the invention in a biological sample as part of a diagnostic assay. Antibodies also can be used in a screening assay to measure the effect of a candidate compound on expression or localization of a polypeptide of the invention. Additionally, such antibodies can be used in conjunction with the gene therapy techniques described to, for example, evaluate the normal and/or engineered polypeptide-of-the-invention-expressing cells prior to their introduction into the patient. Such antibodies additionally can be used in a method for inhibiting abnormal activity of polypeptides of the invention. Preferably, the antibodies recognize epitopes of polypeptides of the invention that are unique, i.e., are not present on related molecules (e.g., members of the TNF receptor superfamily (e.g., TNFR-1) or more distantly related proteins). Accordingly, the antibodies are preferably raised against a peptide sequence present in a polypeptide of the invention that is not present in related molecules (e.g., members of the TNF receptor superfamily).

In addition, techniques developed for the production of “chimeric antibodies” (Morrison et al., Proc. Natl. Acad. Sci. USA, 81:6851, 1984; Neuberger et al., Nature, 312:604, 1984; Takeda et al., Nature, 314:452, 1984) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. Nos. 4,946,778 and 4,704,692, Bird, Science 242:423-426, 1988; Huston et al., Proc. Natl. Acad. Sci. USA 85:5879-5883, 1988; and Ward et al., Nature 334:544-546, 1989) can be adapted to produce single chain antibodies against a protein or polypeptide of the invention. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments include but are not limited to F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed (Huse et al., Science, 246:1275, 1989) to allow rapid and easy identification of monoclonal Fab fragments with the desired specificity.

Antibodies to a polypeptide of the invention can, in turn, be used to generate anti-idiotype antibodies that resemble, or “mimic”, a portion of a polypeptide of the invention using techniques well known to those skilled in the art (see, e.g., Greenspan et al., FASEB J. 7:437, 1993; Nissinoff, J. Immunol. 147:2429, 1991). For example, antibodies that bind to a polypeptide of the invention and competitively inhibit the binding of a ligand of a polypeptide of the invention can be used to generate anti-idiotypes that resemble a ligand-binding domain of a polypeptide of the invention and, therefore, bind and neutralize a ligand of a polypeptide of the invention. Such neutralizing anti-idiotypic antibodies or Fab fragments of such anti-idiotypic antibodies can be used in therapeutic or diagnostic regimens (e.g, neutralizing anti-idiotypes or Fab fragments of such anti-idiotypes can be used in diagnostic regimens to detect disorders associated with apoptotic cell death).

Antibodies can be humanized by methods known in the art. For example, monoclonal antibodies with a desired binding specificity can be commercially humanized (Scotgene, Scotland; Oxford Molecular, Palo Alto, Calif.). Fully human antibodies, such as those expressed in transgenic animals are also features of the invention (Green et al., Nature Genetics 7:13-21, 1994; see also U.S. Pat. Nos. 5,545,806 and 5,569,825, both of which are hereby incorporated by reference).

The methods described herein in which anti-polypeptide-of-the-invention antibodies are employed may be performed, for example, by utilizing pre-packaged diagnostic kits comprising at least one specific polypeptide-of-the-invention nucleotide sequence or antibody reagent described herein, which may be conveniently used, for example, in clinical settings, to diagnose patients exhibiting symptoms of the disorders described below.

Antisense Nucleic Acids

Treatment regimes based on an “antisense” approach involve the design of oligonucleotides (either DNA or RNA) that are complementary to mRNA of the invention. These oligonucleotides bind to the complementary mRNA transcripts of the invention and prevent translation. Absolute complementarity, although preferred, is not required. A sequence “complementary” to a portion of an RNA, as referred to herein, means a sequence having sufficient complementarily to be able to hybridize with the RNA, forming a stable duplex; in the case of double-stranded antisense nucleic acids, a single strand of the duplex DNA may be tested, or triplex formation may be assayed. The ability to hybridize will depend on both the degree of complementarily and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

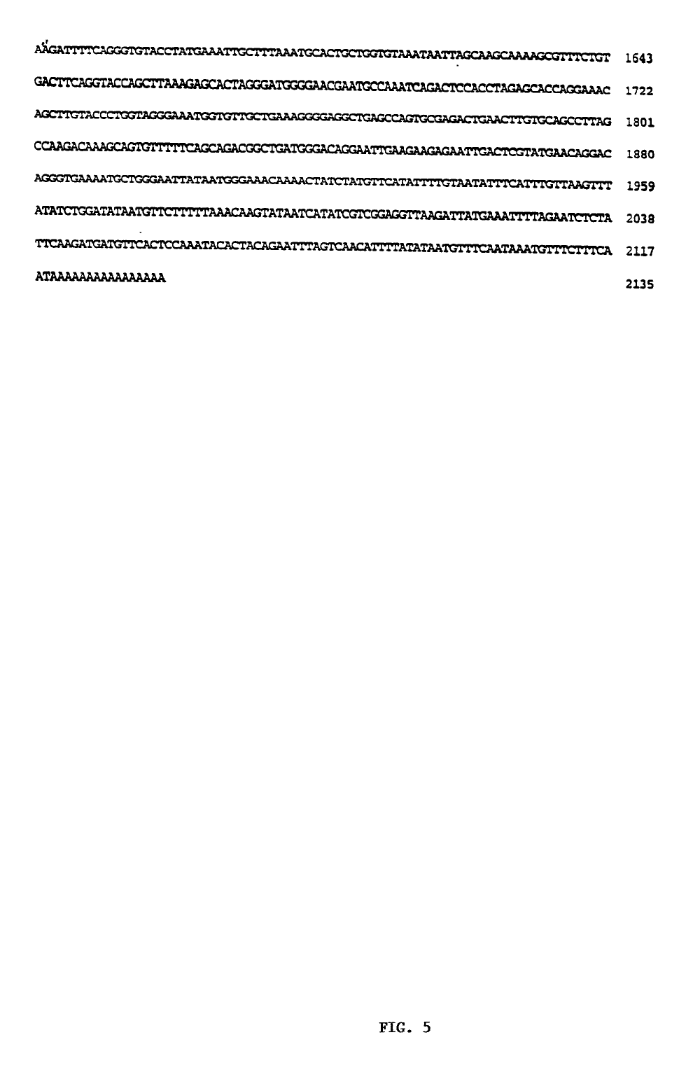

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs recently have been shown to be effective at inhibiting translation of mRNAs as well (Wagner, Nature 372:333, 1984). Thus, oligonucleotides complementary to either the 5′ or 3′ non-translated, non-coding regions of the gene of the invention, e.g., the human gene shown in FIG. 1, FIG. 3, FIG. 4, FIG. 5, or FIG. 6, could be used in an antisense approach to inhibit translation of endogenous thymotaxin, Tango-63d, Tango-63e, Tango-67, or huchordin mRNA. Oligonucleotides complementary to the 5′ untranslated region of the mRNA should include the complement of the AUG start codon.

Antisense oligonucleotides complementary to mRNA coding regions are less efficient inhibitors of translation but could be used in accordance with the invention. Whether designed to hybridize to the 5′, 3′, or coding region of mRNA of the invention, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides, or at least 50 nucleotides.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA, RNA, or PNA, or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (as described, e.g., in Letsinger et al., Proc. Natl. Acad. Sci. USA 86:6553, 1989; Lemaitre et al., Proc. Natl. Acad. Sci. USA 84:648, 1987; PCT Publication No. WO 88/09810) or the blood-brain barrier (see, for example, PCT Publication No. WO 89/10134), or hybridization-triggered cleavage agents (see, for example, Krol et al., BioTechniques 6:958, 1988), or intercalating agents (see, for example, Zon, Pharm. Res. 5:539, 1988). To this end, the oligonucleotide can be conjugated to another molecule, e.g., a peptide, hybridization triggered cross-linking agent, transport agent, or hybridization-triggered cleavage agent.

The antisense oligonucleotide may comprise at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethyl-aminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-theouracil, 2-thiouracil, 4-thiouracil, 5-methyluracil, uracil-5-oxyacetic acid methylester, uracil-5-oxyacetic acid (v), 5-methyl-2-thiouracil, 2-(3-amino-3-N-2-carboxypropl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal, or an analog of any of these backbones.

In yet another embodiment, the antisense oligonucleotide is an α-anomeric oligonucleotide. An a-anomeric oligonucleotide forms specific double-stranded hybrids with complementary RNA in which, contrary to the usual β-units, the strands run parallel to each other (Gautier et al., Nucl. Acids. Res. 15:6625, 1987). The oligonucleotide is a 2′-0-methylribonucleotide (Inoue et al., Nucl. Acids Res. 15:6131, 1987), or a chimeric RNA-DNA analog (Inoue et al., FEBS Lett. 215:327, 1987).

Peptide nucleic acid (PNA) oligonucleotides can be used as antisense molecules (Hyrup et al., Bioorganic & Medicinal Chem. 4:5, 1996).

Antisense oligonucleotides of the invention can be synthesized by standard methods known in the art, e.g., by use of an automated DNA synthesizer (such as are commercially available from Biosearch, Applied Biosystems, etc.). As examples, phosphorothioate oligonucleotides can be synthesized by the method of Stein et al. (Nucl. Acids Res. 16:3209, 1988), and methylphosphonate oligonucleotides can be prepared by use of controlled pore glass polymer supports (Sarin et al., Proc. Natl. Acad. Sci. USA 85:7448, 1988).

While antisense nucleotides complementary to the coding region sequence of a polypeptide of the invention could be used, those complementary to the transcribed untranslated region are most preferred.

The antisense molecules should be delivered to cells that express nucleic acids or polypeptides of the invention in vivo, e.g., cells of the heart, skeletal muscle, thymus, spleen, and small intestine. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense molecule sufficient to suppress translation of endogenous mRNAs. Therefore, a preferred approach uses a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous transcripts of the invention and thereby prevent translation of the mRNA of the invention. For example, a vector can be introduced in vivo such that it is taken up by a cell and directs the transcription of an antisense RNA. Such a vector can remain episomal or become chromosomally integrated, as long as it can be transcribed to produce the desired antisense RNA.

Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include, but are not limited to: the SV40 early promoter region (Bernoist et al., Nature 290:304, 1981); the promoter contained in the 3′ long terminal repeat of Rous sarcoma virus (Yamamoto et al., Cell 22:787-797, 1988); the herpes thymidine kinase promoter (Wagner et al., Proc. Natl. Acad. Sci. USA 78:1441, 1981); or the regulatory sequences of the metallothionein gene (Brinster et al., Nature 296:39, 1988).

As an illustration, examples of suitable antisense molecules directed against thymotaxin mRNA include:

′5 TGCAGTCAGTAGGCGAGCCAT 3′ (SEQ ID NO: 22) and
5′ GTAATCACGGCAGCAGACGCT 3′ (SEQ ID NO: 23).

Ribozymes

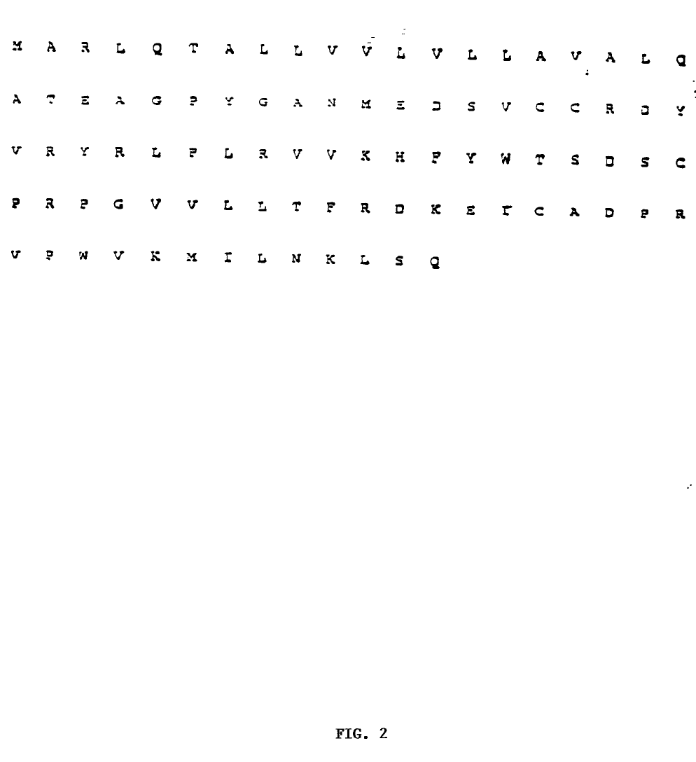

Ribozyme molecules designed to catalytically cleave mRNA transcripts of the invention also can be used to prevent translation of mRNA of the invention and expression of nucleic acids or polypeptides of the invention (see, e.g., PCT Publication WO 90/11364; Saraver et al., Science 247:1222, 1990). While various ribozymes that cleave mRNA at site-specific recognition sequences can be used to destroy mRNAs of the invention, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art (Haseloff et al., Nature 334:585, 1988). There are numerous examples of potential hammerhead ribozyme cleavage sites within the nucleotide sequence of human cDNAs of the invention (e.g., FIG. 2). Preferably, the ribozyme is engineered so that the cleavage recognition site is located near the 5′ end of the mRNA of the invention, i.e., to increase efficiency and minimize the intracellular accumulation of non-functional mRNA transcripts.

As an illustration, examples of potential ribozyme sites in nucleic acids thymotaxin include 5′-UG-3′ sites which correspond to the initiator methionine codon (nucleotides 18-19) and the codons for each of the cysteine residues of the chemokine-like domain (e.g., nucleotides 109-110).

The ribozymes of the present invention also include RNA endoribonucleases (hereinafter “Cech-type ribozymes”), such as the one that occurs naturally in Tetrahymena Thermophila (known as the IVS or L-19 IVS RNA), and which has been extensively described by Cech and his collaborators (Zaug et al., Science 224:574, 1984; Zaug et al., Science, 231:470, 1986; Zug et al., Nature 324:429, 1986; PCT Application No. WO 88/04300; and Been et al., Cell 47:207, 1986). The Cech-type ribozymes have an eight base-pair sequence that hybridizes to a target RNA sequence, whereafter cleavage of the target RNA takes place. The invention encompasses those Cech-type ribozymes that target eight base-pair active site sequences present in nucleic acids or the invention.

As in the antisense approach, the ribozymes can be composed of modified oligonucleotides (e.g., for improved stability, targeting, etc.), and should be delivered to cells that express the nucleic acids or polypeptides of the invention in vivo (e.g., thymotaxin in the heart, skeletal muscle, thymus, spleen, and small intestine). A preferred method of delivery involves using a DNA construct “encoding” the ribozyme under the control of a strong constitutive pol III or pol II promoter, so that transfected cells will produce sufficient quantities of the ribozyme to destroy endogenous messages of nucleic acids or polypeptides of the invention and inhibit translation. Because ribozymes, unlike antisense molecules, are catalytic, a lower intracellular concentration is required for efficiency.

Other Methods for Reducing Expression of Nucleic Acids or Polypeptides of the Invention

A variety of methods can be used to reduce expression of nucleic acids or polypeptides of the invention. For example, the antisense techniques described above can be used to reduce expression of nucleic acids or polypeptides of the invention.

Endogenous expression of genes of the invention can also be reduced by inactivating or “knocking out” the gene of the invention or its promoter using targeted homologous recombination (see, e.g., U.S. Pat. No. 5,464,764). For example, a mutant, non-functional nucleic acid or the invention (or a completely unrelated DNA sequence) flanked by DNA homologous to the endogenous gene of the invention (either the coding regions or regulatory regions of the gene of the invention) can be used, with or without a selectable marker and/or a negative selectable marker, to transfect cells that express a nucleic acid of the invention in vivo. Insertion of the DNA construct, via targeted homologous recombination, results in inactivation of the gene of the invention. Such approaches are particularly suited for use in the agricultural field where modifications to ES (embryonic stem) cells can be used to generate animal offspring with an inactive nucleic acid or polypeptide of the invention. However, this approach can be adapted for use in humans, provided the recombinant DNA constructs are directly administered or targeted to the required site in vivo using appropriate viral vectors.

Alternatively, endogenous expression of a gene of theinvention can be reduced by targeting deoxyribonucleotide sequences complementary to the regulatory region of the gene of the invention (i.e., the promoter and/or enhancers of the gene of the invention) to form triple helical structures that prevent transcription of the gene of the invention in target cells in the body (Helene Anticancer Drug Res. 6:569, 1981; Helene et al., Ann. N.Y. Acad. Sci. 660:27, 1992; and Maher, Bioassays 14:807, 1992) or through the use of small molecules which interfere with the expression or activity of transcription factors which regulate expression of nucleic acids or polypeptides of the invention.

Of course, in some circumstances, including certain phases of many of the above-described conditions, it may be desirable to enhance function of a nucleic acid or polypeptide of the invention, e.g., to recruit immune cells that will resolve the primary infection or mediate an anti-tumor response.

Detecting Proteins Associated with Polypeptides of the Invention

The invention also features polypeptides that interact with polypeptides of the invention. Any method suitable for detecting protein-protein interactions may be employed for identifying transmembrane proteins, intracellular, or extracellular proteins that interact with thymotaxin. Among the traditional methods which may be employed are co-immunoprecipitation, crosslinking and co-purification through gradients or chromatographic columns of cell lysates or proteins obtained from cell lysates and the use of polypeptides of the invention to identify proteins in the lysate that interact with polypeptides of the invention. For these assays, the polypetide of the invention can be a full-length polypeptide of the invention, a soluble extracellular domain of a polypeptide of the invention, or some other suitable polypeptide of the invention. Once isolated, such an interacting protein can be identified and cloned and then used, in conjunction with standard techniques, to identify proteins with which it interacts. For example, at least a portion of the amino acid sequence of a protein that interacts with the polypeptide of the invention can be ascertained using techniques well known to those of skill in the art, such as via the Edman degradation technique. The amino acid sequence obtained may be used as a guide for the generation of oligonucleotide mixtures that can be used to screen for gene sequences encoding the interacting protein. Screening may be accomplished, for example, by standard hybridization or PCR techniques. Techniques for the generation of oligonucleotide mixtures and the screening are well known. (Ausubel, supra; and “PCR Protocols: A Guide to Methods and Applications,” Innis et al., eds. Academic Press, Inc., NY, 1990).

Additionally, methods may be employed which result directly in the identification of genes which encode proteins which interact with polypeptides of the invention. These methods include, for example, screening expression libraries, in a manner similar to the well known technique of antibody probing of λgt 11 libraries, using labeled polypeptide of the invention or a fusion protein of the invention, e.g., a polypeptide or domain of the invention fused to a marker such as an enzyme, fluorescent dye, a luminescent protein, or to an IgFc domain.

There are also methods that are capable of detecting protein interaction. A method that detects protein interactions in vivo is the two-hybrid system (Chien et al., Proc. Natl. Acad. Sci. USA, 88:9578, 1991). A kit for practicing this method is available from Clontech (Palo Alto, Calif.).

Briefly, utilizing such a system, plasmids are constructed that encode two hybrid proteins: one plasmid includes a nucleotide sequence encoding the DNA-binding domain of a transcription activator protein fused to a nucleotide sequence encoding a polypeptide of the invention, a polypeptide of the invention, or a fusion protein of the invention, and the other plasmid includes a nucleotide sequence encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene (e.g., HBS or LacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system, three hybrid system, or related methodology may be used to screen activation domain libraries for proteins that interact with the “bait” gene product. By way of example, and not by way of limitation, a polypeptide of the invention may be used as the bait gene product. Total genomic or cDNA sequences are fused to the DNA encoding an activation domain. This library and a plasmid encoding a hybrid of bait gene product of the invention fused to the DNA-binding domain are cotransformed into a yeast reporter strain, and the resulting transformants are screened for those that express the reporter gene. For example, a bait gene sequence of the invention, such as a nucleic acid of the invention coding for a gene or domain of the invention can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait gene products of the invention are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait gene-of-the-invention-GAL4 fusion plasmid into a yeast strain which contains a lacZ gene driven by a promoter which contains GAL4 activation sequence. A cDNA encoded protein, fused to GAL4 transcriptional activation domain, that interacts with bait gene product of the invention will reconstitute an active GAL4 protein and thereby drive expression of the HIS3 gene. Colonies that express HIS3 can then be purified from these strains, and used to produce and isolate the bait gene-of-the-invention-interacting protein using techniques routinely practiced in the art.

Identification of Receptors of Polypeptides of the Invention

A receptor of a polypeptide of the invention can be identified as follows. First cells or tissues that bind a polypeptide of the invention are identified. An expression library is prepared using mRNA isolated from cells that bind a polypeptide of the invention. The expression library is used to tranfect eukaryotic cells, e.g., CHO cells. Detectably labelled polypeptides of the invention and clones that bind polypeptides of the invention are isolated and purified. The expression plasmid is then isolated from the polypeptide-of-the-invention-binding clones. These expression plasmids will encode putative receptors of polypeptides of the invention.

Cells or tissues bearing a receptor of a polypeptide of the invention can be identified by exposing detectably labelled polypeptide of the invention to various cells lines and tissues. Alternatively a microphysiometer can be used to determine whether a selected cell responds to the presence of a cell receptor ligand (McConnel et al., Science 257:1906, 1992).

Compounds that bind polypeptides of the invention can be identified using any standard binding assay. For example, candidate compounds can be bound to a solid support. The polypeptide of the invention is then exposed to the immobilized compound and binding is measured (European Patent Application 84/03564).

Identification of Compounds that Modulate Expression or Activity of Thymoxin Tango-63, Tango-67, or Huchordin

Isolation of the nucleic acid molecules of the invention also facilitates the identification of compounds that can increase or decrease the expression of these molecules in vivo. To discover such compounds, cells that express nucleic acids or polypeptides of the invention are cultured, exposed to a test compound (or a mixture of test compounds), and the level of expression or activity of nucleic acids or polypeptides of the invention is compared with the level of expression or activity in cells that are otherwise identical but that have not been exposed to the test compound(s). Many standard quantitative assays of gene expression can be utilized in this aspect of the invention. Examples of these assays are provided below.

In order to identify compounds that modulate expression of nucleic acids or polypeptides of the invention (or homologous genes), the candidate compound(s) can be added at varying concentrations to the culture medium of cells that express nucleic acids or polypeptides of the invention, as described above. These compounds can include small molecules, polypeptides, and nucleic acids. The expression of a nucleic acid or polypeptide of the invention is then measured, for example, by Northern blot, PCR analyses or RNAse protection analyses using a nucleic acid molecule of the invention as a probe. The level of expression of the polypeptides of the invention in the presence of the candidate molecule, compared with their level of expression in its absence, will indicate whether or not the candidate molecule alters the expression of nucleic acids or polypeptides of the invention.

Similarly, compounds that modulate the expression of the polypeptides of the invention can be identified by carrying out the assay described above and then performing a Western blot analysis using antibodies that bind polypeptides of the invention.

The test compounds, by altering the expression of nucleic acids or polypeptides of the invention will, in turn, alter the likelihood that the cell in which these molecules are expressed will undergo a cellular process of interest. For example, if the test compound decreases the expression of Tango-63d or Tango-63e, the cell will be less likely to undergo apoptosis. In contrast, if the test compound increases the expression of Tango-63d or Tango-63e, the cell will be more likely to under apoptosis. Thus, compounds identified in this way can be used as agents to control a cellular process of interest (e.g., apoptosis) and, in particular, as therapeutic agents for the treatment of various disorders associated with a cellular process of interest (e.g., apoptosis).

Compounds that alter the activity of nucleic acids or polypeptides of the invention (e.g., by altering the affinity of these polypeptides for putative ligands or other compounds with which they may interact, or alternatively, by changing the fidelity with which they transduce a signal, such as an apoptotic signal) can be identified using an oligomerization or other assay (e.g., an apoptosis assay), such as those described in detail above.

Compounds that can be screened in accordance with the invention include, but are not limited to peptides, antibodies and fragments thereof, and other organic compounds (e.g., 1 o peptidomimetics).

Such compounds can include, but are not limited to, peptides such as, for example, soluble peptides, including but not limited to members of random peptide libraries; (see, e.g., Lam et al., Nature 354:82, 1991; Houghten et al., Nature 354:84, 1991), and combinatorial chemistry-derived molecular library made of D- and/or L-configuration amino acids, phosphopeptides (including, but not limited to, members of random or partially degenerate, directed phosphopeptide libraries; see, e.g., Songyang et al., Cell 72:767, 1993), antibodies (including, but not limited to, polyclonal, monoclonal, humanized, anti-idiotypic, chimeric or single chain antibodies, and FAb, F(ab□)₂ and FAb expression library fragments, and epitope-binding fragments thereof), and small organic or inorganic molecules.

Other compounds that can be screened in accordance with the invention include but are not limited to small organic molecules that affect the expression of a gene of the invention or some other gene involved in a pathway (e.g., signal transduction pathway) involving a gene of the invention (e.g., by interacting with the regulatory region or transcription factors involved in gene expression).

Compounds which Bind Polypeptides of the Invention

Compounds that bind polypeptides of the invention can be identified using any standard binding assay. The principle of the assays used to identify compounds that bind to polypeptides of the invention involves preparing a reaction mixture of polypeptides of the invention and the test compound under conditions and for a time sufficient to allow the two components to interact and bind, thus forming a complex which can be removed and/or detected in the reaction mixture.

The screening assays can be conducted in a variety of ways. For example, one method to conduct such an assay would involve anchoring the protein, polypeptide, peptide, or fusion protein of the invention or the test substance onto a solid phase and detecting polypeptide-of-the-invention/test compound complexes anchored on the solid phase at the end of the reaction. In one embodiment of such a method, a polypeptide of the invention may be anchored onto a solid surface, and the test compound, which is not anchored, may be labeled, either directly or indirectly.

In practice, microtiter plates may conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; for example, using a labeled antibody specific for the previously nonimmobilized component (the antibody, in turn, can be directly labeled or indirectly labeled with a labeled anti-Ig antibody).

Alternatively, a reaction can be conducted in a liquid phase, the reaction products separated from unreacted components, and complexes detected; for example, using an immobilized antibody specific for a protein, polypeptide, peptide, or fusion protein of the invention or the test compound to anchor any complexes formed in solution, and a labeled antibody specific for the other component of the possible complex to detect anchored complexes.

Alternatively, cell-based assays can be used to identify compounds that interact with polypeptides of the invention. To this end, cell lines that express a polypeptide of the invention or cell lines (e.g., COS cells, CHO cells, fibroblasts, etc.) that have been genetically engineered to express a polypeptide of the invention (e.g., by transfection or transduction of DNA of the invention) can be used.

Diagnostic Applications

The polypeptides of the invention and the antibodies specific for these polypeptides are also useful for identifying those compartments of mammalian cells that contain proteins important to the function of nucleic acids or polypeptides of the invention. Antibodies specific for polypeptides of the invention can be produced as described above. The normal subcellular location of the protein is then determined either in situ or using fractionated cells by any standard immunological or immunohistochemical procedure (see, e.g., Ausubel et al., supra; Bancroft and Stevens, Theory and Practice of Histological Techniques, Churchill Livingstone, 1982).

Antibodies specific for a polypeptide of the invention also can be used to detect or monitor diseases related to a nucleic acid or polypeptide of the invention. For example, levels of a protein of the invention in a sample can be assayed by any standard technique using these antibodies. For example, expression of a protein of the invention can be monitored by standard immunological or immunohistochemical procedures (e.g., those described above) using the antibodies described herein. Alternatively, expression of a nucleic acid or polypeptide of the invention can be assayed by standard Northern blot analysis or can be aided by PCR (see, e.g., Ausubel et al., supra; PCR Technology: Principles and Applications for DNA Amplification, ed., H.A. Ehrlich, Stockton Press, NY). If desired or necessary, analysis can be carried out to detect point mutations in the sequence of a nucleic acid or the invention (for example, using well known nucleic acid mismatch detection techniques). All of the above techniques are enabled by the sequences of nucleic acids or the invention described herein.

In addition, the present invention encompasses methods and compositions for the diagnostic evaluation, typing, and prognosis of disorders associated with inappropriate expression or activity of a nucleic acid or polypeptide of the invention. For example, the nucleic acid molecules of the invention can be used as diagnostic hybridization probes to detect, for example, inappropriate expression of a nucleic acid or polypeptide of the invention or mutations in a gene of the invention. Such methods may be used to classify cells by the level of expression of a nucleic acid or polypeptide of the invention.

Thus, the invention features a method for diagnosing a disorder associated with aberrant activity of a nucleic acid or polypeptide of the invention, the method including obtaining a biological sample from a patient and measuring activity of a nucleic acid of polypeptide of the invention in the biological sample, wherein increased or decreased activity of a nucleic acid or polypeptide of the invention in the biological sample compared to a control indicates that the patient suffers from a disorder associated with aberrant activity of a nucleic acid or polypeptide of the invention.

High-density oligonucleotide probe arrays can be used to detect mutations or polymorphism in a gene of the invention. A tiling array (Cronin et al., Human Mutation 7:244, 1996; Kozal et al., Nature Med. 2:753, 1996) can be used to location mutations anywhere in the gene. A mutation array (Cronin et al., Human Mutation 7:244, 1996) can be used to detect the presence of previously identified mutations.

The present invention further provides for diagnostic kits for the practice of such methods.

Therapeutic Applications

Nucleic acid molecules and polypeptides of the invention, and molecules of the invention capable of altering expression, activity, or localization of nucleic acids or polypeptides of the invention can be used to treat a patient suffering from a disorder associated with aberrant expression or activity of a nucleic acid or polypeptide of the invention. Such compounds may be used to treat disorders associated with nucleic acids or polypeptides of the invention (e.g., inhibit fibrosis or angiogenesis).

Therapeutic Compositions

The nucleic acid molecules encoding polypeptides of the invention, the polypeptides themselves, antibodies that specifically bind polypeptides of the invention, and compounds that affect the expression or activity of polypeptides of the invention can be administered to a patient at therapeutically effective doses to treat or ameliorate disorders associated with nucleic acids or polypeptides of the invention. A therapeutically effective dose refers to the dose that is sufficient to result in amelioration of symptoms of disorders associated with nucleic acids or polypeptides of the invention.

Effective Dose

Toxicity and therapeutic efficacy of the polypeptides of the invention and the compounds that modulate their expression or activity can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Polypeptides or other compounds that exhibit large therapeutic indices are preferred. While compounds that exhibit toxic side effects may be used, care should be taken to design a delivery system that targets such compounds to the site of affected tissue in order to minimize potential damage to unaffected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

The compounds may be formulated for parenteral administration by injection, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

The compounds may also be formulated in rectal compositions such as suppositories or retention enemas, for example, containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection.

Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

The therapeutic compositions of the invention can also contain a carrier or excipient, many of which are known to skilled artisans. Excipients which can be used include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. The nucleic acids, polypeptides, antibodies, or modulatory compounds of the invention can be administered by any standard route of administration. For example, administration can be parenteral, intravenous, subcutaneous, intramuscular, intracranial, intraorbital, opthalmic, intraventricular, intracapsular, intraspinal, intracisternal, intraperitoneal, transmucosal, or oral. The modulatory compound can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences.” It is expected that the preferred route of administration will be intravenous.

It is well known in the medical arts that dosages for any one patient depend on many factors, including the general health, sex, weight, body surface area, and age of the patient, as well as the particular compound to be administered, the time and route of administration, and other drugs being administered concurrently.

Dosages for the polypeptides and antibodies of the invention will vary, but a preferred dosage for intravenous administration is approximately 0.01 mg to 100 mg/ml blood volume. Determination of the correct dosage within a given therapeutic regime is well within the abilities of one of ordinary skill in the art of pharmacology. Skilled artisans will be aided in their determination of an adequate dosage by previous studies. For example, Abraham et al. (J. Amer. Med. Assoc. 273:934-941, 1995) administered TNF-α monoclonal antibody (TNF-α-MAb) at doses ranging from 1 to 15 mg/kg. The antibody was well tolerated by all patients, even though they developed human antimurine antibodies; no serum sickness-like reactions, adverse skin reactions, or systemic allergic reactions developed. Similarly, Rankin et al. (Br. J. Rheumatol. 34:334-342, 1995) administered a single intravenous dose of 0.1, 1.0, or 10 mg/kg of an engineered human antibody, CDP571, which neutralizes human TNF-α. Both studies describe in detail how to evaluate patients who have been treated with antibodies.

Methods of Treatment

Thymotaxin, Tango-63d, Tango-63e, Tango-67, and huchordin polypeptides, nucleic acids, and modulators thereof can be used to modulate the function, morphology, proliferation and/or differentiation of cells in the tissues in which they are expressed. Such molecules can be used to treat disorders associated with abnormal or aberrant metabolism or function of cells in the tissues in which they are expressed. Tissues in which thymotaxin, Tango-63d, Tango-63e, Tango-67, or huchordin are expressed include, for example, pancreas, kidney, testis, heart, brain, liver, placenta, lung, skeletal muscle, or small intestine.

As revealed by Northern blot analysis, thymotaxin, Tango-63, Tango-67, and huchordin are expressed in the brain. Consequently, thymotaxin, Tango-63, Tango-67, and huchordin polypeptides, nucleic acids, and modulators thereof can be used to treat disorders of the brain, such as cerebral edema, hydrocephalus, brain herniations, iatrogenic disease (due to, e.g., infection, toxins, or drugs), inflammations (e.g., bacterial and viral meningitis, encephalitis, and cerebral toxoplasmosis), cerebrovascular diseases (e.g., hypoxia, ischemia, and infarction, intracranial hemorrhage and vascular malformations, and hypertensive encephalopathy), and tumors (e.g., neuroglial tumors, neuronal tumors, tumors of pineal cells, meningeal tumors, primary and secondary lymphomas, intracranial tumors, and medulloblastoma), and to treat injury or trauma to the brain.

As revealed by Northern blot analysis, thymotaxin, Tango-63, and Tango-67 are expressed in skeletal muscle. Consequently, thymotaxin, Tango-63, and Tango-67 polypeptides, nucleic acids, and modulators thereof can be used to treat disorders of skeletal muscle, such as muscular dystrophy (e.g., Duchenne Muscular Dystrophy, Becker Muscular Dystrophy, Emery-Dreifuss Muscular Dystrophy, Limb-Girdle Muscular Dystrophy, Facioscapulohumeral Muscular Dystrophy, Myotonic Dystrophy, Oculopharyngeal Muscular Dystrophy, Distal Muscular Dystrophy, and Congenital Muscular Dystrophy), motor neuron diseases (e.g., Amyotrophic Lateral Sclerosis, Infantile Progressive Spinal Muscular Atrophy, Intermediate Spinal Muscular Atrophy, Spinal Bulbar Muscular Atrophy, and Adult Spinal Muscular Atrophy), myopathies (e.g., inflammatory myopathies (e.g., Dermatomyositis and Polymyositis), Myotonia Congenita, Paramyotonia Congenita, Central Core Disease, Nemaline Myopathy, Myotubular Myopathy, and Periodic Paralysis), and metabolic diseases of muscle (e.g., Phosphorylase Deficiency, Acid Maltase Deficiency, Phosphofructokinase Deficiency, Debrancher Enzyme Deficiency, Mitochondrial Myopathy, Carnitine Deficiency, Carnitine Palmityl Transferase Deficiency, Phosphoglycerate Kinase Deficiency, Phosphoglycerate Mutase Deficiency, Lactate Dehydrogenase Deficiency, and Myoadenylate Deaminase Deficiency).

As revealed by Northern blot analysis, thymotaxin, Tango-63, Tango-67, and huchordin are expressed in the heart. Consequently, thymotaxin, Tango-63, Tango-67, and huchordin nucleic acids, proteins, and modulators thereof can be used to treat heart disorders, e.g., ischemic heart disease, atherosclerosis, hypertension, angina pectoris, Hypertrophic Cardiomyopathy, and congenital heart disease.

As revealed by Northern blot analysis, thymotaxin, Tango-63, Tango-67, and huchordin are expressed in the cardiovascular system. Consequently, thymotaxin, Tango-63, Tango-67, and huchordin polypeptides, nucleic acids, and modulators thereof can be used to treat cardiovascular disorders, such as ischemic heart disease (e.g., angina pectoris, myocardial infarction, and chronic ischemic heart disease), hypertensive heart disease, pulmonary heart disease, valvular heart disease (e.g., rheumatic fever and rheumatic heart disease, endocarditis, mitral valve prolapse, and aortic valve stenosis), congenital heart disease (e.g., valvular and vascular obstructive lesions, atrial or ventricular septal defect, and patent ductus arteriosus), or myocardial disease (e.g., myocarditis, congestive cardiomyopathy, and hypertrophic cariomyopathy).

As revealed by Northern blot analysis, thymotaxin, Tango-63, and Tango-67 are expressed in the spleen. Consequently, thymotaxin, Tango-63, and Tango-67 nucleic acids, proteins, and modulators thereof can be used to modulate the proliferation, differentiation, and/or function of cells that form the spleen, e.g., cells of the splenic connective tissue, e.g., splenic smooth muscle cells and/or endothelial cells of the splenic blood vessels. Thymotaxin, Tango-63, and Tango-67 nucleic acids, proteins, and modulators thereof can also be used to modulate the proliferation, differentiation, and/or function of cells that are processed, e.g., regenerated or phagocytized within the spleen, e.g., erythrocytes and/or B and T lymphocytes and macrophages. Thus, thymotaxin, Tango-63, and Tango-67 nucleic acids, proteins, and modulators thereof can be used to treat spleen, e.g., the fetal spleen, associated diseases and disorders. Examples of splenic diseases and disorders include e.g., splenic lymphoma and/or splenomegaly, and/or phagocytotic disorders, e.g., those inhibiting macrophage engulfinent of bacteria and viruses in the bloodstream.

As revealed by Northern blot analysis, thymotaxin and Tango-63 are expressed in leukocytes. Consequently, thymotaxin and Tango-63 polypeptides, nucleic acids, and modulators thereof can be used to treat leukocytic disorders, such as leukopenias (e.g., neutropenia, monocytopenia, lymphopenia, and granulocytopenia), leukocytosis (e.g., granulocytosis, lymphocytosis, eosinophilia, monocytosis, acute and chronic lymphadenitis), malignant lymphomas (e.g., Non-Hodgkin's lymphomas, Hodgkin's lymphomas, leukemias, agnogenic myeloid metaplasia, multiple myeloma, plasmacytoma, Waldenstrom's macroglobulinemia, heavy-chain disease, monoclonal gammopathy, histiocytoses, eosinophilic granuloma, and angioimmunoblastic lymphadenopathy).

As revealed by Northern blot analysis, thymotaxin, Tango-63, and Tango-67 are expressed in leukocytes. Consequently, thymotaxin, Tango-63, and Tango-67 polypeptides, nucleic acids, and modulators thereof can be used to treat pulmonary (lung) disorders, such as atelectasis, cystic fibrosis, rheumatoid lung disease, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, bronchiolitis, Goodpasture's syndrome, idiopathic pulmonary fibrosis, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

As revealed by Northern blot analysis, thymotaxin, Tango-63, Tango-67, and huchordin are expressed in the pancreas. Consequently, thymotaxin, Tango-63, Tango-67, and huchordin polypeptides, nucleic acids, and modulators thereof can be used to treat pancreatic disorders, such as pancreatitis (e.g., acute hemorrhagic pancreatitis and chronic pancreatitis), pancreatic cysts (e.g., congenital cysts, pseudocysts, and benign or malignant neoplastic cysts), pancreatic tumors (e.g., pancreatic carcinoma and adenoma), diabetes mellitus (e.g., insulin- and non-insulin-dependent types, impaired glucose tolerance, and gestational diabetes), or islet cell tumors (e.g., insulinomas, adenomas, Zollinger-Ellison syndrome, glucagonomas, and somatostatinoma).

As revealed by Northern blot analysis, thymotaxin, Tango-63, and Tango-67 are expressed in the small intestine. Consequently, thymotaxin, Tango-63, and Tango-67 polypeptides, nucleic acids, and modulators thereof can be used to treat intestinal disorders, such as ischemic bowel disease, infective enterocolitis, Crohn's disease, benign tumors, malignant tumors (e.g., argentaffinomas, lymphomas, adenocarcinomas, and sarcomas), malabsorption syndromes (e.g., celiac disease, tropical sprue, Whipple's disease, and abetalipoproteinemia), obstructive lesions, hernias, intestinal adhesions, intussusception, or volvulus.

As revealed by Northern blot analysis, thymotaxin, Tango-63, and Tango-67 are expressed in the colon. Consequently, thymotaxin, Tango-63, and Tango-67 polypeptides, nucleic acids, and modulators thereof can be used to treat colonic disorders, such as congenital anomalies (e.g., megacolon and imperforate anus), idiopathic disorders (e.g., diverticular disease and melanosis coli), vascular lesions (e.g., ischemic colistis, hemorrhoids, angiodysplasia), inflammatory diseases (e.g., colitis (e.g., idiopathic ulcerative colitis, pseudomembranous colitis), and lymphopathia venereum), Crohn's disease, and tumors (e.g., hyperplastic polyps, adenomatous polyps, bronchogenic cancer, colonic carcinoma, squamous cell carcinoma, adenoacanthomas, sarcomas, lymphomas, argentaffinomas, carcinoids, and melanocarcinomas).

As revealed by Northern blot analysis, thymotaxin, Tango-63, Tango-67, and huchordin are expressed in the liver. Consequently, thymotaxin, Tango-63, Tango-67, and huchordin polypeptides, nucleic acids, and modulators thereof can be used to treat hepatic (liver) disorders, such as jaundice, hepatic failure, hereditary hyperbiliruinemias (e.g., Gilbert's syndrome, Crigler-Naijar syndromes and Dubin-Johnson and Rotor's syndromes), hepatic circulatory disorders (e.g., hepatic vein thrombosis and portal vein obstruction and thrombosis), hepatitis (e.g., chronic active hepatitis, acute viral hepatitis, and toxic and drug-induced hepatitis), cirrhosis (e.g., alcoholic cirrhosis, biliary cirrhosis, and hemochromatosis), or malignant tumors (e.g., primary carcinoma, hepatoma, hepatoblastoma, liver cysts, and angiosarcoma).

As revealed by Northern blot analysis, thymotaxin, Tango-63, and Tango-67 are expressed in the kidney. Consequently, thymotaxin, Tango-63, and Tango-67 polypeptides, nucleic acids, and modulators thereof can be used to treat renal (kidney) disorders, such as glomerular diseases (e.g., acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, polycystic kidney disease, neoplasia, sickle cell disease, and chronic inflammatory diseases), tubular diseases (e.g., acute tubular necrosis and acute renal failure, polycystic renal diseasemedullary sponge kidney, medullary cystic disease, nephrogenic diabetes, and renal tubular acidosis), tubulointerstitial diseases (e.g., pyelonephritis, drug and toxin induced tubulointerstitial nephritis, hypercalcemic nephropathy, and hypokalemic nephropathy) acute and rapidly progressive renal failure, chronic renal failure, nephrolithiasis, gout, vascular diseases (e.g., hypertension and nephrosclerosis, microangiopathic hemolytic anemia, atheroembolic renal disease, diffuse cortical necrosis, and renal infarcts), or tumors (e.g., renal cell carcinoma and nephroblastoma).

As revealed by Northern blot analysis, thymotaxin, Tango-63, and Tango-67 are expressed in the the reproductive system. Consequently, thymotaxin, Tango-63, and Tango-67 can be used to treat other reproductive disorders, including ovulation disorder, blockage of the fallopian tubes (e.g., due to pelvic inflammatory disease or endometriosis), disorders due to infections (e.g., toxic shock syndrome, chlamydia infection, Herpes infection, human papillomavirus infection), and ovarian disorders (e.g., ovarian cyst, ovarian fibroma, ovarian endometriosis, ovarian teratoma).

As revealed by Northern blot analysis, thymotaxin, Tango-63, and Tango-67 are expressed in the ovaries. Consequently, thymotaxin, Tango-63, and Tango-67 polypeptides, nucleic acids, and modulators thereof can be used to treat ovarian disorders, such as ovarian endometriosis, non-neoplastic cysts (e.g., follicular and luteal cysts and polycystic ovaries) and tumors (e.g., tumors of surface epithelium, germ cell tumors, ovarian fibroma, sex cord-stromal tumors, and ovarian cancers (e.g., metastatic carcinomas, and ovarian teratoma).

As revealed by Northern blot analysis, thymotaxin, Tango-63, and Tango-67 are expressed in the placenta. Consequently, thymotaxin, Tango-63, and Tango-67 polypeptides, nucleic acids, and modulators thereof can be used to treat placental disorders, such as toxemia of pregnancy (e.g., preeclampsia and eclampsia), placentitis, or spontaneous abortion.

As revealed by Northern blot analysis, thymotaxin, Tango-63, and Tango-67 are expressed in the testes. Consequently, thymotaxin, Tango-63, and Tango-67 polypeptides, nucleic acids, and modulators thereof can be used to treat testicular disorders, such as unilateral testicular enlargement (e.g., nontuberculous, granulomatous orchitis); inflammatory diseases resulting in testicular dysfunction (e.g., gonorrhea and mumps); cryptorchidism; sperm cell disorders (e.g., immotile cilia syndrome and germinal cell aplasia); acquired testicular defects (e.g., viral orchitis); and tumors (e.g., germ cell tumors, interstitial cell tumors, androblastoma, testicular lymphoma and adenomatoid tumors).

As revealed by Northern blot analysis, thymotaxin, Tango-63, and Tango-67 are expressed in the prostate. Consequently, thymotaxin, Tango-63, and Tango-67 polypeptides, nucleic acids, and modulators thereof can be used to treat prostate disorders, such as inflammatory diseases (e.g., acute and chronic prostatitis and granulomatous prostatitis), hyperplasia (e.g., benign prostatic hypertrophy or hyperplasia), or tumors (e.g., carcinomas).

Thymotaxin, Tango-63, and Tango-67 are involved in cellular proliferation. Consequently, thymotaxin, Tango-63, and Tango-67 polypeptides, nucleic acids, and modulators thereof can be used to treat proliferative disorders, i.e., neoplasms or tumors (e.g., a carcinoma, a sarcoma, adenoma, or myeloid leukemia).

Disorders associated with abormal thymotaxin, Tango-63, and Tango-67 activity, for which thymotaxin, Tango-63, and Tango-67 agonists can be used to treat, include proliferative disorders (e.g., carcinoma, lymphoma, e.g., follicular lymphoma), and disorders associated with pathogenic infection, e.g., bacterial (e.g., chlamydia) infection, parasitic infection, and viral infection (e.g., HSV infection). Disorders associated with abnormal thymotaxin and Tango-63 activity also include immune disorders (e.g., immunodeficiency disorders (e.g., HIV) and viral disorders (e.g., infection by HSV).

Disorders associated with abnormal thymotaxin and Tango-63 activity, for which thymotaxin and Tango-63 antagonists can be used to treat include immune disorders, e.g., autoimmune disorders (e.g., arthritis, graft rejection (e.g., allograft rejection), T cell disorders (e.g., AIDS)) and inflammatory disorders (e.g., bacterial infection, psoriasis, septicemia, cerebral malaria, inflammatory bowel disease (e.g., ulcerative colitis, Crohn's disease), arthritis (e.g., rheumatoid arthritis, osteoarthritis), and allergic inflammatory disorders (e.g., asthma, psoriasis)). Disorders associated with abnormal Tango-63 activity also include apoptotic disorders (e.g., rheumatoid arthritis, systemic lupus erythematosus, insulin-dependent diabetes mellitus), cytotoxic disorders, septic shock, cachexia, and proliferative disorders (e.g., B cell cancers stimulated by TNF).

Other Tango-63 associated disorders include TNF related disorders (e.g., acute myocarditis, myocardial infarction, congestive heart failure, T cell disorders (e.g., dermatitis, fibrosis)), differentiative and apoptotic disorders, and disorders related to angiogenesis (e.g., tumor formation and/or metastasis, cancer). Modulators of Tango-63 expression and/or activity can be used to treat such disorders.

Deposit of Microorganisms

Microorganisms containing plasmids bearing cDNA encoding thymotaxin was deposited with the American Type Culture Collection (ATCC), Rockville, Md., on Jan. 31, 1997 and assigned the indicated Accession Number 98313. Two plasmids bearing cDNA encoding Tango-63d and Tango-63e respectively, were deposited with the American Type Culture Collection (12301 Parklawn Drive, Rockville, Md. 20852-1776) on Feb. 13, 1997. The plasmid encoding Tango-63d was assigned accession number 98368, and the plasmid encoding Tango-63e was assigned accession number 98367. E. coli strain fth66 harboring a huchordin cDNA clone was deposited with the American Type Culture Collection on Jul. 2, 1997 and given ATCC Accession No. 98481.

Deposit Statement

The subject cultures have been deposited under conditions that assure that access to the cultures will be available during the pendency of the patent application to one determined by the Commissioner of Patents and Trademarks to be entitled thereto under 37 CFR 1.14 and 35 U.S.C. 122. The deposits are available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposits will be stored and made available to the public in accord with the provisions of the Budapest Treaty for the Deposit of Microorganisms, i.e., they will be stored with all the care necessary to keep them viable and uncontaminated for a period of at least five years after the most recent request for the furnishing of a sample of the deposits, and in any case, for a period of at least 30 (thirty) years after the date of deposit or for the enforceable life of any patent which may issue disclosing the cultures plus five years after the last request for a sample from a deposit. The depositor ackowledges the duty to replace the deposits should the depository be unable to furnish a sample when requested, due to the condition of the deposits. All restrictions on the availability to the public of the subject culture deposits will be irrevocably removed upon the granting of a patent disclosing them.

What is claimed is:

1. An isolated nucleic acid molecule selected from the group consisting of:
a) a nucleic acid molecule having a nucleotide sequence which is at least 90% identical to the nucleotide sequence of any of SEQ ID NOs: 1, 3, 5, 7, and 9, or a complement thereof,
b) a nucleic acid molecule comprising at least 15 nucleotide residues and having a nucleotide sequence identical to at least 15 consecutive nucleotide residues of any of SEQ ID NOs: 1, 3, 5, 7, and 9, or a complement thereof;
c) a nucleic acid molecule which encodes a polypeptide comprising the amino acid sequence of any of SEQ ID NOs:2, 4, 6, 8, and 10;
d) a nucleic acid molecule which encodes a fragment of a polypeptide comprising the amino acid sequence of any of SEQ ID NOs:2, 4, 6, 8, and 10, wherein the fragment comprises at least 10 consecutive amino acid residues of any of SEQ ID NOs:2, 4, 6, 8, and 10;
e) a nucleic acid molecule which encodes a fragment of a polypeptide comprising the amino acid sequence of any of SEQ ID NOs:2, 4, 6, 8, and 10, wherein the fragment comprises consecutive amino acid residues corresponding to at least half of the full length of any of SEQ ID NOs:2, 4, 6, 8, and 10; and
f) a nucleic acid molecule which encodes a naturally occurring allelic variant of a polypeptide comprising the amino acid sequence of any of SEQ ID NOs:2, 4, 6, 8, and 10, wherein the nucleic acid molecule hybridizes with a nucleic acid molecule consisting of the nucleotide sequence of any of SEQ ID NOs: 1, 3, 5, 7, and 9, or a complement thereof under stringent conditions.

2. The isolated nucleic acid molecule of claim 1, which is selected from the group consisting of:
a) a nucleic acid having the nucleotide sequence of any of SEQ ID NOs: 1, 3, 5, 7, and 9, or a complement thereof; and
b) a nucleic acid molecule which encodes a polypeptide having the amino acid sequence of any of SEQ ID NOs: 2, 4, 6, 8, and 10, or a complement thereof.

3. The nucleic acid molecule of claim 1, further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of any of SEQ ID NOs: 2, 4, 6, 8, and 10;
b) a naturally occurring allelic variant of a polypeptide comprising the amino acid sequence of any of SEQ ID NOs: 2, 4, 6, 8, and 10, wherein the polypeptide is encoded by a nucleic acid molecule which hybridizes with a nucleic acid molecule consisting of the nucleotide sequence of any of SEQ ID NOs: 1, 3, 5, 7, and 9, or a complement thereof under stringent conditions; and
c) a polypeptide which is encoded by a nucleic acid molecule comprising a nucleotide sequence which is at least 90% identical to a nucleic acid consisting of the nucleotide sequence of any of SEQ ID NOs: 1, 3, 5, 7, and 9, or a complement thereof.

9. The isolated polypeptide of claim 8 having the amino acid sequence of any of SEQ ID NOs: 2, 4, 6, 8, and 10.

10. The polypeptide of claim 8, wherein the amino acid sequence of the polypeptide further comprises heterologous amino acid residues.

11. An antibody which selectively binds with the polypeptide of claim 8.

12. A method for producing a polypeptide selected from the group consisting of:
a) a polypeptide comprising the amino acid sequence of any of SEQ ID NOs: 2, 4, 6, 8, and 10;
b) a polypeptide comprising a fragment of the amino acid sequence of any of SEQ ID NOs: 2, 4, 6, 8, and 10, wherein the fragment comprises at least 10 contiguous amino acids of any of SEQ ID NOs: 2, 4, 6, 8, and 10; and
c) a naturally occurring allelic variant of a polypeptide comprising the amino acid sequence of any of SEQ ID NOs: 2, 4, 6, 8, and 10, or a complement thereof, wherein the polypeptide is encoded by a nucleic acid molecule which hybridizes with a nucleic acid molecule consisting of the nucleotide sequence of any of SEQ ID NOs: 1, 3, 5, 7, and 9, or a complement thereof under stringent conditions;
the method comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds with a polypeptide of claim 8; and
b) determining whether the compound binds with the polypeptide in the sample.

14. The method of claim 13, wherein the compound which binds with the polypeptide is an antibody.

15. A kit comprising a compound which selectively binds with a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes with the nucleic acid molecule; and
b) determining whether the nucleic acid probe or primer binds with a nucleic acid molecule in the sample.

17. The method of claim 16, wherein the sample comprises mRNA molecules and is contacted with a nucleic acid probe.

18. A kit comprising a compound which selectively hybridizes with a nucleic acid molecule of claim 1 and instructions for use.

19. A method for identifying a compound which binds with a polypeptide of claim 8 comprising the steps of:
a) contacting a polypeptide, or a cell expressing a polypeptide of claim 8 with a test compound; and
b) determining whether the polypeptide binds with the test compound.

20. The method of claim 19, wherein the binding of the test compound to the polypeptide is detected by a method selected from the group consisting of:
a) detection of binding by direct detecting of test compound/polypeptide binding;
b) detection of binding using a competition binding assay; and
c) detection of binding using an assay for an activity characteristic of the polypeptide.

21. A method for modulating the activity of a polypeptide of claim 8 comprising contacting a polypeptide or a cell expressing a polypeptide of claim 8 with a compound which binds with the polypeptide in a sufficient concentration to modulate the activity of the polypeptide.

22. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.

23. An antibody substance which selectively binds with the polypeptide of claim 8.

24. A method of making an antibody substance which selectively binds with the polypeptide of claim 8, the method comprising providing the polypeptide to an immunocompetent vertebrate and thereafter harvesting from the vertebrate blood or serum comprising the antibody substance.

25. A method of making an antibody substance which selectively binds with the polypeptide of claim 8, the method comprising contacting the polypeptide with a plurality of particles which individually comprise an antibody substance and a a nucleic acid encoding the antibody substance, segregating a particle which selectively binds with the polypeptide, and expressing the antibody substance from the nucleic acid of the segregated particle.

26. The isolated nucleic acid of claim 1, wherein the isolated nucleic acid comprises a portion having the nucleotide sequence SEQ ID NO: 1.

27. The isolated nucleic acid of claim 1, wherein the isolated nucleic acid comprises a portion having the nucleotide sequence SEQ ID NO: 3.

28. The isolated nucleic acid of claim 1, wherein the isolated nucleic acid comprises a portion having the nucleotide sequence SEQ ID NO: 5.

29. The isolated nucleic acid of claim 1, wherein the isolated nucleic acid comprises a portion having the nucleotide sequence SEQ ID NO: 7.

30. The isolated nucleic acid of claim 1, wherein the isolated nucleic acid comprises a portion having the nucleotide sequence SEQ ID NO: 9.

31. The isolated polypeptide of claim 8, wherein the amino acid sequence of the isolated polypeptide is SEQ ID NO: 2.

32. The isolated polypeptide of claim 8, wherein the amino acid sequence of the isolated polypeptide is SEQ ID NO: 4.

33. The isolated polypeptide of claim 8, wherein the amino acid sequence of the isolated polypeptide is SEQ ID NO: 6.

34. The isolated polypeptide of claim 8, wherein the amino acid sequence of the isolated polypeptide is SEQ ID NO: 8.

35. The isolated polypeptide of claim 8, wherein the amino acid sequence of the isolated polypeptide is SEQ ID NO: 10.


SEQUENCE LISTING (24 sequences)

SEQ ID NO: 1 - DNA, 755 bp, Homo sapiens
  CDS (1)...(279)
atggctcgcctacagactgcactcctggttgtcctcgtcctccttgctgtggcgcttcaagcaactgaggcaggcccctacggcgccaacatggaagacagcgtctgctgccgtgattacgtccgttaccgtctgcccctgcgcgtggtgaaacacttctactggacctcagactcctgcccgaggcctggcgtggtgttgctaaccttcagggataaggagatctgtgccgatcccagagtgccctgggtgaagatgattctcaataagctgagccaatgaagagcctactctgatgaccgtggccttggctcctccaggaaggctcaggagccctacctccctgccattatagctgctccccgccagaagcctgtgccaactctctgcattccctgatctccatccctgtggctgtcacccttggtcacctccgtgctgtcactgccatctcccccctgacccctctaacccatcctctgcctccctccctgcagtcagagggtcctgttcccatcagcgattcccctgcttaaacccttccatgactccccactgccctaagctgaggtcagtctcccaagcctggcatgtggccctctggatctgggttccatttctgtctccagcctgcccacttcccttcatgaatgttgggttctagctccctgttctccaaacccatactacacatcccacttctgggtctttgcctgggatgttgctgacactcagaaagtcccgtcgacgcggcc

SEQ ID NO: 2 - protein, 93 aa, Homo sapiens
MARLQTALLVVLVLLAVALQATEAGPYGANMEDSVCCRDYVRYRLPLRVVKHFYWTSDSCPRPGVVLLTFRDKEICADPRVPWVKMILNKLSQ

SEQ ID NO: 3 - DNA, 4051 bp, Homo sapiens
  CDS (128)...(1447)
  misc_feature (1)...(4051): n = A,T,C or G
gtcgacccacgcgtccggccggagaacccgcaatctttgcgcccacaaaatacaccgacgatgcccgatctactttaagggctgaaacccacgggcctgagagactataagagcgttccctaccgccatggaacaacggggacagaacgccccggccgcttcgggggcccggaaaaggcacggcccaggacccagggaggcgcggggagccaggcctgggctccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtataaagcacagtggggaagccccagctgtggaggagacggtgacctccagcccagggactcctgcctctccctgttctctctcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaattgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaagtgtgattctcttcaggaagtgagaccttccctggtttaccttttttctggaaaaagcccaactggactccagtcagtaggaaagtgccacaattgtcacatgaccggtactggaagaaactctcccatccaacatcacccagtggatggaacatcctgtaacttttcactgcacttggcattatttttataagctgaatgtgataataaggacactatggaaatgtctggatcattccgtttgtgcgtactttgagatttggtttgggatgtcattgttttcacagcacttttttatcctaatgtaaatgctttatttatttatttgggctacattgtaagatccatctacacagtcgttgtccgacttcacttgatactatatgatatgaaccttttttgggtggggggtgcngggcaattccactctgtctcccaggctggagtgcaatggtgcaatcttggctcactatagccttgacctctgaggctcaagcgattctctcacctcagccatccaaatagctgggaccacaggtgtgcaccaccacgcccggctaattttttgtattttgtctaaatataagggctctctatgttgctcagggtggtctcgaattcctggactcaagcagtctgcccacytcagactcccaaagcggtggaattagargcgtgagcccccatgcttggccttacctttctacyttttataattctgtatgttattattttatgaacatgaagaaactttagtaaatgtacttgtttacatagttatgtgaatagattagataaacataaaaggaggagacatacaatgggggaagaagaagaagtcccctgtaagaagttnacgntctggtttccagccttccctcagatgtactttggcttcaatgattggcaacttctacaggggccagtcttttgaactggacaaccttacaagtatatgagtattatttataggtagttgtttacatatgagtcgggaccaaagagaactggatccacgtgaagtcctgtgtgtggctggtccctacctgggcagtctcatttgcacccatagcccccatctatggacaggctgggacagaggcagatgggttagatcacacataacaatagggtctatgtcatatcccaagtgaacttgagccctgtttgggctcaggagatagaagacaaaatctgtctcccacgtctgccatggcatcaagggggaagagtagatggtgcttgagaatggtgtgaaatggttgccatctcaggagtagatggcccggctcacttctggttatctgtcaccctgagcccatgagctgccttttagggtacagattgcctacttgaggaccttggccgctctgtaagcatctgactcatctcagaaatgtcaattcttaaacactgtggcaacaggacctagaatggctgacgcattaaggttttcttcttgtgtcctgttctattattgttttaagacctcagtaaccatttcagcctctttccagcaaacccttctccatagtatttcagtcatggaaggatcatttatgcaggtagtcattccaggagtttttggtcttttctgtctcaaggcattgtgtgttttgttccgggactggtttgggtgggacaaagttagaattgcctgaagatcacacattcagactgttgtgtctgtggagttttaggagtggggggtgacctttctggtctttgcacttccatcctctcccacttccatctggcatcccacgcgttgtcccctgcacttctggaaggcacagggtgctgctgcctcctggtctttgcctttgctgggccttctgtgcaggacgctcagcctcagggctcagaaggtgccagtccggtcccaggtcccttgtcccttccacagaggccttcctagaagatgcatctagagtgtcagccttatcagtgtttaagatttttcttttatttttaatttttttgagacagaatctcactctctcgcccaggctggagtgcaacggtacgatcttggctcagtgcaacctccgcctcctgggttcaagcgattctcgtgcctcagcctccggagtagctgggattgcaggcacccgccaccacgcctggttaatttttgtatttttagtagagacggggtttcaccatgttggtcaggctggtctcgaactcctgacctcaggtgatccaccttggcctccgaaagtgctgggattacaggcgtgagccaccagccaggccaagctattcttttaaagtaagcttcctgacgacatgaaataattgggggttttgttgtttagttacattaggctttgctatatccccaggccaaatagcatgtgacacaggacagccatagtatagtgtgtcactcgtggttggtgtcctttcatgcttctgccctgtcaaaggtccctatttgaaatgtgttataatacaaacaaggaagcacattgtgtacaaaatacttatgtatttatgaatccatgaccaaattaaatatgaaaccttatataaaaaaaaaaaaaaaaaaaaaaaaaaaaaaaaaaaaaaaaaaaaaaaaaaagggsgggcggccgc

SEQ ID NO: 4 - protein, 440 aa, Homo sapiens
MEQRGQNAPAASGARKRHGPGPREARGARPGLRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIKHSGEAPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPAIEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 5 - DNA, 3964 bp, Homo sapiens
  CDS (128)...(1360)
  misc_feature (1)...(3964): n = A,T,C or G
gtcgacccacgcgtccggccggagaacccgcaatctttgcgcccacaaaatacaccgacgatgcccgatctactttaagggctgaaacccacgggcctgagagactataagagcgttccctaccgccatggaacaacggggacagaacgccccggccgcttcgggggcccggaaaaggcacggcccaggacccagggaggcgcggggagccaggcctgggctccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggcatcatcataggagtcacagttgcagccgtagtcttgattgtggctgtgtttgtttgcaagtctttactgtggaagaaagtccttccttacctgaaaggcatctgctcaggtggtggtggggaccctgagcgtgtggacagaagctcacaacgacctggggctgaggacaatgtcctcaatgagatcgtgagtatcttgcagcccacccaggtccctgagcaggaaatggaagtccaggagccagcagagccaacaggtgtcaacatgttgtcccccggggagtcagagcatctgctggaaccggcagaagctgaaaggtctcagaggaggaggctgctggttccagcaaatgaaggtgatcccactgagactctgagacagtgcttcgatgactttgcagacttggtgccctttgactcctgggagccgctcatgaggaagttgggcctcatggacaatgagataaaggtggctaaagctgaggcagcgggccacagggacaccttgtacacgatgctgataaagtgggtcaacaaaaccgggcgagatgcctctgtccacaccctgctggatgccttggagacgctgggagagagacttgccaagcagaagattgaggaccacttgttgagctctggaaagttcatgtatctagaaggtaatgcagactctgccatgtcctaagtgtgattctcttcaggaagtgagaccttccctggtttaccttttttctggaaaaagcccaactggactccagtcagtaggaaagtgccacaattgtcacatgaccggtactggaagaaactctcccatccaacatcacccagtggatggaacatcctgtaacttttcactgcacttggcattatttttataagctgaatgtgataataaggacactatggaaatgtctggatcattccgtttgtgcgtactttgagatttggtttgggatgtcattgttttcacagcacttttttatcctaatgtaaatgctttatttatttatttgggctacattgtaagatccatctacacagtcgttgtccgacttcacttgatactatatgatatgaaccttttttgggtggggggtgcngggcaattccactctgtctcccaggctggagtgcaatggtgcaatcttggctcactatagccttgacctctgaggctcaagcgattctctcacctcagccatccaaatagctgggaccacaggtgtgcaccaccacgcccggctaattttttgtattttgtctaaatataagggctctctatgttgctcagggtggtctcgaattcctggactcaagcagtctgcccacytcagactcccaaagcggtggaattagargcgtgagcccccatgcttggccttacctttctacyttttataattctgtatgttattattttatgaacatgaagaaactttagtaaatgtacttgtttacatagttatgtgaatagattagataaacataaaaggaggagacatacaatgggggaagaagaagaagtcccctgtaagaagttnacgntctggtttccagccttccctcagatgtactttggcttcaatgattggcaacttctacaggggccagtcttttgaactggacaaccttacaagtatatgagtattatttataggtagttgtttacatatgagtcgggaccaaagagaactggatccacgtgaagtcctgtgtgtggctggtccctacctgggcagtctcatttgcacccatagcccccatctatggacaggctgggacagaggcagatgggttagatcacacataacaatagggtctatgtcatatcccaagtgaacttgagccctgtttgggctcaggagatagaagacaaaatctgtctcccacgtctgccatggcatcaagggggaagagtagatggtgcttgagaatggtgtgaaatggttgccatctcaggagtagatggcccggctcacttctggttatctgtcaccctgagcccatgagctgccttttagggtacagattgcctacttgaggaccttggccgctctgtaagcatctgactcatctcagaaatgtcaattcttaaacactgtggcaacaggacctagaatggctgacgcattaaggttttcttcttgtgtcctgttctattattgttttaagacctcagtaaccatttcagcctctttccagcaaacccttctccatagtatttcagtcatggaaggatcatttatgcaggtagtcattccaggagtttttggtcttttctgtctcaaggcattgtgtgttttgttccgggactggtttgggtgggacaaagttagaattgcctgaagatcacacattcagactgttgtgtctgtggagttttaggagtggggggtgacctttctggtctttgcacttccatcctctcccacttccatctggcatcccacgcgttgtcccctgcacttctggaaggcacagggtgctgctgcctcctggtctttgcctttgctgggccttctgtgcaggacgctcagcctcagggctcagaaggtgccagtccggtcccaggtcccttgtcccttccacagaggccttcctagaagatgcatctagagtgtcagccttatcagtgtttaagatttttcttttatttttaatttttttgagacagaatctcactctctcgcccaggctggagtgcaacggtacgatcttggctcagtgcaacctccgcctcctgggttcaagcgattctcgtgcctcagcctccggagtagctgggattgcaggcacccgccaccacgcctggttaatttttgtatttttagtagagacggggtttcaccatgttggtcaggctggtctcgaactcctgacctcaggtgatccaccttggcctccgaaagtgctgggattacaggcgtgagccaccagccaggccaagctattcttttaaagtaagcttcctgacgacatgaaataattgggggttttgttgtttagttacattaggctttgctatatccccaggccaaatagcatgtgacacaggacagccatagtatagtgtgtcactcgtggttggtgtcctttcatgcttctgccctgtcaaaggtccctatttgaaatgtgttataatacaaacaaggaagcacattgtgtacaaaatacttatgtatttatgaatccatgaccaaattaaatatgaaaccttatataaaaaaaaaaaaaaaaaaaaaaaaaaaaaaaaaaaaaaaaaaaaaaaaaaagggsgggcggccgc

SEQ ID NO: 6 - protein, 411 aa, Homo sapiens
MEQRGQNAPAASGARKRHGPGPREARGARPGLRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 7 - DNA, 2135 bp, Homo sapiens
  CDS (182)...(850)
ccacgcgtccgcgcgggcgctgcgctgaggggacggcgggaggcgcggcctggcctcgcactcaaagccgccgcagcgcgccccgggctcggccgacccggcggggatctaggggtgggcgacttcgcgggaccgtggcgcatgtttcctgggagttactgatcatcttctttgaagaaacatgaagttacactatgttgctgtgcttactctagccatcctgatgttcctgacatggcttccagaatcactgagctgtaacaaagcactctgtgctagtgatgtgagcaaatgcctcattcaggagctctgccagtgccggccgggagaaggcaattgctcctgctgtaaggagtgcatgctgtgtcttggggccctttgggacgagtgctgtgactgtgttggtatgtgtaatcctcgaaattatagtgacacacctccaacttcaaagagcacagtggaggagctgcatgaaccgatcccttctctcttccgggcactcacagaaggagatactcagttgaattggaacatcgtttctttccctgttgcagaagaactttcacatcatgagaatctggtttcatttttagaaactgtgaaccagccacaccaccagaatgtgtctgtccccagcaataatgttcacgcgccttattccagtgacaaagaacacatgtgtactgtggtttattttgatgactgcatgtccatacatcagtgtaaaatatcctgtgagtccatgggagcatccaaatatcgctggtttcataatgcctgctgcgagtgcattggtccagaatgtattgactatggtagtaaaactgtcaaatgtatgaactgcatgttttaaagaagacaaatgcaaaccaaagcaacttagtaaaataataggtataaaaagttattctgtaagtctgttggttgtatcttgtatcagaatcccagtaagttaagttgtaaagactttggaataagtttcttttaaaaatatgacatagccagtgatgtgtttaattatataactgttcttactgattttattgccccctagcaataagccctttcctttgaatacatgtacaactttggtcatatgagaagcaggtgcgcagagaattccttgaaagatctgaggtttttaacatgaagtctgatgtggttttcctctagcattccaaaaggtttttgctttgaaagtgttagcagaagcatgttgatgtgaattatgatttcttcatgtgctactgttagcacactgagtttttatagttgcacatcattcctcattgtgccttgttttatccattttataaatagagtagatatttgatataccactctgataactcatataaaaatatcatcataaaaagcttaatttcatcccttttatgttggttttaaaaggtaaatgcttaccatattttataattgagaactcttacatagtagaatccattctataatacatgtgttgacaaagctttagagaaagtttcctattctcttccatttcccctgcccaaagtgctgacataggcagtgatgaagaatctttaccaagattttcagggtgtacctatgaaattgctttaaatgcactgctggtgtaaataattagcaagcaaaagcgtttctgtgacttcaggtaccagcttaaagagcactagggatggggaacgaatgccaaatcagactccacctagagcaccaggaaacagcttgtaccctggtagggaaatggtgttgctgaaaggggaggctgagccagtgcgagactgaacttgtgcagccttagccaagacaaagcagtgtttttcagcagacggctgatgggacaggaattgaagaagagaattgactcgtatgaacaggacagggtgaaaatgctgggaattataatgggaaacaaaactatctatgttcatattttgtaatatttcatttgttaagtttatatctggatataatgttctttttaaacaagtataatcatatcgtcggaggttaagattatgaaattttagaatctctattcaagatgatgttcactccaaatacactacagaatttagtcaacattttatataatgtttcaataaatgtttctttcaataaaaaaaaaaaaaaaa

SEQ ID NO: 8 - protein, 223 aa, Homo sapiens
MKLHYVAVLTLAILMFLTWLPESLSCNKALCASDVSKCLIQELCQCRPGEGNCSCCKECMLCLGALWDECCDCVGMCNPRNYSDTPPTSKSTVEELHEPIPSLFRALTEGDTQLNWNIVSFPVAEELSHHENLVSFLETVNQPHHQNVSVPSNNVHAPYSSDKEHMCTVVYFDDCMSIHQCKISCESMGASKYRWFHNACCECIGPECIDYGSKTVKCMNCMF

SEQ ID NO: 9 - DNA, 3037 bp, Homo sapiens
  CDS (1)...(2601)
atgccgagcctcccggccccgccggccccgctgctgctcctcgggctgctgctgctcggctcccggccggcccgcggcgccggcccagagccccccgtgctgcccatccgttctgagaaggagccgctgcccgttcggggagcggcaggctgcaccttcggcgggaaggtctatgccttggacgagacgtggcacccggacctaggggagccattcggggtgatgcgctgcgtgctgtgcgcctgcgaggcgcctcagtggggtcgccgtaccaggggccctggcagggtcagctgcaagaacatcaaaccagagtgcccaaccccggcctgtgggcagccgcgccagctgccgggacactgctgccagacctgcccccaggagcgcagcagttcggagcggcagccgagcggcctgtccttcgagtatccgcgggacccggagcatcgcagttatagcgaccgcggggagccaggcgctgaggagcgggcccgtggtgacggccacacggacttcgtggcgctgctgacagggccgaggtcgcaggcggtggcacgagcccgagtctcgctgctgcgctctagcctccgcttctctatctcctacaggcggctggaccgccctaccaggatccgcttctcagactccaatggcagtgtcctgtttgagcaccctgcagcccccacccaagatggcctggtctgtggggtgtggcgggcagtgcctcggttgtctctgcggctccttagggcagaacagctgcatgtggcacttgtgacactcactcacccttcaggggaggtctgggggcctctcatccggcaccgggccctggctgcagagaccttcagtgccatcctgactctagaaggccccccacagcagggcgtagggggcatcaccctgctcactctcagtgacacagaggactccttgcattttttgctgctcttccgagggctgctggaacccaggagtgggggactaacccaggttcccttgaggctccagattctacaccaggggcagctactgcgagaacttcaggccaatgtctcagcccaggaaccaggctttgctgaggtgctgcccaacctgacagtccaggagatggactggctggtgctgggggagctgcagatggccctggagtgggcaggcaggccagggctgcgcatcagtggacacattgctgccaggaagagctgcgacgtcctgcaaagtgtcctttgtggggctgatgccctgatcccagtccagacgggtgctgccggctcagccagcctcacgctgctaggaaatggctccctgatctatcaggtgcaagtggtagggacaagcagtgaggtggtggccatgacactggagaccaagcctcagcggagggatcagcgcactgtcctgtgccacatggctggactccagccaggaggacacacggccgtgggtatctgccctgggctgggtgcccgaggggctcatatgctgctgcagaatgagctcttcctgaacgtgggcaccaaggacttcccagacggagagcttcgggggcacgtggctgccctgccctactgtgggcatagcgcccgccatgacacgctgtccgtgcccctagcaggagccctggtgctaccccctgtgaagagccaagcagcagggcacgcctggctttccttggatacccactgtcacctgcactatgaagtgctgctggctgggcttggtggctcagaacaaggcactgtcactgcccacctccttgggcctcctggaacgccagggcctcggcggctgctgaagggattctatggctcagaggcccagggtgtggtgaaggacctggagccggaactgctgcggcacctggcaaaaggcatggcctccctgatgatcaccaccaagggtagccccagaggggagctccgagggcagagacgaacggtgatctgtgacccggtggtgtgcccaccgcccagctgcccacacccggtgcaggctcccgaccagtgctgccctgtttgccctgagaaacaagatgtcagagacttgccagggctgccaaggagccgggacccaggagagggctgctattttgatggtgaccggagctggcgggcagcgggtacgcggtggcaccccgttgtgcccccctttggcttaattaagtgtgctgtctgcacctgcaaggggggcactggagaggtgcactgtgagaaggtgcagtgtccccggctggcctgtgcccagcctgtgcgtgtcaaccccaccgactgctgcaaacagtgtccagtggggtcgggggcccacccccagctgggggaccccatgcaggctgatgggccccggggctgccgttttgctgggcagtggttcccagagagtcagagctggcacccctcagtgcccccttttggagagatgagctgtatcacctgcagatgtggggcaggggtgcctcactgtgagcgggatgactgttcactgccactgtcctgtggctcggggaaggagagtcgatgctgttcccgctgcacggcccaccggcggccagccccagagaccagaactgatccagagctggagaaagaagccgaaggctcttagggagcagccagagggccaagtgaccaagaggatggggcctgagctggggaaggggtggcatcgaggaccttcttgcattctcctgtgggaagcccagtgcctttgctcctctgtcctgcctctactcccacccccactacctttgggaaccacagctccacaagggggagaggcagctgggccagaccgaggtcacagccactccaagtcctgccctgccaccctcggcctctgtccttggaagccccacccctttcctcctgtacataatgtcactggcttgttgggatttttaatttatcttcactcagcaccaagggcccccgacactccactcctgctgcccctgagctgagcagagtcattattggagagttttgtatttattaaaacatttctttttcagtcaaaaaaaaaaaaaaagggcggccgc

SEQ ID NO: 10 - protein, 867 aa, Homo sapiens
MPSLPAPPAPLLLLGLLLLGSRPARGAGPEPPVLPIRSEKEPLPVRGAAGCTFGGKVYALDETWHPDLGEPFGVMRCVLCACEAPQWGRRTRGPGRVSCKNIKPECPTPACGQPRQLPGHCCQTCPQERSSSERQPSGLSFEYPRDPEHRSYSDRGEPGAEERARGDGHTDFVALLTGPRSQAVARARVSLLRSSLRFSISYRRLDRPTRIRFSDSNGSVLFEHPAAPTQDGLVCGVWRAVPRLSLRLLRAEQLHVALVTLTHPSGEVWGPLIRHRALAAETFSAILTLEGPPQQGVGGITLLTLSDTEDSLHFLLLFRGLLEPRSGGLTQVPLRLQILHQGQLLRELQANVSAQEPGFAEVLPNLTVQEMDWLVLGELQMALEWAGRPGLRISGHIAARKSCDVLQSVLCGADALIPVQTGAAGSASLTLLGNGSLIYQVQVVGTSSEVVAMTLETKPQRRDQRTVLCHMAGLQPGGHTAVGICPGLGARGAHMLLQNELFLNVGTKDFPDGELRGHVAALPYCGHSARHDTLSVPLAGALVLPPVKSQAAGHAWLSLDTHCHLHYEVLLAGLGGSEQGTVTAHLLGPPGTPGPRRLLKGFYGSEAQGVVKDLEPELLRHLAKGMASLMITTKGSPRGELRGQRRTVICDPVVCPPPSCPHPVQAPDQCCPVCPEKQDVRDLPGLPRSRDPGEGCYFDGDRSWRAAGTRWHPVVPPFGLIKCAVCTCKGGTGEVHCEKVQCPRLACAQPVRVNPTDCCKQCPVGSGAHPQLGDPMQADGPRGCRFAGQWFPESQSWHPSVPPFGEMSCITCRCGAGVPHCERDDCSLPLSCGSGKESRCCSRCTAHRRPAPETRTDPELEKEAEGS

SEQ ID NO: 11 - protein, 940 aa, Xenopus laevis
QCPPILLVWTLWIMAVDCSRPKVFLPIQPEQEPLQSKTPAGCTFGGKFYSLEDSWHPDLGEPFGVMHCVLCYCEPQRSRRGKPSGKVSCKNIKHDCPSPSCANPILLPLHCCKTCPKAPPPPIKKSDFVFDGFEYFQEKDDDLYNDRSYLSSDDVAVEESRSEYVALLTAPSHVWPPVTSGVAKARFNLQRSNLLFSITYKWIDRLSRIRFSDLDGSVLFEHPVHRMGSPRDDTICGIWRSLNRSTLRLLRMGHILVSLVTTTLSEPEISGKIVKHKALFSESFSALLTPEDSDETGGGGLAMLTLSDVDDNLHFILMLRGLSGEEGDQIPILVQISHQNHVIRELYANISAQEQDFAEVLPDLSSREMLWLAQGQLEISVQTEGRRPQSMSGIITVRKSCDTLQSVLSGGDALNPTKTGAVGSASITLHENGTLEYQIQIAGTMSTVTAVTLETKPRRKTKRNILHDMSKDYHDGRVWGYWIDANARDLHMLLQSELFLNVATKDFQEGELRGQITPLLYSGLWARYEKLPVPLAGQFVSPPIRTGSAGHAWVSLDEHCHLHYQIVVTGLGKAEDAALNAHLHGFAELGEVGESSPGHKRLLKGFYGSEAQGSVKDLDLELLGHLSRGTAFIQVSTKLNPRGEIRGQIHIPNSCESGGVSLTPEEPEYEYEIYEEGRQRDPDDLRKDPRACSFEGQLRAHGSRWAPDYDRKCSVCSCQKRTVICDPIVCPPLNCSQPVHLPDQCCPVCEEKKEMREVKKPERARTSEGCFFDGDRSWKAAGTRWHPFVPPFGLIKCAICTCKGSTGEVHCEKVTCPKLSCTNPIRANPSDCCKQCPVEERSPMELADSMQSDGAGSCRFGRHWYPNHERWHPTVPPFGEMKCVTCTCAEGITQCRRQECTGTTCGTGSKRDRCCTKCKDANQDEDEKVKSDETRTPWSF

SEQ ID NO: 12 - DNA, 29 bp, Artificial Sequence
  oligonucleotide for PCR
cgggatccggcccctacggcgccaacatg

SEQ ID NO: 13 - DNA, 35 bp, Artificial Sequence
  oligonucleotide for PCR
cggaattctcattggctcagcttattgagaatcat

SEQ ID NO: 14 - DNA, 21 bp, Artificial Sequence
  oligonucleotide for PCR
catggcggtagggaacgctct

SEQ ID NO: 15 - DNA, 21 bp, Artificial Sequence
  oligonucleotide for PCR
gttctgtccccgttgttccat

SEQ ID NO: 16 - DNA, 21 bp, Artificial Sequence
  oligonucleotide for PCR
ggcttccccactgtgctttgt

SEQ ID NO: 17 - DNA, 21 bp, Artificial Sequence
  oligonucleotide for PCR
ggaggtcaccgtctcctccac

SEQ ID NO: 18 - DNA, 18 bp, Artificial Sequence
  oligonucleotide for PCR
atggaacaacggggacag

SEQ ID NO: 19 - DNA, 18 bp, Artificial Sequence
  oligonucleotide for PCR
ttcttcgcactgacacac

SEQ ID NO: 20 - DNA, 20 bp, Artificial Sequence
  oligonucleotide for PCR
atgtcattgttttcacagca

SEQ ID NO: 21 - DNA, 18 bp, Artificial Sequence
  oligonucleotide for PCR
gctcaagcgattctctca

SEQ ID NO: 22 - DNA, 21 bp, Artificial Sequence
  oligonucleotide for PCR
tgcagtcagtaggcgagccat

SEQ ID NO: 23 - DNA, 21 bp, Artificial Sequence
  oligonucleotide for PCR
gtaatcacggcagcagacgct

SEQ ID NO: 24 - DNA, 2601 bp, Homo sapiens
atgccgagcctcccggccccgccggccccgctgctgctcctcgggctgctgctgctcggctcccggccggcccgcggcgccggcccagagccccccgtgctgcccatccgttctgagaaggagccgctgcccgttcggggagcggcaggctgcaccttcggcgggaaggtctatgccttggacgagacgtggcacccggacctaggggagccattcggggtgatgcgctgcgtgctgtgcgcctgcgaggcgcctcagtggggtcgccgtaccaggggccctggcagggtcagctgcaagaacatcaaaccagagtgcccaaccccggcctgtgggcagccgcgccagctgccgggacactgctgccagacctgcccccaggagcgcagcagttcggagcggcagccgagcggcctgtccttcgagtatccgcgggacccggagcatcgcagttatagcgaccgcggggagccaggcgctgaggagcgggcccgtggtgacggccacacggacttcgtggcgctgctgacagggccgaggtcgcaggcggtggcacgagcccgagtctcgctgctgcgctctagcctccgcttctctatctcctacaggcggctggaccgccctaccaggatccgcttctcagactccaatggcagtgtcctgtttgagcaccctgcagcccccacccaagatggcctggtctgtggggtgtggcgggcagtgcctcggttgtctctgcggctccttagggcagaacagctgcatgtggcacttgtgacactcactcacccttcaggggaggtctgggggcctctcatccggcaccgggccctggctgcagagaccttcagtgccatcctgactctagaaggccccccacagcagggcgtagggggcatcaccctgctcactctcagtgacacagaggactccttgcattttttgctgctcttccgagggctgctggaacccaggagtgggggactaacccaggttcccttgaggctccagattctacaccaggggcagctactgcgagaacttcaggccaatgtctcagcccaggaaccaggctttgctgaggtgctgcccaacctgacagtccaggagatggactggctggtgctgggggagctgcagatggccctggagtgggcaggcaggccagggctgcgcatcagtggacacattgctgccaggaagagctgcgacgtcctgcaaagtgtcctttgtggggctgatgccctgatcccagtccagacgggtgctgccggctcagccagcctcacgctgctaggaaatggctccctgatctatcaggtgcaagtggtagggacaagcagtgaggtggtggccatgacactggagaccaagcctcagcggagggatcagcgcactgtcctgtgccacatggctggactccagccaggaggacacacggccgtgggtatctgccctgggctgggtgcccgaggggctcatatgctgctgcagaatgagctcttcctgaacgtgggcaccaaggacttcccagacggagagcttcgggggcacgtggctgccctgccctactgtgggcatagcgcccgccatgacacgctgtccgtgcccctagcaggagccctggtgctaccccctgtgaagagccaagcagcagggcacgcctggctttccttggatacccactgtcacctgcactatgaagtgctgctggctgggcttggtggctcagaacaaggcactgtcactgcccacctccttgggcctcctggaacgccagggcctcggcggctgctgaagggattctatggctcagaggcccagggtgtggtgaaggacctggagccggaactgctgcggcacctggcaaaaggcatggcctccctgatgatcaccaccaagggtagccccagaggggagctccgagggcagagacgaacggtgatctgtgacccggtggtgtgcccaccgcccagctgcccacacccggtgcaggctcccgaccagtgctgccctgtttgccctgagaaacaagatgtcagagacttgccagggctgccaaggagccgggacccaggagagggctgctattttgatggtgaccggagctggcgggcagcgggtacgcggtggcaccccgttgtgcccccctttggcttaattaagtgtgctgtctgcacctgcaaggggggcactggagaggtgcactgtgagaaggtgcagtgtccccggctggcctgtgcccagcctgtgcgtgtcaaccccaccgactgctgcaaacagtgtccagtggggtcgggggcccacccccagctgggggaccccatgcaggctgatgggccccggggctgccgttttgctgggcagtggttcccagagagtcagagctggcacccctcagtgcccccttttggagagatgagctgtatcacctgcagatgtggggcaggggtgcctcactgtgagcgggatgactgttcactgccactgtcctgtggctcggggaaggagagtcgatgctgttcccgctgcacggcccaccggcggccagccccagagaccagaactgatccagagctggagaaagaagccgaaggctct